(19) United States
(12) Patent Application Publication
(10) Pub. No.: US 2015/0111223 A1
(43) Pub. Date: Apr. 23, 2015

(54) METHOD FOR EVALUATION OF PRESENCE OF OR RISK OF COLON TUMORS

(71) Applicant: APPLIED PROTEOMICS, INC., SAN DIEGO, CA (US)

(72) Inventors: John Blume, Bellingham, WA (US); Ryan Benz, Huntington Beach, CA (US); Lisa Croner, San Diego, CA (US); Roslyn Dillon, Cardiff, CA (US); Arlo Z. Randall, San Clemente, CA (US); Jeffrey Jones, Glendale, CA (US); Heather Skor, San Diego, CA (US); Tom Stockfisch, Escondido, CA (US); Bruce Wilcox, Harrisonburg, VA (US); Daniel Ruderman, Los Angeles, CA (US)

(21) Appl. No.: 14/526,181

(22) Filed: Oct. 28, 2014

Related U.S. Application Data
(63) Continuation of application No. PCT/US2013/072691, filed on Dec. 2, 2013.
Application No. PCT/US2013/072691 is a continuation of application No. 14/094,594, filed on Dec. 2, 2013.
(60) Provisional application No. 61/772,979, filed on Mar. 5, 2013.
(60) Provisional application No. 61/732,024, filed on Nov. 30, 2012.
(60) Provisional application No. 61/772,979, filed on Mar. 5, 2013.
(60) Provisional application No. 61/732,024, filed on Nov. 30, 2012.

(51) Int. Cl. G01N 33/574

(52) U.S. Cl. CPC G01N 33/57419
USPC 435/7.4; 436/501

(57) ABSTRACT

The disclosed methods are used to predict or assess colon tumor status in a patient. They can be used to determine nature of tumor, recurrence, or patient response to treatments. Some embodiments of the methods include generating a report for clinical management. The methodology provided herein is intended to detect technical variations and to allow for data normalization and enhance signal detection and build predictive proteins profiles of disease status and response.

### CROSS-REFERENCE

This application claims priority under 35 U.S.C. §119(e) to U.S. Provisional Application Nos. 61/732,024, filed on Nov. 30, 2012, and 61/772,979 filed on Mar. 5, 2013, all of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Nov. 27, 2013, is named 36765-703.201_SL.txt and is 783,936 bytes in size.

### BACKGROUND OF THE DISCLOSURE

As is known in the field, the information content of the genome is carried as DNA. The first step of gene expression is the transcription of DNA into mRNA. The second step in gene expression is the synthesis of polypeptide from mRNA, such that every three nucleotides of mRNA encodes for one amino acid residue that will make up the polypeptide. After translation, polypeptides are often post-translationally modified by the addition of different chemical groups such as carbohydrate, lipid and phosphate groups, as well as through the proteolytic cleavage of specific peptide bonds. These chemical modifications allow the polypeptide to assume a unique three-dimensional conformation giving rise to the mature protein. While these post-translational modifications are not directly coded for from the mRNA template, they are pivotal attributes of the protein that act to modulate its function by changing overall conformation and available interaction sites. Moreover, protein levels within a cell can reflect whether an individual is in a healthy or disease state. Consequently, proteins are a very valuable source of biomarkers of disease status, early onset of disease, and risk of disease.

Both mRNA and protein are continually being synthesized and degraded by separate pathways. In addition, there are multiple levels of regulation on the synthesis and degradation pathways. Given this, it is not surprising that there is no simple correlation between the abundance of mRNA species and the actual amounts of proteins for which they code (Anderson and Seilhamer, Electrophoresis 18: 533-537; Gygi et al., Mol. Cell. Biol. 19: 1720-1730, 1999). Thus, while mRNA levels are often extrapolated to indicate the levels of expressed proteins, final levels of protein are not necessarily obtainable by measuring mRNA levels (Patton, J. Chromatogr. 722: 203-223, 1999); Patton et al., J. Biol. Chem. 270: 21404-21410 (1995).

Thus, methods of determining the protein profile of biological samples are needed.

### SUMMARY OF THE DISCLOSURE

Methods are disclosed for detecting the presence of an adenoma, cancer, or polyp of the colon in a subject with a sensitivity of greater than 70% or a selectivity of greater than 70%. In various embodiments, said methods comprise the steps of: (a) obtaining a blood sample from a subject; (b) cleaving proteins in said blood sample to provide a sample comprising peptides; (c) analyzing said sample for the presence of at least ten peptides; (d) comparing the results of analyzing said sample with control reference values to determine a positive or negative score for the presence of an adenoma or polyp of the colon with a sensitivity of greater than 70% or a selectivity of greater than 70%. Also disclosed are methods of treating an adenoma, cancer, or polyp of the colon in a subject comprising (a) performing the method of detecting as described herein to yield a subject with a positive score for the presence of an adenoma, cancer, or polyp; and (b) performing a procedure for the removal of adenoma or polyp tissue in said subject.

Additionally, methods are disclosed for detecting the presence or absence of an adenoma or polyp of the colon in a subject, wherein said subject has no symptoms or family history of adenoma or polyps of the colon, said method comprising the steps of: (a) obtaining a biological sample from said subject; (b) performing an analysis of the biological sample for the presence and amount of one or more proteins and/or peptides; (c) comparing the presence and amount of one or more proteins and/or peptides from said biological sample to a control reference value; and (d) correlating the presence and amount of one or more proteins and/or peptides with the subject's adenoma, cancer, or polyp status.

Additionally, methods are disclosed for detecting the presence or absence of an adenoma, cancer, or polyp of the colon in a subject in whom a colonoscopy yielded a negative result comprising the steps of: (a) obtaining a biological sample from a subject with a negative diagnosis of adenoma, cancer, or polyps based on colonoscopy; (b) performing an analysis of the biological sample for the presence and amount of one or more proteins and/or peptides; (c) comparing the presence and amount of one or more proteins and/or peptides from said biological sample to a control reference value; and (d) correlating the presence and amount of one or more proteins and/or peptides with the subject's adenoma, cancer, or polyp status.

Methods are disclosed for detecting recurrence or absence of an adenoma, cancer, or polyp of the colon in a subject previously treated for adenoma, cancer, or polyps of the colon comprising the steps of: (a) obtaining a biological sample from a subject previously treated for adenoma, cancer, or polyps of the colon; (b) performing an analysis of the biological sample for the presence and amount of one or more proteins and/or peptides; (c) comparing the presence and amount of one or more proteins and/or peptides from said biological sample to a control reference value; and (d) correlating the presence and amount of one or more proteins and/or peptides with the subject's adenoma, cancer, or polyp status.

In addition, methods are disclosed for protein and/or peptide detection for diagnostic application comprising the steps of: (a) obtaining a biological sample from a subject; (b) performing an analysis of the biological sample for the presence and amount of one or more proteins and/or peptides; (c) comparing the presence and amount of one or more proteins and/or peptides from said biological sample to a control reference value; and (d) correlating the presence and amount of one or more proteins and/or peptides with a diagnosis for said subject; wherein said analysis detects the presence and amount of one or more proteins, peptides, or classifiers as disclosed herein.

Additional, a kit is disclosed for performing a method as described herein, where the kit contains: (a) a container for collecting a sample from a subject; (b) means for detecting one or more proteins or peptides, or means for transferring said container to a test facility; and (c) written instructions.

Lastly, the present disclosure provide for a method for the diagnosis, prediction, prognosis and/or monitoring a colon disease. Methods are also disclosed for the diagnosis, prediction, prognosis and/or monitoring a colon disease or colorectal cancer in a subject comprising: measuring at least one biomarker selected from the group ACTB, ACTH, ANGT, SAHH, ALDR, AKT1, ALBU, AL1A1, AL1B1, ALDOA, AMY2B, ANXA1, ANXA3, ANXA4, ANXA5, APC, APOA1, APOC1, APOH, GDIR1, ATPB, BANK1, MIC1, CA195, CO3, CO9, CAH1, CAH2, CALR, CAPG, CD24, CD63, CDD, CEAM3, CEAM5, CEAM6, CGHB, CH3L1, KCRB, CLC4D, CLUS, CNN1, COR1C, CRP, CSF1, CTNB1, CATD, CATS, CATZ, CUL1, SYDC, DEFT, DEF3, DESM, DPP4, DPYL2, DYHC1, ECH1, EF2, IF4A3, ENOA, EZRI, NIBL2, SEPR, FBX4, FIBB, FIBG, FHL1, FLNA, FRMD3, FRIH, FRIL, FUCO, GBRA1, G3P, SYG, GDF15, GELS, GSTP1, HABP2, HGF, 1A68, HMGB1, ROA1, ROA2, HNRPF, HPT, HS90B, ENPL, GRP75, HSPB1, CH60, SIAL, IFT74, IGF1, IGHA2, IL2RB, IL8, IL9, RASK, K1C19, K2C8, LAMA2, LEG3, LMNB1, MARE1, MCM4, MIF, MMP7, MMP9, CD20, MYL6, MYL9, NDKA, NNMT, A1AG1, PCKGM, PDIA3, PDIA6, PDXK, PEBP1, PIPNA, KPYM, UROK, IPYR, PRDX1, KPCD1, PRL, TMG4, PSME3, PTEN, FAK1, FAK2, RBX1, REG4, RHOA, RHOB, RHOC, RSSA, RRBP1, S10AB, S10AC, S10A8, S109, SAM, SAA2, SEGN, SDCG3, DHSA, SBP1, SELPL, SEP9, A1AT, AACT, ILEU, SPB6, SF3B3, SKP1, ADT2, ISK1, SPON2, OSTP, SRC, STK11, HNRPQ, TAL1, TRFE, TSP1, TIMP1, TKT, TSG6, TR10B, TNF6B, P53, TPM2, TCTP, TRAP1, THTR, TBB1, UGDH, UGPA, VEGFA, VILI, VIME, VNN1, 1433Z, CCR5, FUCO and combinations thereof in a biological sample from the subject.

Methods are also disclosed for the diagnosis, prediction, prognosis and/or monitoring a colon disease or colorectal cancer in a subject comprising: measuring at least one biomarker selected from the group SPB6, FRIL, P53, 1A68, ENOA, TKT, and combinations thereof in a biological sample from the subject.

Methods are disclosed for the diagnosis, prediction, prognosis and/or monitoring a colon disease or colorectal cancer in a subject comprising: measuring at least one biomarker selected from the group SPB6, FRIL, P53, 1A68, ENOA, TKT, TSG6, TPM2, ADT2, FHL1, CCR5, CEAM5, SPON2, 1A68, RBX1, COR1C, VIME, PSME3, and combinations thereof in a biological sample from the subject.

Methods are disclosed for the diagnosis, prediction, prognosis and/or monitoring a colon disease or colorectal cancer in a subject comprising: measuring at least one biomarker selected from the group SPB6, FRIL, P53, 1A68, ENOA, TKT, TSG6, TPM2, ADT2, FHL1, CCR5, CEAM5, SPON2, 1A68, RBX1, COR1C, VIME, PSME3, MIC1, STK11, IPYR, SBP1, PEBP1, CATD, HPT, ANXA5, ALDOA, LAMA2, CATZ, ACTB, AACT, and combinations thereof in a biological sample from the subject.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A shows a graph illustrating the predictive performance of a biomarker profile for colon polyps according to Example 3A.

FIG. 1B shows a graph illustrating the predictive performance of a biomarker profile for colon polyps according to Example 3B, with the Y-axis as the average true positive rate, and the X-axis as the false positive rate.

FIG. 2A shows a validation of the testing set performance for Example 3A.

FIG. 2B shows a validation of the testing set performance for Example 3B, with the Y-axis as the average true positive rate, and the X-axis as the false positive rate.

FIG. 3 shows a pareto plot of the feature-frequency table for Example 3A.

FIG. 4 shows a pareto plot of the feature-frequency table for Example 3B, with the Y-axis as the feature occurrence, and the X-axis as the feature rank.

FIG. 5 shows a graph illustrating the predictive performance of a biomarker profile for colon polyps according to Example 3A with a smaller set.

FIG. 6 shows a validation of the testing set performance for Example 3A with a smaller set.

FIG. 7 shows the masses of the 1014 features represented in the classifiers assembled in Example 3A, each present 3 or more times.

FIG. 8 shows the masses of the 206 features represented in the classifiers assembled in Example 3B.

FIG. 9 provides a table of additional biomarkers for inclusion or exclusion.

FIG. 10 shows a graph illustrating the predictive performance of a biomarker profile for CRC according to Example 4, with the Y-axis as the average true positive rate, and the X-axis as the false positive rate.

FIG. 11 shows a pareto plot of the feature-frequency table for assembled in Example 4.

FIG. 12 shows the peptide fragment transitional ions represented in the classifier predictive of CRC assembled in Example 4.

FIG. 13 illustrates an embodiment of various components of a generalized computer system 1300.

FIG. 14 is a diagram illustrating an embodiment of an architecture of a computer system that can be used in connection with embodiments of the present disclosure 1400.

FIG. 15 is a diagram illustrating an embodiment of a computer network that can be used in connection with embodiments of the present disclosure 1500.

FIG. 16 is a diagram illustrating an embodiment of architecture of a computer system that can be used in connection with embodiments of the present disclosure 1600.

### DETAILED DESCRIPTION OF THE DISCLOSURE

### I. Definitions

The term “colorectal cancer status” refers to the status of the disease in subject. Examples of types of colorectal cancer statuses include, but are not limited to, the subject's risk of cancer, including colorectal carcinoma, the presence or absence of disease (e.g., polyp or adenocarcinoma), the stage of disease in a patient (e.g., carcinoma), and the effectiveness of treatment of disease.

The term “mass spectrometer” refers to a gas phase ion spectrometer that measures a parameter that can be translated into mass-to-charge (m/z) ratios of gas phase ions. Mass spectrometers generally include an ion source and a mass analyzer. Examples of mass spectrometers are time-of-flight, magnetic sector, quadrupole filter, ion trap, ion cyclotron resonance, electrostatic sector analyzer and hybrids of these. “Mass spectrometry” refers to the use of a mass spectrometer to detect gas phase ions.

The term “tandem mass spectrometer” refers to any mass spectrometer that is capable of performing two successive stages of m/z-based discrimination or measurement of ions, including ions in an ion mixture. The phrase includes mass spectrometers having two mass analyzers that are capable of performing two successive stages of m/z-based discrimination or measurement of ions tandem-in-space. The phrase further includes mass spectrometers having a single mass analyzer that is capable of performing two successive stages of m/z-based discrimination or measurement of ions tandem-in-time. The phrase thus explicitly includes Qq-TOF mass spectrometers, ion trap mass spectrometers, ion trap-TOF mass spectrometers, TOF-TOF mass spectrometers, Fourier transform ion cyclotron resonance mass spectrometers, electrostatic sector-magnetic sector mass spectrometers, and combinations thereof.

The term “biochip” refers to a solid substrate having a generally planar surface to which an adsorbent is attached. Frequently, the surface of the biochip comprises a plurality of addressable locations, each of which location has the adsorbent bound there. Biochips can be adapted to engage a probe interface, and therefore, function as probes. Protein biochips are adapted for the capture of polypeptides and can be comprise surfaces having chromatographic or biospecific adsorbents attached thereto at addressable locations. Microaaray chips are generally used for DNA and RNA gene expression detection.

The term “biomarker” refers to a polypeptide (of a particular apparent molecular weight), which is differentially present in a sample taken from subjects having human colorectal cancer as compared to a comparable sample taken from control subjects (e.g., a person with a negative diagnosis or undetectable colorectal cancer, normal or healthy subject, or, for example, from the same individual at a different time point). The term “biomarker” is used interchangeably with the term “marker”. A biomarker can be a gene, such DNA or RNA or a genetic variation of the DNA or RNA, their binding partners, splice-variants. A biomarker can be a protein or protein fragment or transitional ion of an amino acid sequence, or one or more modifications on a protein amino acid sequence. In addition, a protein biomarker can be a binding partner of a protein or protein fragment or transitional ion of an amino acid sequence.

The terms “polypeptide,” “peptide” and “protein” are used interchangeably herein to refer to a polymer of amino acid residues. A polypeptide is a single linear polymer chain of amino acids bonded together by peptide bonds between the carboxyl and amino groups of adjacent amino acid residues. Polypeptides can be modified, e.g., by the addition of carbohydrate, phosphorylation, ect.

The term “immunoassay” is an assay that uses an antibody to specifically bind an antigen (e.g., a marker). The immunoassay is characterized by the use of specific binding properties of a particular antibody to isolate, target, and/or quantify the antigen.

The term “antibody” refers to a polypeptide ligand substantially encoded by an immunoglobulin gene or immunoglobulin genes, or fragments thereof, which specifically binds and recognizes an epitope. Antibodies exist, e.g., as intact immunoglobulins or as a number of well-characterized fragments produced by digestion with various peptidases. This includes, e.g., Fab″ and F(ab)″₂ fragments. As used herein, the term “antibody” also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, but does not include the heavy chain variable region.

The term “tumor” refers to a solid or fluid-filled lesion that may be formed by cancerous or non-cancerous cells. The terms “mass” and “nodule” are often used synonymously with “tumor”. Tumors include malignant tumors or benign tumors. An example of a malignant tumor can be a carcinoma which is known to comprise transformed cells.

The term “polyp” refers to an abnormal growth of tissue projecting from a mucous membrane. If it is attached to the surface by a narrow elongated stalk, it is said to be pedunculated polyp. If no stalk is present, it is said to be sessile polyp. Polyps may be malignant, pre-cancerous, or benign. Polyps may be removed by various procedures, such as surgery, or for example, during colonoscopy with polypectomy.

The term “adenomatous polyps” or “adenomas” are used interchangeably herein to refer to polyps that grow on the lining of the colon and which carry an increased risk of cancer. The adenomatous polyp is considered pre-malignant; however, some are likely to develop into colon cancer. Tubular adenomas are the most common of the adenomatous polyps and they are the least likely of colon polyps to develop into colon cancer. Tubulovillous adenoma is yet another type. Villous adenomas area third type that is normally larger in size than the other two types of adenomas and they are associated with the highest morbidity and mortality rates of all polyps.

The term “binding partners” refers to pairs of molecules, typically pairs of biomolecules that exhibit specific binding. Protein-protein interactions which can occur between two or more proteins, when bound together they often to carry out their biological function. Interactions between proteins are important for the majority of biological functions. For example, signals from the exterior of a cell are mediated via ligand and receptor proteins to the inside of that cell by protein-protein interactions of the signaling molecules. For example, molecular binding partners include, without limitation, receptor and ligand, antibody and antigen, biotin and avidin, and others.

The term “control reference” refers to a known steady state molecule or a non-diseased, healthy condition that is used as relative marker in which to study the fluctuations or compare the non-steady state molecules or normal non-diseased healthy condition, or it can also be used to calibrate or normalize values. In various embodiments, a control reference value is a calculated value from a combination of factors or a combination of a range of factors, such as a combination of biomarker concentrations or a combination of ranges of concentrations.

The term “subject,” “individual” or “patient” is used interchangeably herein, which refers to a vertebrate, preferably a mammal, more preferably a human. Mammals include, but are not limited to, murines, simians, farm animals, sport animals, and pets. Specific mammals include rats, mice, cats, dogs, monkeys, and humans. Non-human mammals include all mammals other than humans. Tissues, cells and their progeny of a biological entity obtained in vitro or cultured in vitro are also encompassed.

The term “in vivo” refers to an event that takes place in a subject's body.

The term “in vitro” refers to an event that takes places outside of a subject's body. For example, an in vitro assay encompasses any assay run outside of a subject assay. In vitro assays encompass cell-based assays in which cells alive or dead are employed. In vitro assays also encompass a cell-free assay in which no intact cells are employed.

The term “measuring” means methods which include detecting the presence or absence of marker(s) in the sample, quantifying the amount of marker(s) in the sample, and/or qualifying the type of biomarker. Measuring can be accomplished by methods known in the art and those further described herein, including but not limited to mass spectrometry approaches and immunoassay approaches or any suitable methods can be used to detect and measure one or more of the markers described herein.

The term “detect” refers to identifying the presence, absence or amount of the object to be detected. Non-limiting examples include, but are not limited to, detection of a DNA molecules, proteins, peptides, protein complexes, RNA molecules or metabolites.

The term “differentially present” refers to differences in the quantity and/or the frequency of a marker present in a sample taken from subjects as compared to a control reference or a control non-diseased, healthy subject. A marker can be differentially present in terms of quantity, frequency or both.

The term “monitoring” refers to recording changes in a continuously varying parameter.

The term “diagnostic” or “diagnosis” is used interchangeably herein means identifying the presence or nature of a pathologic condition, or subtype of a pathologic condition, i.e., presence or risk of colon polyps. Diagnostic methods differ in their sensitivity and specificity. Diagnostic methods may not provide a definitive diagnosis of a condition; however, it suffices if the method provides a positive indication that aids in diagnosis.

The term “prognosis” is used herein to refer to the prediction of the likelihood of disease or diseases progression, including recurrence and therapeutic response.

The term “prediction” is used herein to refer to the likelihood that a patient will have a particular clinical outcome, whether positive or negative. The predictive methods of the present disclosure can be used clinically to make treatment decisions by choosing the most appropriate treatment modalities for any particular patient.

The term “report” refers to a printed result provided from the methods of the present to physician is inconclusive or confirmatory as necessary. The report could indicate presence of, nature of, or risk for the pathological condition. The report can also indicate what treatment is most appropriate; e.g., no action, surgery, further tests, or administering therapeutic agents.

### II. General Overview

The development of biomarker profiles for diagnostics, prognostics, and predicted drug responses for disease can be useful to the medical community.

The present disclosure provides for methods, compositions, systems, and kits that analyze a complex biological sample from an individual using various assays coupled with algorithms executed by a processor instructed by computer readable medium for determining a biomarker, which is indicative for worsening or improving in clinical status or health. Generally, the methods use various molecules from multiple levels of molecular biology, e.g., the polynucleotide (DNA or RNA), polypeptide, and metabolite levels, of the biological system to identify a biomarker or biomarker profile of a disease such as colon cancer, colon polyp, and various colorectal diseases are contemplated.

The present disclosure also provides biomarkers and systems useful for the diagnosis, prediction, prognosis, or monitoring for the presence or recovery from colon polyp or colon cancer in an individual.

The present disclosure also provides a commercial diagnostic kit that in general will include compositions used for the detection of biomarkers provided herein, instructions, and a report that indicates the diagnosis, prediction, prognosis, presence or recovery from colon polyp or colon cancer in an individual. Clinical predictions or status provided by the report can indicate a likelihood, chance or risk that a subject will develop clinically manifest colon polyp and colon cancer, for example within a certain time period or at a given age in individual not having yet clinically presented a colon polyp or carcinoma.

### III. Methods

The present disclosure provides medical diagnostic methods based on proteomic and/or genomic patterns, using data obtained by mass spectrometry. The method allows classifying the patients as to their disease stage based on their proteomic and/or genomic patterns.

Colorectal cancer, also known as colon cancer, rectal cancer, or bowel cancer, is a cancer from uncontrolled cell growth in the colon or rectum. Additionally, the present disclosure provides new biomarkers for medical diagnosis of colon polyp and colorectal cancer.

A colon polyp is benign clump of cells that forms on the lining of the large intestine or colon. Almost all polyps are initially non-malignant. However, over time some can turn into cancerous lesions. The cause of most colon polyps is not known, but they are common in adults. Since colon polyps are asymptomatic, regular screening for colon polyps is recommended. Currently, the methods used for screening for polyps are highly invasive and expensive. Thus, despite the benefit of colonoscopy screening in the prevention and reduction of colon cancer, many of the people for whom the procedure is recommended decline to undertake it, primarily due to concerns about cost, discomfort, and adverse events. This group represents tens of millions of people in the U.S. alone.

A molecular test which helps classify the likelihood that a patient has a higher risk for the presence of a colon polyp, adenoma, or a cancerous tumor such as, carcinoma may help physicians to guide patients' attitudes and actions regarding reluctance to undergo colonoscopy. Increased colonoscopy screening compliance would result in early detection of cancer or pre-cancerous adenoma and a reduction in colon cancer-related morbidity and mortality.

The present disclosure provides for a protein biomarker test which is less invasive than a colonoscopy, and that will determine an individual's protein expression fingerprint or profile. In some applications of the disclosure, a report is generated based on the predicted likelihood an individual's polyp status and/or risk of developing colon polyps or colon cancer. Thus, the present disclosure provides methods, kits, compositions, and systems that provide information for an individual's colon polyp status and/or risk of developing colon polyps, or colon cancer.

In one aspect of the disclosure, a set of protein-based classifiers (e.g. biomarker profile) have been identified by an LCMS-based procedure which enable prediction of colonoscopy procedure outcomes with respect to the presence or absence of colon polyps, adenomas or carcinomas in the patients.

In one aspect of the disclosure, an LCMS-based approach has been used to identify plasma-protein-based molecular features that can comprise one or more classifiers that discriminate patients who are more likely to have polyps, adenomas, or tumors.

In one aspect of the disclosure, classifiers are used to determine which individuals are not likely to have polyps, adenomas, or tumors, and who therefore might not need to have a colonoscopy.

In one aspect of the disclosure, classifiers are used to measure the completeness of suspicious polyp removal during colonoscopy by comparing classifier values before and after the procedure.

In one aspect of the disclosure, classifiers are used during intervals between regular screening colonoscopies to catch so-called interval disease.

In one aspect of the disclosure, classifiers are used to increase the time between successive colonoscopies in patients with an elevated risk profile. Examples of patients with an elevated risk profile can include patients with previous polypectomy or other pathology.

The disclosure provides a method of generating and analysing a blood protein fragmentation profile, in terms of the size, and sequence of particular fragments derived from intact proteins together with the position where enzymes scission occurs (e.g. trypsin digestion, ect.) along the full protein polypeptide chain is characteristic of the diseased state of the colon.

It is completed that the method, kits, compositions, and systems provided by the present disclosure may also be automated in whole or in part depending upon the application.

A. Algorithm-Based Methods

The present disclosure provides an algorithm-based diagnostic assay for predicting a clinical outcome for a patient with colon polyps or colon cancer. The expression level of one or more protein biomarkers may be used alone or arranged into functional subsets to calculate a quantitative score that can be used to predict the likelihood of a clinical outcome.

A “biomarker” or “maker” of the present disclosure can be a polypeptide of a particular apparent molecular weight, a gene, such DNA or RNA or a genetic variation of the DNA or RNA, their binding partners, splice-variants. A biomarker can be a protein or protein fragment or transitional ion of an amino acid sequence, or one or more modifications on a protein amino acid sequence. In addition, a protein biomarker can be a binding partner of a protein or protein fragment or transitional ion of an amino acid sequence.

The algorithm-based assay and associated information provided by the practice of the methods of the present disclosure facilitate optimal treatment decision-making in patients presenting with colon tumors. For example, such a clinical tool would enable physicians to identify patients who have a low likelihood of having a polyp or carcinoma and therefore would not need anti-cancer treatment, or who have a high likelihood of having an aggressive cancer and therefore would need anti-cancer treatment.

A quantitative score may be determined by the application of a specific algorithm. The algorithm used to calculate the quantitative score in the methods disclosed herein may group the expression level values of a biomarker or groups of biomarkers. The formation of a particular group of biomarkers, in addition, can facilitate the mathematical weighting of the contribution of various expression levels of biomarker or biomarker subsets (e.g. classifier) to the quantitative score. The present disclosure provides a various algorithms for calculating the quantitative scores.

B. Normalization of Data

The expression data used in the methods disclosed herein can be normalized. Normalization refers to a process to correct for example, differences in the amount of genes or protein levels assayed and variability in the quality of the template used, to remove unwanted sources of systematic variation measurements involved in the processing and detection of genes or protein expression. Other sources of systematic variation are attributable to laboratory processing conditions.

In some instances, normalization methods can be used for the normalization of laboratory processing conditions. Non-limiting examples of normalization of laboratory processing that may be used with methods of the disclosure include but are not limited to: accounting for systematic differences between the instruments, reagents, and equipment used during the data generation process, and/or the date and time or lapse of time in the data collection.

Assays can provide for normalization by incorporating the expression of certain normalizing standard genes or proteins, which do not significantly differ in expression levels under the relevant conditions, that is to say they are known to have a stabilized and consistent expression level in that particular sample type. Suitable normalization genes and proteins that can be used with the present disclosure include housekeeping genes. (See, e.g., E. Eisenberg, et al., Trends in Genetics 19(7):362-365 (2003). In some applications, the normalizing biomarkers (genes and proteins), also referred to as reference genes, known not to exhibit meaningfully different expression levels in colon polyps or cancer as compared to patients with no colon polyps. In some applications, it may be useful to add a stable isotope labeled standards which can be used and represent an entity with known properties for use in data normalization. In other applications, a standard, fixed sample can be measured with each analytical batch to account for instrument and day-to-day measurement variability.

In some applications, diagnostic, prognostic and predictive genes may be normalized relative to the mean of at least 2, 3, 4, 5, 6, 7, 8, 9, 10, 15, 20, 25, 30, 40, or 50 or more reference genes and proteins. Normalization can be based on the mean or median signal of all of the assayed biomarkers or by a global biomarker normalization approach. Those skilled in the art will recognize that normalization may be achieved in numerous ways, and the techniques described above are intended only to be exemplary.

C. Standardization of Data

The expression data used in the methods disclosed herein can be standardized. Standardization refers to a process to effectively put all the genes on a comparable scale. This is performed because some genes will exhibit more variation (a broader range of expression) than others. Standardization is performed by dividing each expression value by its standard deviation across all samples for that gene or protein.

D. Clinical Outcome Score

The use of machine learning algorithms for sub-selecting discriminating biomarkers and for building classification models can be used to determine clinical outcome scores. These algorithms include, but are not limited to, elastic networks, random forests, support vector machines, and logistic regression. These algorithms can hone in on important biomarker features and transform the underlying measurements into score or probability relating to, for example, clinical outcome, disease risk, treatment response, and/or classification of disease status.

In some applications, an increase in the quantitative score indicates an increased likelihood of a poor clinical outcome, good clinical outcome, high risk of disease, low risk of disease, complete response, partial response, stable disease, non-response, and recommended treatments for disease management. In some applications, a decrease in the quantitative score indicates an increased likelihood of a poor clinical outcome, good clinical outcome, high risk of disease, low risk of disease, complete response, partial response, stable disease, non-response, and recommended treatments for disease management.

In some applications, a similar biomarker profile from a patient to a reference profile indicates an increased likelihood of a poor clinical outcome, good clinical outcome, high risk of disease, low risk of disease, complete response, partial response, stable disease, non-response, and recommended treatments for disease management. In some applications, a dissimilar biomarker profile from a patient to a reference profile indicates an increased likelihood of a poor clinical outcome, good clinical outcome, high risk of disease, low risk of disease, complete response, partial response, stable disease, non-response, and recommended treatments for disease management.

In some applications, an increase in one or more biomarker threshold values indicates an increased likelihood of a poor clinical outcome, good clinical outcome, high risk of disease, low risk of disease, complete response, partial response, stable disease, non-response, and recommended treatments for disease management. In some applications, a decrease in one or more biomarker threshold values indicates an increased likelihood of a poor clinical outcome, good clinical outcome, high risk of disease, low risk of disease, complete response, partial response, stable disease, non-response, and recommended treatments for disease management.

In some applications, an increase in quantitative score, one or more biomarker threshold, a similar biomarker profile values or combinations thereof indicates an increased likelihood of a poor clinical outcome, good clinical outcome, high risk of disease, low risk of disease, complete response, partial response, stable disease, non-response, and recommended treatments for disease management. In some applications, an decrease in quantitative score, one or more biomarker threshold, a similar biomarker profile values or combinations thereof indicates an increased likelihood of a poor clinical outcome, good clinical outcome, high risk of disease, low risk of disease, complete response, partial response, stable disease, non-response, and recommended treatments for disease management.

E. Sample Preparation and Processing

Before analyzing the sample it may be desirable to perform one or more sample preparation operations upon the sample. Generally, these sample preparation operations may include such manipulations as extraction and isolation of intracellular material from a cell or tissue such as, the extraction of nucleic acids, protein, or other macromolecules from the samples.

Sample preparation which can be used with the methods of disclosure include but are not limited to, centrifugation, affinity chromatography, magnetic separation, immunoassay, nucleic acid assay, receptor-based assay, cytometric assay, colorimetric assay, enzymatic assay, electrophoretic assay, electrochemical assay, spectroscopic assay, chromatographic assay, microscopic assay, topographic assay, calorimetric assay, radioisotope assay, protein synthesis assay, histological assay, culture assay, and combinations thereof.

Sample preparation can further include dilution by an appropriate solvent and amount to ensure the appropriate range of concentration level is detected by a given assay.

Accessing the nucleic acids and macromolecules from the intercellular space of the sample may generally be performed by either physical, chemical methods, or a combination of both. In some applications of the methods, following the isolation of the crude extract, it will often be desirable to separate the nucleic acids, proteins, cell membrane particles, and the like. In some applications of the methods it will be desirable to keep the nucleic acids with its proteins, and cell membrane particles.

In some applications of the methods provided herein, nucleic acids and proteins can be extracted from a biological sample prior to analysis using methods of the disclosure. Extraction can be by means including, but not limited to, the use of detergent lysates, sonication, or vortexing with glass beads.

In some applications, molecules can be isolated using any technique suitable in the art including, but not limited to, techniques using gradient centrifugation (e.g., cesium chloride gradients, sucrose gradients, glucose gradients, etc.), centrifugation protocols, boiling, purification kits, and the use of liquid extraction with agent extraction methods such as methods using Trizol or DNAzol.

Samples may be prepared according to standard biological sample preparation depending on the desired detection method. For example for mass spectrometry detection, biological samples obtained from a patient may be centrifigued, filtered, processed by immunoaffinity column, separated into fractions, partially digested, and combinations thereof. Various fractions may be resuspended in appropriate carrier such as buffer or other type of loading solution for detection and analysis, including LCMS loading buffer.

F. Methods of Detection

The present disclosure provides for methods for detecting biomarkers in biological samples. Biomarkers can include but are not limited to proteins, metabolites, DNA molecules, and RNA molecules. More specifically the present disclosure is based on the discovery of protein biomarkers that are differentially expressed in subjects that have a colon polyp, or are likely to develop colon polyps. Therefore the detection of one or more of these differentially expressed biomarkers in a biological sample provides useful information whether or not a subject is at risk or suffering from colon polyps and what type of nature or state of the condition. Any suitable method can be used to detect one or more of the biomarker described herein.

Useful analyte capture agents that can be used with the present disclosure include but are not limited to antibodies, such as crude serum containing antibodies, purified antibodies, monoclonal antibodies, polyclonal antibodies, synthetic antibodies, antibody fragments (for example, Fab fragments); antibody interacting agents, such as protein A, carbohydrate binding proteins, and other interactants; protein interactants (for example avidin and its derivatives); peptides; and small chemical entities, such as enzyme substrates, cofactors, metal ions/chelates, and haptens. Antibodies may be modified or chemically treated to optimize binding to targets or solid surfaces (e.g. biochips and columns).

In one aspect of the disclosure the biomarker can be detected in a biological sample using an immunoassay. Immunoassays are assay that use an antibody that specifically bind to or recognizes an antigen (e.g. site on a protein or peptide, biomarker target). The method includes the steps of contacting the biological sample with the antibody and allowing the antibody to form a complex of with the antigen in the sample, washing the sample and detecting the antibody-antigen complex with a detection reagent. In one embodiment, antibodies that recognize the biomarkers may be commercially available. In another embodiment, an antibody that recognizes the biomarkers may be generated by known methods of antibody production.

Alternatively, the marker in the sample can be detected using an indirect assay, wherein, for example, a second, labeled antibody is used to detect bound marker-specific antibody. Exemplary detectable labels include magnetic beads (e.g., DYNABEADS™), fluorescent dyes, radiolabels, enzymes (e.g., horse radish peroxide, alkaline phosphatase and others commonly used), and calorimetric labels such as colloidal gold or colored glass or plastic beads. The marker in the sample can be detected using and/or in a competition or inhibition assay wherein, for example, a monoclonal antibody which binds to a distinct epitope of the marker is incubated simultaneously with the mixture.

The conditions to detect an antigen using an immunoassay will be dependent on the particular antibody used. Also, the incubation time will depend upon the assay format, marker, volume of solution, concentrations and the like. In general, the immunoassays will be carried out at room temperature, although they can be conducted over a range of temperatures, such as 10.degrees. to 40 degrees Celsius depending on the antibody used.

There are various types of immunoassay known in the art that as a starting basis can be used to tailor the assay for the detection of the biomarkers of the present disclosure. Useful assays can include, for example, an enzyme immune assay (EIA) such as enzyme-linked immunosorbent assay (ELISA). There are many variants of these approaches, but those are based on a similar idea. For example, if an antigen can be bound to a solid support or surface, it can be detected by reacting it with a specific antibody and the antibody can be quantitated by reacting it with either a secondary antibody or by incorporating a label directly into the primary antibody. Alternatively, an antibody can be bound to a solid surface and the antigen added. A second antibody that recognizes a distinct epitope on the antigen can then be added and detected. This is frequently called a ‘sandwich assay’ and can frequently be used to avoid problems of high background or non-specific reactions. These types of assays are sensitive and reproducible enough to measure low concentrations of antigens in a biological sample.

Immunoassays can be used to determine presence or absence of a marker in a sample as well as the quantity of a marker in a sample. Methods for measuring the amount of, or presence of, antibody-marker complex include but are not limited to, fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). In general these regents are used with optical detection methods, such as various forms of microscopy, imaging methods and non-imaging methods. Electrochemical methods include voltametry and amperometry methods. Radio frequency methods include multipolar resonance spectroscopy.

In one aspect, the disclosure can use antibodies for the detection of the biomarkers. Antibodies can be made that specifically bind to the biomarkers of the present assay can be prepared using standard methods known in the art. For example polyclonal antibodies can be produced by injecting an antigen into a mammal, such as a mouse, rat, rabbit, goat, sheep, or horse for large quantities of antibody. Blood isolated from these animals contains polyclonal antibodies—multiple antibodies that bind to the same antigen. Alternatively polyclonal antibodies can be produced by injecting the antigen into chickens for generation of polyclonal antibodies in egg yolk. In addition, antibodies can be made that specifically recognize modified forms for the biomarkers such as a phosphorylated form of the biomarker, that is to say, they will recognize a tyrosine or a serine after phosphorylation, but not in the absence of phosphate. In this way antibodies can be used to determine the phosphorylation state of a particular biomarker.

Antibodies can be obtained commercially or produced using well-established methods. To obtain antibody that is specific for a single epitope of an antigen, antibody-secreting lymphocytes are isolated from the animal and immortalized by fusing them with a cancer cell line. The fused cells are called hybridomas, and will continually grow and secrete antibody in culture. Single hybridoma cells are isolated by dilution cloning to generate cell clones that all produce the same antibody; these antibodies are called monoclonal antibodies.

Polyclonal and monoclonal antibodies can be purified in several ways. For example, one can isolate an antibody using antigen-affinity chromatography which is couple to bacterial proteins such as Protein A, Protein G, Protein L or the recombinant fusion protein, Protein A/G followed by detection of via UV light at 280 nm absorbance of the eluate fractions to determine which fractions contain the antibody. Protein A/G binds to all subclasses of human IgG, making it useful for purifying polyclonal or monoclonal IgG antibodies whose subclasses have not been determined. In addition, it binds to IgA, IgE, IgM and (to a lesser extent) IgD. Protein A/G also binds to all subclasses of mouse IgG but does not bind mouse IgA, IgM or serum albumin. This feature, allows Protein A/G to be used for purification and detection of mouse monoclonal IgG antibodies, without interference from IgA, IgM and serum albumin.

Antibodies can be derived from different classes or isotypes of molecules such as, for example, IgA, IgA IgD, IgE, IgM and IgG. The IgA are designed for secretion in the bodily fluids while others, like the IgM are designed to be expressed on the cell surface. The antibody that is most useful in biological studies is the IgG class, a protein molecule that is made and secreted and can recognize specific antigens. The IgG is composed of two subunits including two “heavy” chains and two “light” chains. These are assembled in a symmetrical structure and each IgG has two identical antigen recognition domains. The antigen recognition domain is a combination of amino acids from both the heavy and light chains. The molecule is roughly shaped like a “Y” and the arms/tips of the molecule comprise the antigen-recognizing regions or Fab (fragment, antigen binding) region, while the stem of Fc (Fragment, crystallizable) region is not involved in recognition and is fairly constant. The constant region is identical in all antibodies of the same isotype, but differs in antibodies of different isotypes.

It is also possible to use an antibody to detect a protein after fractionation by western blotting. In one aspect, the disclosure can use western blotting for the detection of the biomarkers. Western blot (protein immunoblot) is an analytical technique used to detect specific proteins in the given sample or protein extract from a sample. It uses gel electrophoresis, SDS-PAGE to separate either native proteins by their 3-dimensional structure or it can be ran under denaturing conditions to separate proteins by their length. After separation by gel electrophoresis, the proteins are then transferred to a membrane (typically nitrocellulose or PVDF). The proteins transferred from the SDS-PAGE to a membrane can then be incubated with particular antibodies under gentle agitation, rinsed to remove non-specific binding and the protein-antibody complex bound to the blot can be detected using either a one-step or two step detection methods. The one step method includes a probe antibody which both recognizes the protein of interest and contains a detectable label, probes which are often available for known protein tags. The two-step detection method involves a secondary antibody that has a reporter enzyme or reporter bound to it. With appropriate reference controls, this approach can be used to measure the abundance of a protein.

In one aspect, the method of the disclosure can use flow cytometry. Flow cytometry is a laser based, biophysical technology that can be used for biomarker detection, quantification (cell counting) and cell isolation. This technology is routinely used in the diagnosis of health disorders, especially blood cancers. In general, flow cytometry works by suspending single cells in a stream of fluid, a beam of light (usually laser light) of a single wavelength is directed onto the stream of liquid, and the scatter light caused by the passing cell is detected by a electronic detection apparatus. Fluorescence-activated cell sorting (FACS) is a specialized type of flow cytometry that often uses the aid of florescent-labeled antibodies to detect antigens on cell of interest. This additional feature of antibody labeling use in FACS provides for simultaneous multiparametric analysis and quantification based upon the specific light scattering and fluorescent characteristics of each cell florescent-labeled cell and it provides physical separation of the population of cells of interest as well as traditional flow cytometry does.

A wide range of fluorophores can be used as labels in flow cytometry. Fluorophores are typically attached to an antibody that recognizes a target feature on or in the cell. Examples of suitable fluorescent labels include, but are not limited to: fluorescein (FITC), 5,6-carboxymethyl fluorescein, Texas red, nitrobenz-2-oxa-1,3-diazol-4-yl (NBD), and the cyanine dyes Cy3, Cy3.5, Cy5, Cy5.5 and Cy7. Other Fluorescent labels such as Alexa Fluor® dyes, DNA content dye such as DAPI, Hoechst dyes are well known in the art and all can be easily obtained from a variety of commercial sources. Each fluorophore has a characteristic peak excitation and emission wavelength, and the emission spectra often overlap. The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus choosing one that do not have a lot of spectra overlap allows their simultaneous detection. The fluorescent labels can be obtained from a variety of commercial sources. The maximum number of distinguishable fluorescent labels is thought to be around approximately 17 or 18 different fluorescent labels. This level of complex read-out necessitates laborious optimization to limit artifacts, as well as complex deconvolution algorithms to separate overlapping spectra. Quantum dots are sometimes used in place of traditional fluorophores because of their narrower emission peaks. Other methods that can be used for detecting include isotope labeled antibodies, such as lanthanide isotopes. However this technology ultimately destroys the cells, precluding their recovery for further analysis.

In one aspect, the method of the disclosure can use immunohistochemistry for detecting the expression levels of the biomarkers of the present disclosure. Thus, antibodies specific for each marker are used to detect expression of the claimed biomarkers in a tissue sample. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols are well known in the art and protocols and antibodies are commercially available. Alternatively, one could make an antibody to the biomarkers or modified versions of the biomarker or binding partners as disclosure herein that would be useful for determining the expression levels of in a tissue sample.

In one aspect, the method of the disclosure can use a biochip. Biochips can be used to screen a large number of macromolecules. In this technology macromolecules are attached to the surface of the biochip in an ordered array format. The grid pattern of the test regions allowed analysed by imaging software to rapidly and simultaneously quantify the individual analytes at their predetermined locations (addresses). The CCD camera is a sensitive and high-resolution sensor able to accurately detect and quantify very low levels of light on the chip.

Biochips can be designed with immobilized nucleic acid molecules, full-length proteins, antibodies, affibodies (small molecules engineered to mimic monoclonal antibodies), aptamers (nucleic acid-based ligands) or chemical compounds. A chip could be designed to detect multiple macromolecule types on one chip. For example, a chip could be designed to detect nucleic acid molecules, proteins and metabolites on one chip. The biochip is used to and designed to simultaneously analyze a panel biomarker in a single sample, producing a subjects profile for these biomarkers. The use of the biochip allows for the multiple analyses to be performed reducing the overall processing time and the amount of sample required.

Protein microarray are a particular type of biochip which can be used with the present disclosure. The chip consists of a support surface such as a glass slide, nitrocellulose membrane, bead, or microtitre plate, to which an array of capture proteins are bound in an arrayed format onto a solid surface. Protein array detection methods must give a high signal and a low background. Detection probe molecules, typically labeled with a fluorescent dye, are added to the array. Any reaction between the probe and the immobilized protein emits a fluorescent signal that is read by a laser scanner. Such protein microarrays are rapid, automated, and offer high sensitivity of protein biomarker read-outs for diagnostic tests. However, it would be immediately appreciated to those skilled in the art that they are a variety of detection methods that can be used with this technology.

There are at least three types of protein microarrays that are currently used to study the biochemical activities of proteins. For example there are analytical microarrays (also known as capture arrays), Functional protein microarrays (also known as target protein arrays) and Reverse phase protein microarray (RPA).

The present disclosure provides for the detection of the biomarkers using an analytical protein microarray. Analytical protein microarrays are constructed using a library of antibodies, aptamers or affibodies. The array is probed with a complex protein solution such as a blood, serum or a cell lysate that function by capturing protein molecules they specifically bind to. Analysis of the resulting binding reactions using various detection systems can provide information about expression levels of particular proteins in the sample as well as measurements of binding affinities and specificities. This type of protein microarray is especially useful in comparing protein expression in different samples.

In one aspect, the method of the disclosure can use functional protein microarrays are constructed by immobilising large numbers of purified full-length functional proteins or protein domains and are used to identify protein-protein, protein-DNA, protein-RNA, protein-phospholipid, and protein-small molecule interactions, to assay enzymatic activity and to detect antibodies and demonstrate their specificity. These protein microarray biochips can be used to study the biochemical activities of the entire proteome in a sample.

In one aspect, the method of the disclosure can use reverse phase protein microarray (RPA). Reverse phase protein microarray are constructed from tissue and cell lysates that are arrayed onto the microarray and probed with antibodies against the target protein of interest. These antibodies are typically detected with chemiluminescent, fluorescent or colorimetric assays. In addition to the protein in the lysate, reference control peptides are printed on the slides to allow for protein quantification. RPAs allow for the determination of the presence of altered proteins or other agents that may be the result of disease and present in a diseased cell.

The present disclosure provides for the detection of the biomarkers using mass spectroscopy (alternatively referred to as mass spectrometry). Mass spectrometry (MS) is an analytical technique that measures the mass-to-charge ratio of charged particles. It is primarily used for determining the elemental composition of a sample or molecule, and for elucidating the chemical structures of molecules, such as peptides and other chemical compounds. MS works by ionizing chemical compounds to generate charged molecules or molecule fragments and measuring their mass-to-charge ratios MS instruments typically consist of three modules (1) an ion source, which can convert gas phase sample molecules into ions (or, in the case of electrospray ionization, move ions that exist in solution into the gas phase) (2) a mass analyzer, which sorts the ions by their masses by applying electromagnetic fields and (3) detector, which measures the value of an indicator quantity and thus provides data for calculating the abundances of each ion present.

Suitable mass spectrometry methods to be used with the present disclosure include but are not limited to, one or more of electrospray ionization mass spectrometry (ESI-MS), ESI-MS/MS, ESI-MS/(MS)ₙ, matrix-assisted laser desorption ionization time-of-flight mass spectrometry (MALDI-TOF-MS), surface-enhanced laser desorption/ionization time-of-flight mass spectrometry (SELDI-TOF-MS), tandem liquid chromatography-mass spectrometry (LC-MS/MS) mass spectrometry, desorption/ionization on silicon (DIOS), secondary ion mass spectrometry (SIMS), quadrupole time-of-flight (Q-TOF), atmospheric pressure chemical ionization mass spectrometry (APCI-MS), APCI-MS/MS, APCI-(MS), atmospheric pressure photoionization mass spectrometry (APPI-MS), APPI-MS/MS, and APPI-(MS)ₙ, quadrupole mass spectrometry, Fourier transform mass spectrometry (FTMS), and ion trap mass spectrometry, where n is an integer greater than zero.

To gain insight into the underlying proteomics of a sample, LC-MS is commonly used to resolve the components of a complex mixture. LC-MS method generally involves protease digestion and denaturation (usually involving a protease, such as trypsin and a denaturant such as, urea to denature tertiary structure and iodoacetamide to cap cysteine residues) followed by LC-MS with peptide mass fingerprinting or LC-MS/MS (tandem MS) to derive sequence of individual peptides. LC-MS/MS is most commonly used for proteomic analysis of complex samples where peptide masses may overlap even with a high-resolution mass spectrometer. Samples of complex biological fluids like human serum may be first separated on an SDS-PAGE gel or HPLC-SCX and then run in LC-MS/MS allowing for the identification of over 1000 proteins.

While multiple mass spectrometric approaches can be used with the methods of the disclosure as provided herein, in some applications it may be desired to quantify proteins in biological samples from a selected subset of proteins of interest. One such MS technique that can be used with the present disclosure is Multiple Reaction Monitoring Mass Spectrometry (MRM-MS), or alternatively referred to as Selected Reaction Monitoring Mass Spectrometry (SRM-MS).

The MRM-MS technique uses a triple quadrupole (QQQ) mass spectrometer to select a positively charged ion from the peptide of interest, fragment the positively charged ion and then measure the abundance of a selected positively charged fragment ion. This measurement is commonly referred to as a transition. For example of transition obtained from the method see (TABLE 1).

In some applications the MRM-MS is coupled with High-Pressure Liquid Chromatography (HPLC) and more recently Ultra High-Pressure Liquid Chromatography (UHPLC). In other applications MRM-MS is coupled with UHPLC with a QQQ mass spectrometer to make the desired LC-MS transition measurements for all of the peptides and proteins of interest.

In some applications the utilization of a quadrupole time-of-flight (qTOF) mass spectrometer, time-of-flight time-of-flight (TOF-TOF) mass spectrometer, Orbitrap mass spectrometer, quadrupole Orbitrap mass spectrometer or any Quadrupolar Ion Trap mass spectrometer can be used to select for a positively charged ion from one or more peptides of interest. The fragmented, positively charged ions can then be measured to determine the abundance of a positively charged ion for the quantitation of the peptide or protein of interest.

In some applications the utilization of a time-of-flight (TOF), quadrupole time-of-flight (qTOF) mass spectrometer, time-of-flight time-of-flight (TOF-TOF) mass spectrometer, Orbitrap mass spectrometer or quadrupole Orbitrap mass spectrometer can be used to measure the mass and abundance of a positively charged peptide ion from the protein of interest without fragmentation for quantitation. In this application, the accuracy of the analyte mass measurement can be used as selection criteria of the assay. An isotopically labeled internal standard of a known composition and concentration can be used as part of the mass spectrometric quantitation methodology.

In some applications, time-of-flight (TOF), quadrupole time-of-flight (qTOF) mass spectrometer, time-of-flight time-of-flight (TOF-TOF) mass spectrometer, Orbitrap mass spectrometer or quadrupole Orbitrap mass spectrometer can be used to measure the mass and abundance of a protein of interest for quantitation. In this application, the accuracy of the analyte mass measurement can be used as selection criteria of the assay. Optionally this application can use proteolytic digestion of the protein prior to analysis by mass spectrometry. An isotopically labeled internal standard of a known composition and concentration can be used as part of the mass spectrometric quantitation methodology.

In some applications, various ionization techniques can be coupled to the mass spectrometers provide herein to generate the desired information. Non-limiting exemplary ionization techniques that can be used with the present disclosure include but are not limited to Matrix Assisted Laser Desorption Ionization (MALDI), Desorption Electrospray Ionization (DESI), Direct Assisted Real Time (DART), Surface Assisted Laser Desorption Ionization (SALDI), or Electrospray Ionization (ESI).

In some applications, HPLC and UHPLC can be coupled to a mass spectrometer a number of other peptide and protein separation techniques can be performed prior to mass spectrometric analysis. Some exemplary separation techniques which can be used for separation of the desired analyte (e.g., peptide or protein) from the matrix background include but are not limited to Reverse Phase Liquid Chromatography (RP-LC) of proteins or peptides, offline Liquid Chromatography (LC) prior to MALDI, 1 dimensional gel separation, 2-dimensional gel separation, Strong Cation Exchange (SCX) chromatography, Strong Anion Exchange (SAX) chromatography, Weak Cation Exchange (WCX), and Weak Anion Exchange (WAX). One or more of the above techniques can be used prior to mass spectrometric analysis.

In one aspect of the disclosure the biomarker can be detected in a biological sample using a microarray. Differential gene expression can also be identified, or confirmed using the microarray technique. Thus, the expression profile biomarkers can be measured in either fresh or fixed tissue, using microarray technology. In this method, polynucleotide sequences of interest (including cDNAs and oligonucleotides) are plated, or arrayed, on a microchip substrate. The arrayed sequences are then hybridized with specific DNA probes from cells or tissues of interest. The source of mRNA typically is total RNA isolated from a biological sample, and corresponding normal tissues or cell lines may be used to determine differential expression.

In a specific embodiment of the microarray technique, PCR amplified inserts of cDNA clones are applied to a substrate in a dense array. Preferably at least 10,000 nucleotide sequences are applied to the substrate. The microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the microarray chip is scanned by a device such as, confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pair-wise to the array. The relative abundance of the transcripts from the two sources corresponding to each specified gene is thus determined simultaneously. Microarray analysis can be performed by commercially available equipment, following manufacturer's protocols.

In one aspect of the disclosure the biomarker can be detected in a biological sample using qRT-PCR, which can be used to compare mRNA levels in different sample populations, in normal and tumor tissues, with or without drug treatment, to characterize patterns of gene expression, to discriminate between closely related mRNAs, and to analyze RNA structure. The first step in gene expression profiling by RT-PCR is extracting RNA from a biological sample followed by the reverse transcription of the RNA template into cDNA and amplification by a PCR reaction. The reverse transcription reaction step is generally primed using specific primers, random hexamers, or oligo-dT primers, depending on the goal of expression profiling. The two commonly used reverse transcriptases are avilo myeloblastosis virus reverse transcriptase (AMV-RT) and Moloney murine leukemia virus reverse transcriptase (MLV-RT).

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TaqMan™ PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan™ RT-PCR can be performed using commercially available equipment, such as, for example, ABI PRISM 7700 Sequence Detection System™ (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700™ Sequence Detection System™. The system consists of a thermocycler, laser, charge-coupled device (CCD), camera and computer. The system includes software for running the instrument and for analyzing the data. 5′-Nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and Beta-Actin.

A more recent variation of the RT-PCR technique is the real time quantitative PCR, which measures PCR product accumulation through a dual-labeled fluorigenic probe (i.e., TaqMan™ probe). Real time PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g. Held et al., Genome Research 6:986-994 (1996).

G. Data Handling

The values from the assays described above can be calculated and stored manually. Alternatively, the above-described steps can be completely or partially performed by a computer program product. The present disclosure thus provides a computer program product including a computer readable storage medium having a computer program stored on it. The program can, when read by a computer, execute relevant calculations based on values obtained from analysis of one or more biological samples from an individual (e.g., gene or protein expression levels, normalization, standardization, thresholding, and conversion of values from assays to a clinical outcome score and/or text or graphical depiction of clinical status or stage and related information). The computer program product has stored therein a computer program for performing the calculation.

The present disclosure provides systems for executing the data collection and handling or calculating software programs described above, which system generally includes: a) a central computing environment; b) an input device, operatively connected to the computing environment, to receive patient data, wherein the patient data can include, for example, gene or protein expression level or other value obtained from an assay using a biological sample from the patient, or mass spec data or data for any of the assays provided by the present disclosure; c) an output device, connected to the computing environment, to provide information to a user (e.g., medical personnel); and d) an algorithm executed by the central computing environment (e.g., a processor), where the algorithm is executed based on the data received by the input device, and wherein the algorithm calculates an expression score, thresholding, or other functions described herein. The methods provided by the present disclosure may also be automated in whole or in part.

H. Subjects

Biological samples are collected from subjects who want to determine their likelihood of having a colon tumor or polyp. The disclosure provides for subjects that can be healthy and asymptomatic. In various embodiments, the subjects are healthy, asymptomatic and between the ages 20-50. In various embodiments, the subjects are healthy and asymptomatic and have no family history of adenoma or polyps. In various embodiments, the subjects are healthy and asymptomatic and never received a colonoscopy. The disclosure also provides for healthy subjects who are having a test as part of a routine examination, or to establish baseline levels of the biomarkers.

The disclosure provides for subjects that have no symptoms for colorectal carcinoma, no family history for colorectal carcinoma, and no recognized risk factors for colorectal carcinoma. The disclosure provides for subjects that have no symptoms for colorectal carcinoma, no family history for colorectal carcinoma, and no recognized risk factors for colorectal carcinoma other than age.

Biological samples may also be collected from subjects who have been determined to have a high risk of colorectal polyps or cancer based on their family history, a who have had previous treatment for colorectal polyps or cancer and or are in remission. Biological samples may also be collected from subjects who present with physical symptoms known to be associated with colorectal cancer, subjects identified through screening assays (e.g., fecal occult blood testing or sigmoidoscopy) or rectal digital exam or rigid or flexible colonoscopy or CT scan or other x-ray techniques. Biological samples may also be collected from subjects currently undergoing treatment to determine the effectiveness of therapy or treatment they are receiving.

I. Biological Samples

The biomarkers can be measured in different types of biological samples. The sample is preferably from a biological sample that collects and surveys the entire system. Examples of a biological sample types useful in this disclosure include one or more, but are not limited to: urine, stool, tears, whole blood, serum, plasma, blood constituent, bone marrow, tissue, cells, organs, saliva, cheek swab, lymph fluid, cerebrospinal fluid, lesion exudates and other fluids produced by the body. The biomarkers can also be extracted from a biopsy sample, frozen, fixed, paraffin embedded, or fresh.

### IV. Biomarkers and Biomarker Profiles

The biomarkers of the present disclosure allow for differentiation between a healthy individual and one suffering from or at risk for the development of colon polyps and different states of colon polyps (e.g. hyperplasic, malignant, carcinoma or tumor subtype). Specifically, the present disclosure's discovery of the biomarkers provide for the diagnostic methods, kits that aid the clinical evaluation and management of colon polyps and colon cancer.

Biomarkers which can be useful for the clinical evaluation and management of colon polyps include the full proteins, peptide fragments, nucleic acids, or transitional ions of the following proteins (UNIprotein ID numbers): SPB6_HUMAN, FRIL_HUMAN, P53_HUMAN, 1A68_HUMAN, ENOA_HUMAN, TKT_HUMAN, and combinations thereof.

Biomarkers which can be useful for the clinical evaluation and management of colon polyps include the full proteins, peptide fragments, nucleic acids, or transitional ions of the following proteins (UNIprotein ID numbers): SPB6_HUMAN, FRIL_HUMAN, P53_HUMAN, 1A68_HUMAN, ENOA_HUMAN, TKT_HUMAN, TSG6_HUMAN, TPM2_HUMAN, ADT2_HUMAN, FHL1_HUMAN, CCR5_HUMAN, CEAM5_HUMAN, SPON2_HUMAN, 1A68_HUMAN, RBX1_HUMAN, COR1C_HUMAN, VIME_HUMAN, PSME3_HUMAN, and combinations thereof.

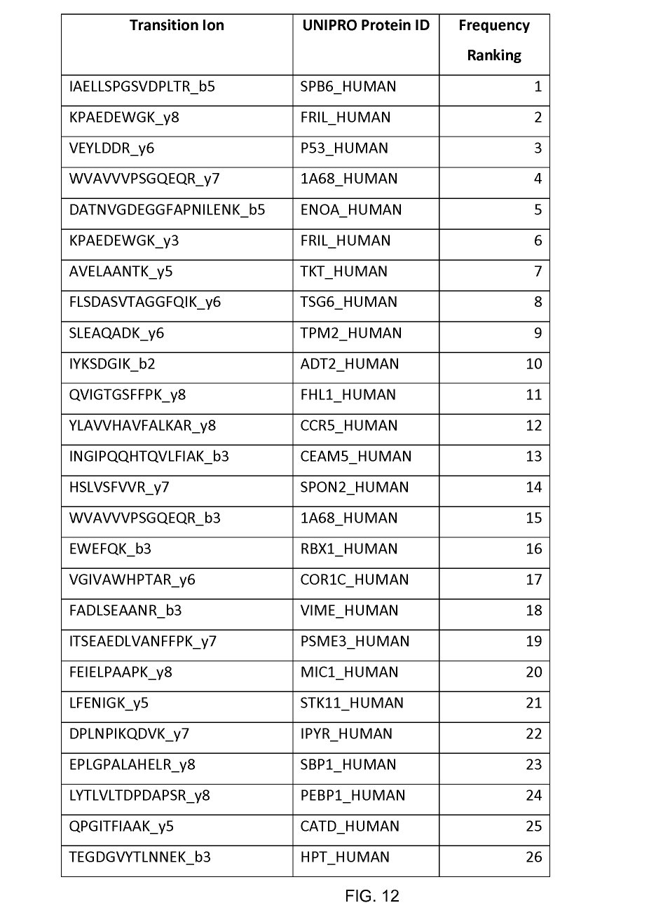

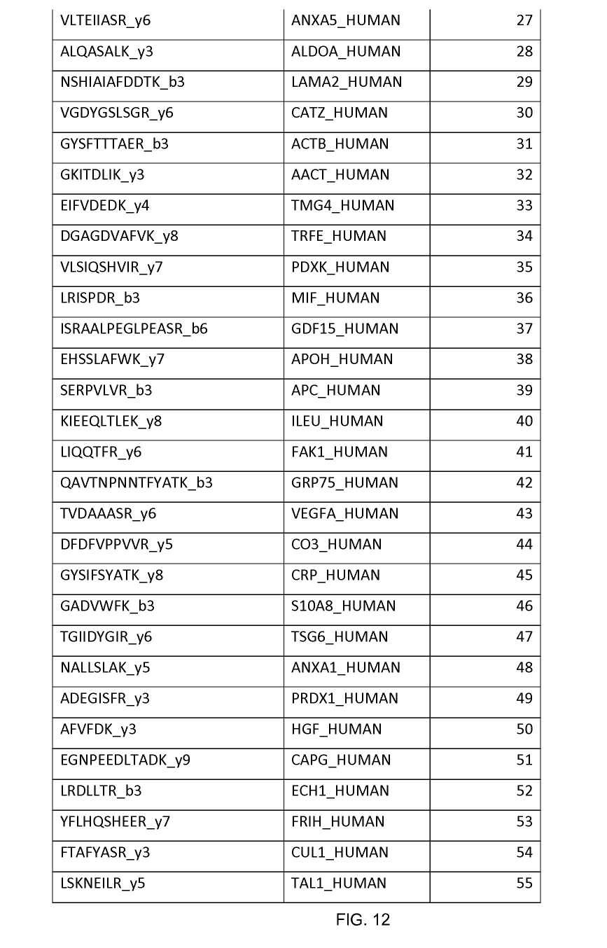

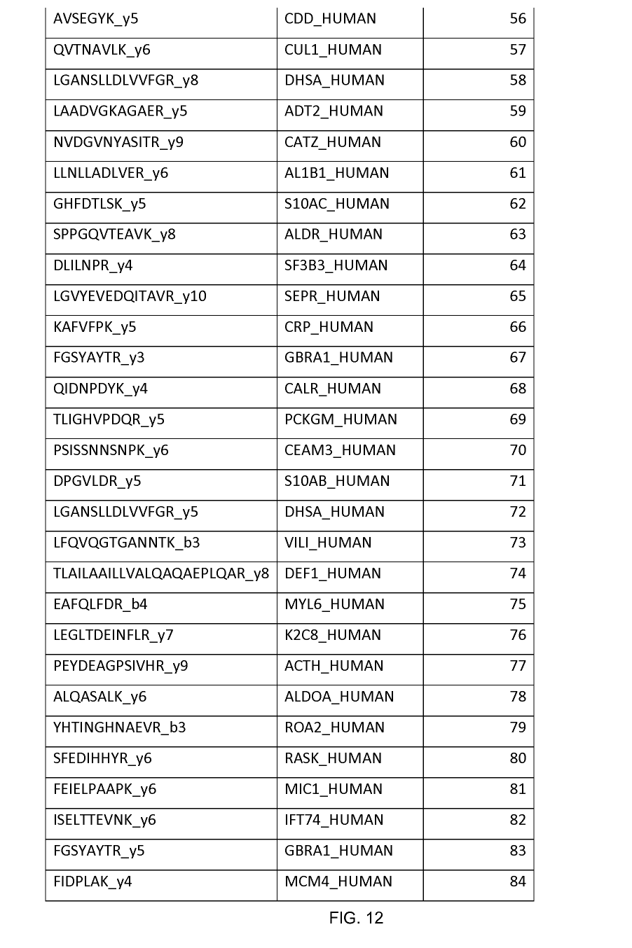

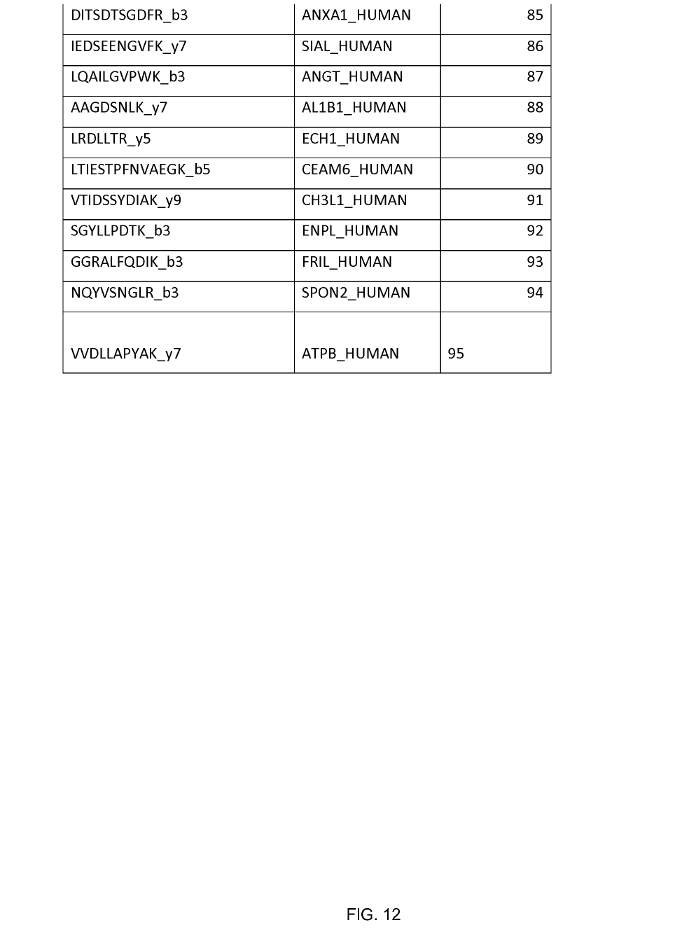

Biomarkers which can be useful for the clinical evaluation and management of colon polyps include the full proteins, peptide fragments, nucleic acids, or transitional ions of the following proteins (UNIprotein ID numbers): SPB6_HUMAN, FRIL_HUMAN, P53_HUMAN, 1A68_HUMAN, ENOA_HUMAN and TKT_HUMAN, TSG6_HUMAN, TPM2_HUMAN, ADT2_HUMAN, FHL1_HUMAN, CCR5_HUMAN, CEAM5_HUMAN, SPON2_HUMAN, 1A68_HUMAN, RBX1_HUMAN, COR1C_HUMAN, VIME_HUMAN, PSME3_HUMAN, MIC1_HUMAN, STK11_HUMAN, IPYR_HUMAN, SBP1_HUMAN, PEBP1_HUMAN, CATD_HUMAN, HPT_HUMAN, ANXA5_HUMAN, ALDOA_HUMAN, LAMA2_HUMAN, CATZ_HUMAN, ACTB_HUMAN, AACT_HUMAN, and combinations thereof Biomarkers which can be useful for the clinical evaluation and management of colon polyps include the transitional ions of FIG. 12.

The biomarker identified from whole serum by the methods of the disclosure includes full proteins, peptide fragments, nucleic acids, or transitional ions corresponding to the following proteins (UNIprotein ID numbers): Actin, cytoplasmic 1 (ACTB_HUMAN) (SEQ ID NO: 1), Actin, gamma-enteric smooth muscle precursor (ACTH_HUMAN) (SEQ ID NO: 2), Angiotensinogen precursor (ANGT_HUMAN) (SEQ ID NO: 3), Adenosylhomocysteinase (SAHH_HUMAN) (SEQ ID NO: 4), Aldose reductase (ALDR_HUMAN) (SEQ ID NO: 5), RAC-alpha serine/threonine-protein kinase (AKT1_HUMAN) (SEQ ID NO: 6), Serum albumin precursor (ALBU_HUMAN) (SEQ ID NO: 7), Retinal dehydrogenase 1 (AL1A1_HUMAN) (SEQ ID NO: 8), Aldehyde dehydrogenase X, mitochondrial precursor (AL1B1_HUMAN) (SEQ ID NO: 9), Fructose-bisphosphate aldolase A (ALDOA_HUMAN) (SEQ ID NO: 10), Alpha-amylase 2B precursor (AMY2B_HUMAN) (SEQ ID NO: 11), Annexin A1 (ANXA1_HUMAN) (SEQ ID NO: 12), Annexin A3 (ANXA3_HUMAN) (SEQ ID NO: 13), Annexin A4 (ANXA4_HUMAN) (SEQ ID NO: 14), Annexin A5 (ANXA5_HUMAN) (SEQ ID NO: 15), Adenomatous polyposis coli protein (APC_HUMAN) (SEQ ID NO: 16), Apolipoprotein A-I precursor (APOA1_HUMAN) (SEQ ID NO: 17), Apolipoprotein C-I precursor (APOC1_HUMAN) (SEQ ID NO: 18), Beta-2-glycoprotein 1 precursor (APOH HUMAN) (SEQ ID NO: 19), Rho GDP-dissociation inhibitor 1 (GDIR1_HUMAN) (SEQ ID NO: 20), ATP synthase subunit beta, mitochondrial precursor (ATPB_HUMAN) (SEQ ID NO: 21), B-cell scaffold protein with ankyrin repeats (BANK1_HUMAN) (SEQ ID NO: 22), Uncharacterized protein C18orf8 (MIC1_HUMAN) (SEQ ID NO: 23), Putative uncharacterized protein C1orf195 (CA195_HUMAN) (SEQ ID NO: 24), Complement C3 precursor (CO3_HUMAN) (SEQ ID NO: 25), Complement component C9 precursor (CO9_HUMAN) (SEQ ID NO: 26), Carbonic anhydrase 1 (CAH1_HUMAN) (SEQ ID NO: 27), Carbonic anhydrase 2 (CAH2_HUMAN) (SEQ ID NO: 28), Calreticulin precursor (CALR_HUMAN) (SEQ ID NO: 29), Macrophage-capping protein (CAPG_HUMAN) (SEQ ID NO: 30), Signal transducer CD24 precursor (CD24_HUMAN) (SEQ ID NO: 31), CD63 antigen (CD63_HUMAN) (SEQ ID NO: 32), Cytidine deaminase (CDD_HUMAN) (SEQ ID NO: 33), Carcinoembryonic antigen-related cell adhesion molecule 3 (CEAM3_HUMAN) (SEQ ID NO: 34), Carcinoembryonic antigen-related cell adhesion molecule 5 (CEAM5_HUMAN) (SEQ ID NO: 35), Carcinoembryonic antigen-related cell adhesion molecule 6 (CEAM6_HUMAN) (SEQ ID NO: 36), Choriogonadotropin subunit beta precursor (CGHB_HUMAN) (SEQ ID NO: 37), Chitinase-3-like protein 1 precursor (CH3L1_HUMAN) (SEQ ID NO: 38), Creatine kinase B-type (KCRB_HUMAN) (SEQ ID NO: 39), C-type lectin domain family 4 member D (CLC4D_HUMAN) (SEQ ID NO: 40), Clusterin precursor (CLUS_HUMAN) (SEQ ID NO: 41), Calponin-1 (CNN1_HUMAN) (SEQ ID NO: 42), Coronin-1C(COR1C_HUMAN) (SEQ ID NO: 43), C-reactive protein precursor (CRP HUMAN) (SEQ ID NO: 44), Macrophage colony-stimulating factor 1 precursor (CSF1_HUMAN) (SEQ ID NO: 45), Catenin beta-1 (CTNB1_HUMAN) (SEQ ID NO: 46), Cathepsin D precursor (CATD_HUMAN) (SEQ ID NO: 47), Cathepsin S precursor (CATS_HUMAN) (SEQ ID NO: 48), Cathepsin Z precursor (CATZ_HUMAN) (SEQ ID NO: 49), Cullin-1 (CUL1_HUMAN) (SEQ ID NO: 50), Aspartate-tRNA ligase, cytoplasmic (SYDC_HUMAN) (SEQ ID NO: 51), Neutrophil defensin 1 (DEF1_HUMAN) (SEQ ID NO: 52), Neutrophil defensin 3 (DEF3_HUMAN) (SEQ ID NO: 53), Desmin (DESM HUMAN) (SEQ ID NO: 54), Dipeptidyl peptidase 4 (DPP4_HUMAN) (SEQ ID NO: 55), Dihydropyrimidinase-related protein 2 (DPYL2_HUMAN) (SEQ ID NO: 56), Cytoplasmic dynein 1 heavy chain 1 (DYHC1_HUMAN) (SEQ ID NO: 57), Delta(3,5)-Delta(2,4)-dienoyl-CoA isomerase, mitochondrial precursor (ECH1_HUMAN) (SEQ ID NO: 58), Elongation factor 2 (EF2_HUMAN) (SEQ ID NO: 59), Eukaryotic initiation factor 4A-III (IF4A3_HUMAN) (SEQ ID NO: 60), Alpha-enolase (ENOA_HUMAN) (SEQ ID NO: 61), Ezrin (EZRI_HUMAN) (SEQ ID NO: 62), Niban-like protein 2 (NIBL2_HUMAN) (SEQ ID NO: 63), Seprase (SEPR_HUMAN) (SEQ ID NO: 64), F-box only protein 4 (FBX4_HUMAN) (SEQ ID NO: 65), Fibrinogen beta chain precursor (FIBB_HUMAN) (SEQ ID NO: 66), Fibrinogen gamma chain (FIBG HUMAN) (SEQ ID NO: 67), Four and a half LIM domains protein 1 (FHL1_HUMAN) (SEQ ID NO: 68), Filamin-A (FLNA_HUMAN) (SEQ ID NO: 69), FERM domain-containing protein 3 (FRMD3_HUMAN) (SEQ ID NO: 70), Ferritin heavy chain (FRIH HUMAN) (SEQ ID NO: 71), Ferritin light chain (FRIL_HUMAN) (SEQ ID NO: 72), Tissue alpha-L-fucosidase precursor (FUCO_HUMAN) (SEQ ID NO: 73), Gamma-aminobutyric acid receptor subunit alpha-1 precursor (GBRA1_HUMAN) (SEQ ID NO: 74), Glyceraldehyde-3-phosphate dehydrogenase (G3P HUMAN) (SEQ ID NO: 75), Glycine-tRNA ligase (SYG HUMAN) (SEQ ID NO: 76), Growth/differentiation factor 15 precursor (GDF15_HUMAN) (SEQ ID NO: 77), Gelsolin precursor (GELS_HUMAN) (SEQ ID NO: 78), Glutathione S-transferase P (GSTP1_HUMAN) (SEQ ID NO: 79), Hyaluronan-binding protein 2 precursor (HABP2_HUMAN) (SEQ ID NO: 80), Hepatocyte growth factor precursor (HGF HUMAN) (SEQ ID NO: 81), HLA class I histocompatibility antigen, A-68 alpha chain (1A68_HUMAN) (SEQ ID NO: 82), High mobility group protein B1 (HMGB1_HUMAN) (SEQ ID NO: 83), Heterogeneous nuclear ribonucleoprotein A1 (ROA1_HUMAN) (SEQ ID NO: 84), Heterogeneous nuclear ribonucleoproteins A2/B1 (ROA2_HUMAN) (SEQ ID NO: 85), Heterogeneous nuclear ribonucleoprotein F (HNRPF_HUMAN) (SEQ ID NO: 86), Haptoglobin precursor (HPT_HUMAN) (SEQ ID NO: 87), Heat shock protein HSP 90-beta (HS90B_HUMAN) (SEQ ID NO: 88), Endoplasmin precursor (ENPL_HUMAN) (SEQ ID NO: 89), Stress-70 protein, mitochondrial precursor (GRP75_HUMAN) (SEQ ID NO: 90), Heat shock protein beta-1 (HSPB1_HUMAN) (SEQ ID NO: 91), 60 kDa heat shock protein, mitochondrial (CH60_HUMAN) (SEQ ID NO: 92), Bone sialoprotein 2 (SIAL_HUMAN) (SEQ ID NO: 93), Intraflagellar transport protein 74 homolog (IFT74_HUMAN) (SEQ ID NO: 94), Insulin-like growth factor I (IGF1_HUMAN) (SEQ ID NO: 95), Ig alpha-2 chain C region (IGHA2_HUMAN) (SEQ ID NO: 96), Interleukin-2 receptor subunit beta precursor (IL2RB_HUMAN) (SEQ ID NO: 97), Interleukin-8 (IL8_HUMAN) (SEQ ID NO: 98), Interleukin-9 (IL9_HUMAN) (SEQ ID NO: 99), GTPase KRas precursor (RASK_HUMAN) (SEQ ID NO: 100), Keratin, type I cytoskeletal 19 (K1C19_HUMAN) (SEQ ID NO: 101), Keratin, type II cytoskeletal 8 (K2C8_HUMAN) (SEQ ID NO: 102), Laminin subunit alpha-2 precursor (LAMA2_HUMAN) (SEQ ID NO: 103), Galectin-3 (LEG3_HUMAN) (SEQ ID NO: 104), Lamin-B1 precursor (LMNB1_HUMAN) (SEQ ID NO: 105), Microtubule-associated protein RP/EB family member 1 (MARE1_HUMAN) (SEQ ID NO: 106), DNA replication licensing factor MCM4 (MCM4_HUMAN) (SEQ ID NO: 107), Macrophage migration inhibitory factor (MIF_HUMAN) (SEQ ID NO: 108), Matrilysin precursor (MMP7_HUMAN) (SEQ ID NO: 109), Matrix metalloproteinase-9 precursor (MMP9_HUMAN) (SEQ ID NO: 110), B-lymphocyte antigen CD20 (CD20_HUMAN) (SEQ ID NO: 111), Myosin light polypeptide 6 (MYL6_HUMAN) (SEQ ID NO: 112), Myosin regulatory light polypeptide 9 (MYL9_HUMAN) (SEQ ID NO: 113), Nucleoside diphosphate kinase A (NDKA_HUMAN) (SEQ ID NO: 114), Nicotinamide N-methyltransferase (NNMT_HUMAN) (SEQ ID NO: 115), Alpha-1-acid glycoprotein 1 precursor (A1AG1_HUMAN) (SEQ ID NO: 116), Phosphoenolpyruvate carboxykinase [GTP], mitochondrial precursor (PCKGM HUMAN) (SEQ ID NO: 117), Protein disulfide-isomerase A3 precursor (PDIA3_HUMAN) (SEQ ID NO: 118), Protein disulfide-isomerase A6 precursor (PDIA6_HUMAN) (SEQ ID NO: 119), Pyridoxal kinase (PDXK_HUMAN) (SEQ ID NO: 120), Phosphatidylethanolamine-binding protein 1 (PEBP1_HUMAN) (SEQ ID NO: 121), Phosphatidylinositol transfer protein alpha isoform (PIPNA_HUMAN) (SEQ ID NO: 122), Pyruvate kinase isozymes M1/M2 (KPYM HUMAN) (SEQ ID NO: 123), Urokinase-type plasminogen activator precursor (UROK_HUMAN) (SEQ ID NO: 124), Inorganic pyrophosphatase (IPYR_HUMAN) (SEQ ID NO: 125), Peroxiredoxin-1 (PRDX1_HUMAN) (SEQ ID NO: 126), Serine/threonine-protein kinase D1 (KPCD1_HUMAN) (SEQ ID NO: 127), Prolactin (PRL_HUMAN) (SEQ ID NO: 128), Transmembrane gamma-carboxyglutamic acid protein 4 precursor (TMG4_HUMAN) (SEQ ID NO: 129), Proteasome activator complex subunit 3 (PSME3_HUMAN) (SEQ ID NO: 130), Phosphatidylinositol 3,4,5-trisphosphate 3-phosphatase and dual-specificity protein phosphatase PTEN (PTEN_HUMAN) (SEQ ID NO: 131), Focal adhesion kinase 1 (FAK1_HUMAN) (SEQ ID NO: 132), Protein-tyrosine kinase 2-beta (FAK2_HUMAN) (SEQ ID NO: 133), E3 ubiquitin-protein ligase RBX1 (RBX1_HUMAN) (SEQ ID NO: 134), Regenerating islet-derived protein 4 precursor (REG4_HUMAN) (SEQ ID NO: 135), Transforming protein RhoA (RHOA_HUMAN) (SEQ ID NO: 136), Rho-related GTP-binding protein RhoB (RHOB_HUMAN) (SEQ ID NO: 137), Rho-related GTP-binding protein RhoC (RHOC_HUMAN) (SEQ ID NO: 138), 40S ribosomal protein SA (RSSA_HUMAN) (SEQ ID NO: 139), Ribosome-binding protein 1 (RRBP1_HUMAN) (SEQ ID NO: 140), Protein S100-All (S10AB_HUMAN) (SEQ ID NO: 141), Protein S100-A12 (S10AC_HUMAN) (SEQ ID NO: 142), Protein S100-A8 (S10A8_HUMAN) (SEQ ID NO: 143), Protein S100-A9 (S10A9_HUMAN) (SEQ ID NO: 144), Serum amyloid A-1 protein (SAM HUMAN) (SEQ ID NO: 145), Serum amyloid A-2 protein precursor (SAA2_HUMAN) (SEQ ID NO: 146), Secretagogin (SEGN_HUMAN) (SEQ ID NO: 147), Serologically defined colon cancer antigen 3 (SDCG3_HUMAN) (SEQ ID NO: 148), Succinate dehydrogenase [ubiquinone] flavoprotein subunit, mitochondrial precursor (DHSA_HUMAN) (SEQ ID NO: 149), Selenium-binding protein 1 (SBP1_HUMAN) (SEQ ID NO: 150), P-selectin glycoprotein ligand 1 precursor (SELPL_HUMAN) (SEQ ID NO: 151), Septin-9 (SEPT9_HUMAN) (SEQ ID NO: 152), Alpha-1-antitrypsin precursor (A1AT_HUMAN) (SEQ ID NO: 153), Alpha-1-antichymotrypsin precursor (AACT_HUMAN) (SEQ ID NO: 154), Leukocyte elastase inhibitor (ILEU HUMAN) (SEQ ID NO: 155), Serpin B6 (SPB6_HUMAN) (SEQ ID NO: 156), Splicing factor 3B subunit 3 (SF3B3_HUMAN) (SEQ ID NO: 157), S-phase kinase-associated protein 1 (SKP1_HUMAN) (SEQ ID NO: 158), ADP/ATP translocase 2 (ADT2_HUMAN) (SEQ ID NO: 159), Pancreatic secretory trypsin inhibitor (ISK1_HUMAN) (SEQ ID NO: 160), Spondin-2 (SPON2_HUMAN) (SEQ ID NO: 161), Osteopontin (OSTP HUMAN) (SEQ ID NO: 162), Proto-oncogene tyrosine-protein kinase Src (SRC_HUMAN) (SEQ ID NO: 163), Serine/threonine-protein kinase STK11 (STK11_HUMAN) (SEQ ID NO: 164), Heterogeneous nuclear ribonucleoprotein Q (HNRPQ_HUMAN) (SEQ ID NO: 165), T-cell acute lymphocytic leukemia protein 1 (TAL1_HUMAN) (SEQ ID NO: 166), Serotransferrin precursor (TRFE_HUMAN) (SEQ ID NO: 167), Thrombospondin-1 precursor (TSP1_HUMAN) (SEQ ID NO: 168), Metalloproteinase inhibitor 1 (TIMP1_HUMAN) (SEQ ID NO: 169), Transketolase (TKT_HUMAN) (SEQ ID NO: 170), Tumor necrosis factor-inducible gene 6 protein precursor (TSG6_HUMAN) (SEQ ID NO: 171), Tumor necrosis factor receptor superfamily member 10B (TR10B_HUMAN) (SEQ ID NO: 172), Tumor necrosis factor receptor superfamily member 6B (TNF6B_HUMAN) (SEQ ID NO: 173), Cellular tumor antigen p53 (P53_HUMAN) (SEQ ID NO: 174), Tropomyosin beta chain (TPM2_HUMAN) (SEQ ID NO: 175), Translationally-controlled tumor protein (TCTP_HUMAN) (SEQ ID NO: 176), Heat shock protein 75 kDa, mitochondrial precursor (TRAP1_HUMAN) (SEQ ID NO: 177), Thiosulfate sulfurtransferase (THTR_HUMAN) (SEQ ID NO: 178), Tubulin beta-1 chain (TBB1_HUMAN) (SEQ ID NO: 179), UDP-glucose 6-dehydrogenase (UGDH_HUMAN) (SEQ ID NO: 180), UTP-glucose-1-phosphate uridylyltransferase (UGPA_HUMAN) (SEQ ID NO: 181), Vascular endothelial growth factor A (VEGFA_HUMAN) (SEQ ID NO: 182), Villin-1 (VILI_HUMAN) (SEQ ID NO: 183), Vimentin (VIME_HUMAN) (SEQ ID NO: 184), Pantetheinase precursor (VNN1_HUMAN) (SEQ ID NO: 185), 14-3-3 protein zeta/delta (1433Z_HUMAN) (SEQ ID NO: 186), C-C chemokine receptor type 5 (CCR5_HUMAN) (SEQ ID NO: 187), or Plasma alpha-L-fucosidase (FUCO2_HUMAN) (SEQ ID NO: 188). The methods of the present invention contemplate determining the expression level of at least one, at least two, at least three, at least four, at least five, at least six, at least seven, at least eight, at least nine biomarkers provide above. The methods may involve determination of the expression levels of at least ten, at least fifteen, or at least twenty of the biomarkers provide above.

For all aspects of the present disclosure, the methods may further include determining the expression level of at least two biomarkers provide herein. It is further contemplated that the methods of the present disclosure may further include determining the expression levels of at least three, at least four, at least five, at least six, at least seven, at least eight, at least nine biomarkers provide herein. The methods may involve determination of the expression levels of at least ten, at least fifteen, or at least twenty of the biomarkers provide herein.

The biomarker identified from whole serum by the methods of the disclosure includes peptide/protein fragments or genes corresponding to the following proteins: SCDC26 (CD26), CEA molecule 5 (CEACAM5), CA195 (CCR5), CA19-9, M2PK (PKM2), TIMP1, P-selectin (SELPLG), VEGFA, HcGB (CGB), VILLIN, TATI (SPINK1), and A-L-fucosidase (FUCA2). Groupings of two, three, four, five, six, seven, eight, nine, ten, eleven, and all twelve of the above proteins or genes are included. Such groupings may exclude proteins or genes within this set or may exclude additional proteins or genes, or may further comprise additional proteins.

The biomarker identified from whole serum by the methods of the disclosure includes peptide/protein fragments or genes corresponding to the following proteins: ANXA5, GAPDH, PKM2, ANXA4, GARS, RRBP1, KRT8, SYNCRIP, S100A9, ANXA3, CAPG, HNRNPF, PPA1, NME1, PSME3, AHCY, TPT1, HSPB1, and RPSA. Groupings of two, three, four, five, six, seven, eight, nine, ten, eleven, twelve, thirteen, fourteen, fifteen, sixteen, seventeen, eighteen, and all nineteen of the above proteins or genes are included. Such groupings may exclude proteins or genes within this set or may exclude additional proteins or genes, or may further comprise additional proteins.

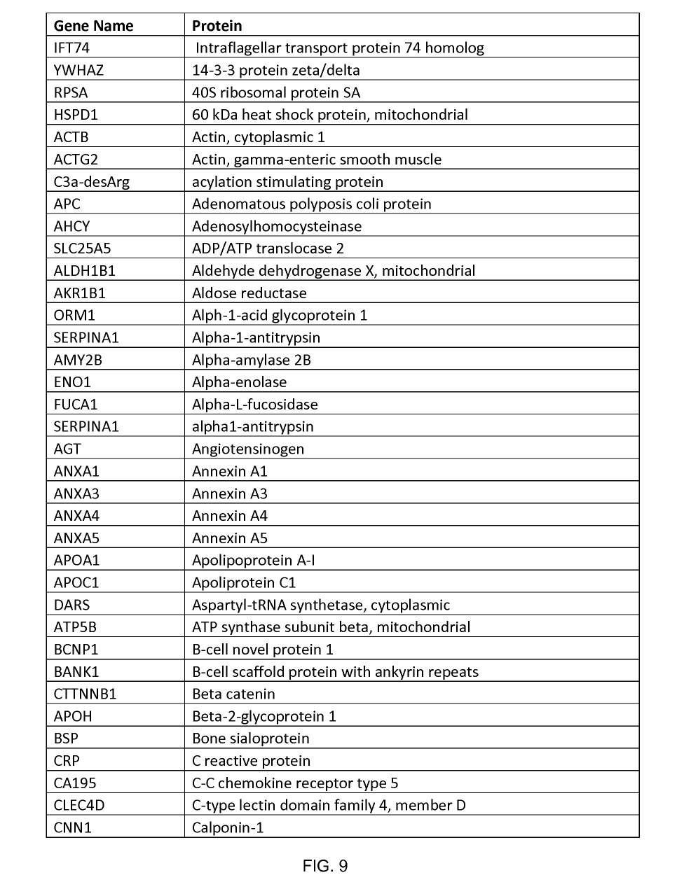

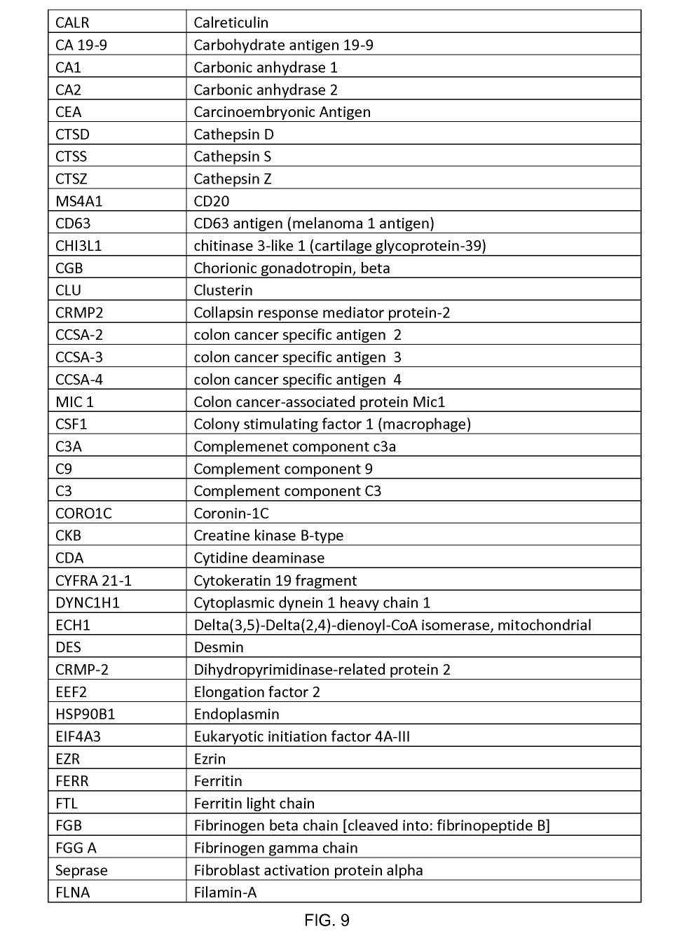

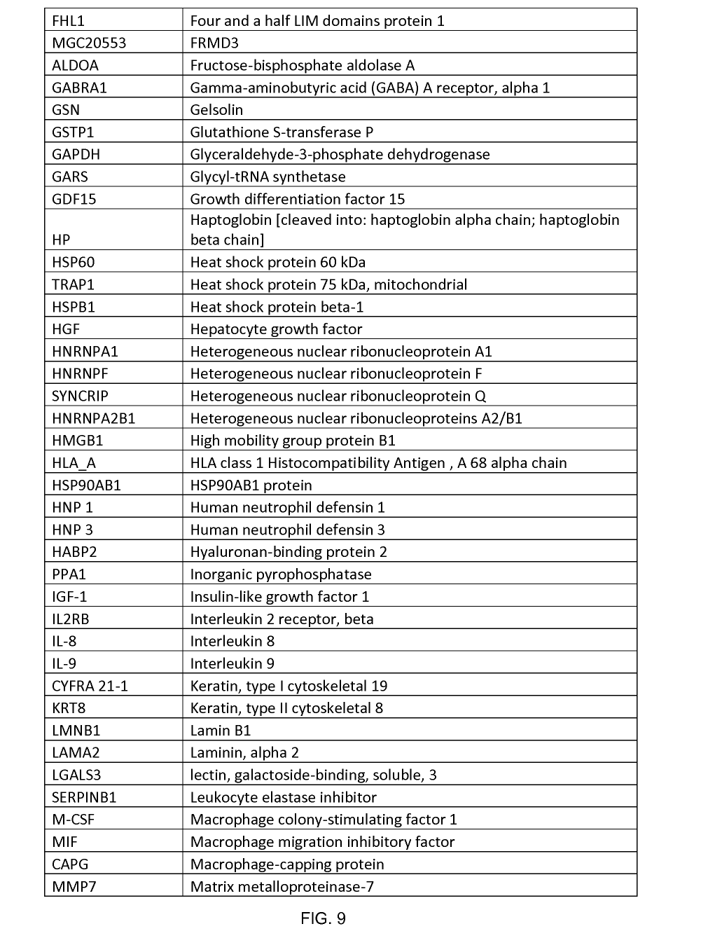

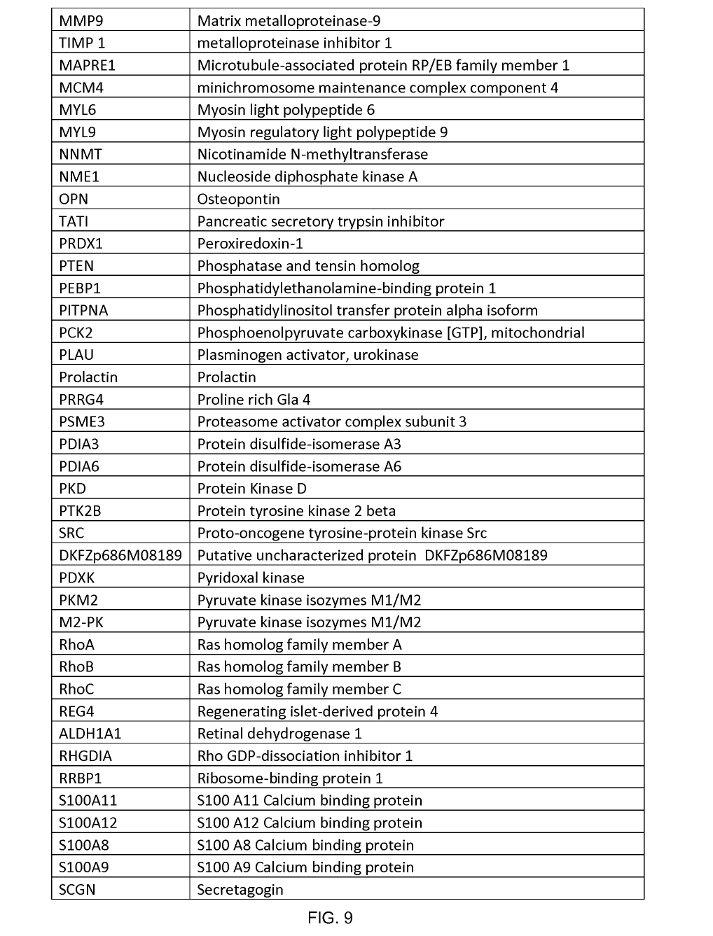

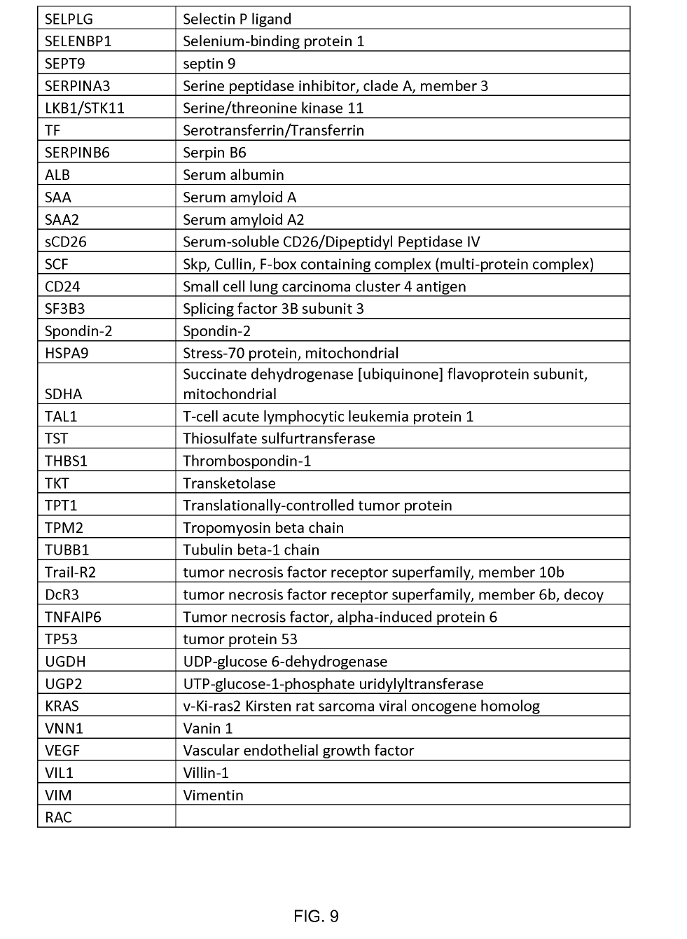

The biomarker identified from whole serum by the methods of the disclosure includes peptide/protein fragments or genes corresponding to the proteins identified in FIG. 9. Groupings of two, three, four, five, six, seven, eight, nine, ten, eleven, twelve, and more of the above proteins or genes are included. Such groupings may exclude proteins or genes within this set or may exclude additional proteins, or may further comprise additional proteins.

It is known that proteins frequently exist in a sample in a plurality of different forms as they can associate in various forms for various protein complexes. These forms can result from either, or both, of pre- and post-translational modification. Pre-translational modified forms include allelic variants, slice variants and RNA editing forms. In such instances, it is know that gene expression product will present in various homologies to proteins defined in the human databases. Therefore the disclosure appreciates that there can be various versions of the defined biomarkers. For instance, said sequence homology is selected from the group of greater than 75%, greater than 80%, greater than 85%, greater than 90%, greater than 95%, and greater than 99%. Additionally, there can be post-translationally modified forms of the biomarkers. Post-translationally modified forms include, but are not limited to, forms resulting from proteolytic cleavage (e.g., fragments of a parent protein), glycosylation, phosphorylation, lipidation, oxidation, methylation, cystinylation, sulphonation and acetylation of the protein biomarkers.

The biomarkers of the present disclosure include the full-length protein, their corresponding RNA or DNA and all modified forms. Modified forms of the biomarker include for example any splice-variants of the disclosed biomarkers and their corresponding RNA or DNA which encode them. In certain cases the modified forms, or truncated versions of the proteins, or their corresponding RNA or DNA, may exhibit better discriminatory power in diagnosis than the full-length protein.

A truncated or fragment of a protein, polypeptide or peptide generally refers to N-terminally and/or C-terminally deleted or truncated forms of said protein, polypeptide or peptide. The term encompasses fragments arising by any mechanism, such as, without limitation, by alternative translation, exo- and/or endo-proteolysis and/or degradation of said peptide, polypeptide or protein, such as, for example, in vivo or in vitro, such as, for example, by physical, chemical and/or enzymatic proteolysis. Without limitation, a truncated or fragment of a protein, polypeptide or peptide may represent at least about 5%, or at least about 10%, e.g., >20%, >30% or >40%, such as >50%, e.g., >60%, >70%, or >80%, or even 90% or >95% of the amino acid sequence of said protein, polypeptide or peptide.

Without limitation, a truncated or fragment of a protein may include a sequence of 5 consecutive amino acids, or 10 consecutive amino acids, or 20 consecutive amino acids, or 30 consecutive amino acids, or more than 50 consecutive amino acids, e.g., 60, 70, 80, 90, 100, 200, 300, 400, 500 or 600 consecutive amino acids of the corresponding full length protein.

In some instances, a fragment may be N-terminally and/or C-terminally truncated by between 1 and about 20 amino acids, such as, e.g., by between 1 and about 15 amino acids, or by between 1 and about 10 amino acids, or by between 1 and about 5 amino acids, compared to the corresponding mature, full-length protein or its soluble or plasma circulating form.

Any protein biomarker of the present disclosure such as a peptide, polypeptide or protein and fragments thereof may also encompass modified forms of said marker, peptide, polypeptide or protein and fragments such as bearing post-expression modifications including but not limited to, modifications such as phosphorylation, glycosylation, lipidation, methylation, cysteinylation, sulphonation, glutathionylation, acetylation, oxidation of methionine to methionine sulphoxide or methionine sulphone, and the like.

In some instances, fragments of a given protein, polypeptide or peptide may be achieved by in vitro proteolysis of said protein, polypeptide or peptide to obtain advantageously detectable peptide(s) from a sample. For example, such proteolysis may be effected by suitable physical, chemical and/or enzymatic agents, e.g., proteinases, preferably endoproteinases, i.e., protease cleaving internally within a protein, polypeptide or peptide chain.

Suitable non-limiting examples of endoproteinases include but are not limited to serine proteinases (EC 3.4.21), threonine proteinases (EC 3.4.25), cysteine proteinases (EC 3.4.22), aspartic acid proteinases (EC 3.4.23), metalloproteinases (EC 3.4.24) and glutamic acid proteinases. Exemplary non-limiting endoproteinases include trypsin, chymotrypsin, elastase, Lysobacter enzymogenes endoproteinase Lys-C, Staphylococcus aureus endoproteinase Glu-C (endopeptidase V8) or Clostridium histolyticum endoproteinase Arg-C (clostripain).

Preferably, the proteolysis may be effected by endopeptidases of the trypsin type (EC 3.4.21.4), preferably trypsin, such as, without limitation, preparations of trypsin from bovine pancreas, human pancreas, porcine pancreas, recombinant trypsin, Lys-acetylated trypsin, trypsin in solution, trypsin immobilised to a solid support, etc. Trypsin is particularly useful, inter alia due to high specificity and efficiency of cleavage. The disclosure also provide for the use of any trypsin-like protease, i.e., with a similar specificity to that of trypsin. Otherwise, chemical reagents may be used for proteolysis. By way of example only, CNBr can cleave at Met; BNPS-skatole can cleave at Trp. The conditions for treatment, e.g., protein concentration, enzyme or chemical reagent concentration, pH, buffer, temperature, time, can be determined by the skilled person depending on the enzyme or chemical reagent employed. Further known or yet to be identified enzymes may be used with the present disclosure on the basis of their cleavage specificity and frequency to achieve desired peptide forms.

In some instances, a fragmented protein or peptide may be N-terminally and/or C-terminally truncated and is one or all transitional ions of the N-terminally (a, b, c-ion) and/or C-terminally (x, y, z-ion) truncated protein or peptide. For example, if the peptide fragment is comprised of the amino acid sequence IAELLSPGSVDPLTR then a transitional ion biomarker of the peptide fragment can include the one or more of the following transitional ion biomarkers provided in TABLE 1.

TABLE 1
Example of all transitional ions for the
peptide sequence IAELLSPGSVDPLTR

| Transitional Ion | Amino Acid Sequence |
|---|---|
| b1 | I |
| b2 | IA |
| b3 | IAE |
| b4 | IAEL |
| b5 | IAELL |
| b6 | IAELLS |
| b7 | IAELLSP |
| b8 | IAELLSPG |
| b9 | IAELLSPGS |
| b10 | IAELLSPGSV |
| b11 | IAELLSPGSVD |
| b12 | IAELLSPGSVDP |
| b13 | IAELLSPGSVDPL |
| b14 | IAELLSPGSVDPLT |
| y14 | AELLSPGSVDPLTR |
| y13 | ELLSPGSVDPLTR |
| y12 | LLSPGSVDPLTR |
| y11 | LSPGSVDPLTR |
| y10 | SPGSVDPLTR |
| y9 | PGSVDPLTR |
| y8 | GSVDPLTR |
| y7 | SVDPLTR |
| y6 | VDPLTR |
| y5 | DPLTR |
| y4 | PLTR |
| y3 | LTR |
| y2 | TR |
| y1 | R |

The biomarkers of the present disclosure include the binding partners of SCDC26 (CD26), CEA molecule 5 (CEACAM5), CA195 (CCR5), CA19-9, M2PK (PKM2), TIMP1, P-selectin (SELPLG), VEGFA, HcGB (CGB), VILLIN, TATI (SPINK1), and A-L-fucosidase (FUCA2). Groupings of two, three, four, five, six, seven, eight, nine, ten, eleven, and all twelve of the above proteins are included. Such groupings may exclude proteins within this set or may exclude additional proteins, or may further comprise additional proteins.

The biomarkers of the present disclosure include the binding partners of ANXA5, GAPDH, PKM2, ANXA4, GARS, RRBP1, KRT8, SYNCRIP, S100A9, ANXA3, CAPG, HNRNPF, PPA1, NME1, PSME3, AHCY, TPT1, HSPB1, and RPSA. Groupings of two, three, four, five, six, seven, eight, nine, ten, eleven, twelve, thirteen, fourteen, fifteen, sixteen, seventeen, eighteen, and all nineteen of the above proteins are included. Such groupings may exclude proteins within this set or may exclude additional proteins, or may further comprise additional proteins.

Exemplary human markers, nucleic acids, proteins or polypeptides as taught herein may be as annotated under NCBI Genbank (http://www.ncbi.nlm.nih.gov/) or Swissprot/Uniprot (http://www.uniprot.org/) accession numbers. In some instances said sequences may be of precursors (e.g., preproteins) of the of markers, nucleic acids, proteins or polypeptides as taught herein and may include parts which are processed away from mature molecules. In some instances although only one or more isoforms may be disclosed, all isoforms of the sequences are intended.

The biomarkers of the present disclosure include the binding partners of the proteins identified in FIG. 9. Groupings of two, three, four, five, six, seven, eight, nine, ten, eleven, twelve, and more of the above proteins are included. Such groupings may exclude proteins within this set or may exclude additional proteins, or may further comprise additional proteins.

The above-identified biomarkers are examples of biomarkers, as determined by molecular weights and partial sequences, identified by the methods of the disclosure and serve merely as an illustrative example and are not meant to limit the disclosure in any way. Suitable methods can be used to detect one or more of the biomarkers or modified biomarkers are described herein. In some aspect the disclosure provides for performing an analysis of the biological sample for the presence additional biomarkers of one or more analytes selected from the groups consisting of metabolites, DNA sequences, RNA sequences, and combinations thereof. The biomarkers listed herein can be further combined with other information such as genetic analysis, for example such as whole genome DNA or RNA sequencing from subjects.

All aspects of the present disclosure may also be practiced with a limited number of the disclosed biomarkers, their binding partners, splice-variants and corresponding DNA and RNA.

In addition to the corresponding DNA and RNA, variations found within DNA and RNA of the biomarker provide by the present disclosure may provide a means for distinguishing clinical status of an individual. Examples of such DNA and RNA genetic variation markers that can be used with the present methods include but are not limited to restriction fragment length polymorphisms, single nucleotide DNA polymorphisms, single nucleotide cDNA polymorphisms, single nucleotide RNA polymorphisms, single nucleotide RNA polymorphisms, insertions, deletions, indels, microsatellite repeats (simple sequence repeats), minisatellite repeats (variable number of tandem repeats), short tandem repeats, transposable elements, randomly amplified polymorphic DNA, and amplification fragment length polymorphism.

Biomarker Profiles

The present methods of the disclosure also provide for biomarker profiles to be generated and use in a commercial medical diagnostic product or kits.

The methods provide for biomarker profiles to be determined in a number of ways and may be the combination of measurable biomarkers or aspects of biomarkers using methods such as ratios, or other more complex association methods or algorithms (e.g., rule-based methods). A biomarker profile can comprise at least two measurements, where the measurements can correspond to the same or different biomarkers. A biomarker profile may also comprise at least 3, 4, 5, 10, 15, 20, 25, 30, 35, 40, 45, 50, 55 or more measurements. In some applications, a biomarker profile comprises hundreds, or even thousands, of measurements. A biomarker profile may comprise of measurements only from an individual, or from and individual and of measurements from a stratified population known to be related to the individual or a stratified population known not to be related to the individual, or both.

In addition, the biomarker profiles also provide for the presence or absence or quantity of the biomarkers provided herein may be evaluated each separately and independently, or the presence or absence and/or quantity of such other biomarkers may be included within subject profiles or reference profiles established in the methods disclosed herein.

### V. Applications of Biomarkers

In general the method includes at least the following steps: (a) obtaining a biological sample, (b) performing analysis of biological sample, (c) comparing the sample to a reference control, and (d) correlating the presence or amount of proteins with a subject's colon polyp status. In some aspects of the disclosure, quantification involves normalizing measurements to internal standard controls known to be at a constant level. In other aspects of the disclosure, quantification involves comparing to reference controls from healthy non-diseased subjects with no tumors and determining differential expression. In other aspects of the disclosure, quantification involves comparing to reference controls from diseased subjects with tumors and determining differential expression. Data obtained from this method can be used to create a “profile” used to predict disease state, recurrence, or response to treatment. Test results may be compared to a standard profile once it is created and correlations to responses may be derived. It should be understood the profiles described are generally optimized. The present disclosure is not limited to the use of this particular biomarker profile. Any combination of one or more markers that provides useful information can be used in the methods of the present disclosure. For example, it should be understood that one or more markers can be added or subtracted from the signatures, while maintaining the ability of the signatures to yield useful information.

In one aspect of the disclosure, quantification of all or some or a combination of the biomarkers can be used to detect the likelihood of the presence of a colon polyp in a subject. In another aspect of the disclosure, all or some or a combination of the biomarkers can be used to detect the nature of the colon tumor the identification of one or more properties of a sample in a subject, including but not limited to, the presence of benign, type of polyp, pre-cancerous stage, degree of dysplasia, subtype adenomatous polyp, or subtype of benign colon tumor disease and prognosis. In one aspect of the disclosure, all or some or a combination of the biomarkers can be used to the likelihood of developing colon tumors or polyps. In one aspect of the disclosure, all or some or a combination of the biomarkers can be used to rule out the presence of a colon tumor or polyp, i.e., to determine the absence of a colon polyp, carcinoma or both in a subject. In another aspect of the disclosure, all or some or a combination of the biomarkers can be used determined the nature of the tumor, that is whether it is a benign tumor polyp, malignant tumor, adenomatous polyp, pedunculated polyp or sessile polyp type.

In one aspect of the disclosure, all or some or a combination of the biomarkers can be used to generate a report that aids in the next steps for the clinical management of the colorectal cancer or a colon tumor. In one aspect of the disclosure, all or some or a combination of the biomarkers can be used to monitor the responsiveness to various treatments for colorectal cancer or colon tumors. In one aspect of the disclosure, all or some or a combination of the biomarkers can be used to monitor a subject that has a predisposition for developing colorectal cancer or colon tumors. In one aspect of the disclosure, all or some or a combination of the biomarkers can be used to monitor a subject for reoccurrence of colorectal cancer or colon tumors. In one aspect of the disclosure, all or some or a combination of the biomarkers can be used to monitor a subject recurrence of colorectal cancer or polyps.

In some embodiments, the method comprises identifying a profile of the biomarkers in the cells of the biological sample from a subject wherein said pattern is correlated to the likelihood of disease or condition or response.

In some aspects of this method, the one more of the biomarker or a biomarker profile is detected by quantifying expression levels of proteins by, for example, quantitative immunofluorescence or ELISA-based assay, flow cytometry or other immunoassay provide herein. In some aspects of this method the biomarker profile is detected expression levels of polynucleotides by, for example, by real-time PCR using primer sets that specifically amplify the biomarkers corresponding DNA or RNA. In another aspect of the disclosure the profile is detected by a biochip that contains capture features for biomarkers (e.g. antibodies, probes, ect.). Biochips can detect the presence of a biomarker profile by expression levels of polynucleotides, for example mRNA, in a biological sample or from a subject, alternatively, by expression levels of proteins in a patient sample using, for example, antibodies. In another some embodiment, a tumor cell profile is detected by real-time PCR using primer sets that specifically amplify the genes comprising the cancer stem cell signature. In other embodiments of the disclosure, microarrays are provided that contain polynucleotides or proteins (i.e. antibodies) that detect the expression of a cancer stem cell signature for use in prognosis.

A biological sample's biomarker profile may be compared to a reference profile and results can be determined. In one aspect of the disclosure, data generated from the tests described herein are compared to a reference profile defined by a profile model derived from measurements from one or a plurality of biological samples. A test may be structured so that an individual patient sample may be viewed with these populations in mind and allocated to one population or the other, or a mixture of both and subsequently to use this correlation to patient management, therapy, prognosis, etc.

In one aspect of the disclosure, data generated from the methods and kit tests described herein are used with visualizing means is capable of indicating whether the quantity of said one or more markers or fragments in the sample is above or below a certain threshold level or whether the quantity of said one or more markers or fragments in the sample deviates or not from a reference value of the quantity of said one or more markers or fragments, said reference value representing a known diagnosis, prediction or prognosis of the diseases or conditions as taught herein.

In one aspect of the disclosure, data generated from the methods and kit tests described herein determined as a threshold level is chosen such that the quantity of said one or more markers and/or fragments in the sample above or below (depending on the marker and the disease or condition) said threshold level indicates that the subject has or is at risk of having the respective disease or condition or indicates a poor prognosis for such in the subject, and the quantity of said one or more markers and/or fragments in the sample below or above (depending on the marker and the disease or condition) said threshold level indicates that the subject does not have or is not at risk of having the diseases or conditions as taught herein or indicates a good prognosis for such in the subject.

In one aspect of the disclosure, data generated from the methods and kit test described herein determined a relative quantity of a nucleic acid molecule or an analyte in a sample may be advantageously expressed as an increase or decrease or as a fold-increase or fold-decrease relative to said another value, such as relative to a reference value, weight or rank as taught herein. Performing a relative comparison between first and second parameters (e.g., first and second quantities) may but need not require to first determine the absolute values of said first and second parameters. For example, a measurement method can produce quantifiable readouts (such as, e.g., signal intensities) for said first and second parameters, wherein said readouts are a function of the value of said parameters, and wherein said readouts can be directly compared to produce a relative value for the first parameter vs. the second parameter, without the actual need to first convert the readouts to absolute values of the respective parameters.

A. Sensitivity and Specificity

Sensitivity and specificity are statistical measures of the performance of a binary classification test. A perfect classification predictor would be described as 100% sensitive (i.e. predicting all people from the sick group as sick) and 100% specific (i.e. not predicting anyone from the healthy group as sick); however, theoretically any classification predictor will possess a minimum error. (Altman D G, Bland J M (1994). “Diagnostic tests Sensitivity and Specificity”. BMJ 308 (6943): 1552 and Loong T (2003). “Understanding sensitivity and specificity with the right side of the brain”. BMJ 327 (7417): 716-719).

In one aspect of the method of the disclosure using all or some or a combination of the biomarkers achieves a sensitivity selected from greater than 60% true positives, 70% true positives, 75% true positives, 85% true positives, 90% true positives, 95% true positives, or 99% true positives for the subject's adenoma or polyp status. In one aspect of the method of the disclosure using all or some or a combination of the biomarkers achieves a specificity selected from greater than 60% true negatives, 70% true negatives, 75% true negatives, 85% true negatives, 90% true negatives, 95% true negatives, or 99% true negatives for the subject's adenoma, cancer, or polyp status. In one aspect of the method of the disclosure using all or some or a combination of the biomarkers the presence of absence of colorectal carcinoma is excluded or is not determined. In one aspect of the method of the disclosure the presence of absence of the adenoma, cancer, or polyp status is confirmed by additional tests such as a colonoscopy, other imaging method or diagnostic test or surgery. In one aspect of the method of the disclosure using all or some or a combination of the biomarkers achieves a sensitivity and specificity selected from greater than 70% true positives and less than 30% true negatives, 75% true positives and less than 25% true negatives, 85% true positives and less than 15% true negatives, 90% true positives and less than 10% true negatives, 95% true positives and less than 5% true negatives, or 99% true positives for and less than 1% true negatives for the subject's adenoma, cancer, or polyp status.

In one aspect of the method of the disclosure using all or some or a combination of the biomarkers achieves a sensitivity selected from greater than 70% true positives, 75% true positives, 85% true positives, 90% true positives, 95% true positives, or 99% true positives for the subject's presence of absence of colorectal carcinoma. In one aspect of the method of the disclosure using all or some or a combination of the biomarkers achieves a specificity selected from greater than 70% true negatives, 75% true negatives, 85% true negatives, 90% true negatives, 95% true negatives, or 99% true negatives for the subject's presence of absence of colorectal carcinoma. In one aspect of the method of the disclosure does not detect the presence of absence of colorectal carcinoma. In one aspect of the method of the disclosure the presence of absence of colorectal carcinoma is confirmed by additional tests such as a colonoscopy, other imaging method or diagnostic test or surgery. In one aspect of the method of the disclosure using all or some or a combination of the biomarkers achieves a sensitivity and specificity selected from greater than 70% true positives and less than 30% true negatives, 75% true positives and less than 25% true negatives, 85% true positives and less than 15% true negatives, 90% true positives and less than 10% true negatives, 95% true positives and less than 5% true negatives, or 99% true positives for and less than 1% true negatives for the subject's presence of absence of colorectal carcinoma.

In one aspect of the method of the disclosure using all or some or a combination of the biomarkers achieves a sensitivity selected from greater than 70% true positives, 75% true positives, 85% true positives, 90% true positives, 95% true positives, or 99% true positives for the subject's presence of absence of adenomatous polyp or polypoid adenoma. In one aspect of the method of the disclosure using all or some or a combination of the biomarkers achieves a specificity selected from greater than 70% true negatives, 75% true negatives, 85% true negatives, 90% true negatives, 95% true negatives, or 99% true negatives for the subject's presence of absence of adenomatous polyp or polypoid adenoma. In one aspect of the method of the disclosure the adenomatous polyp or polypoid adenoma is confirmed by additional tests such as a colonoscopy, other imaging method or diagnostic test or surgery. In one aspect of the method of the disclosure using all or some or a combination of the biomarkers achieves a sensitivity and specificity selected from greater than 70% true positives and less than 30% true negatives, 75% true positives and less than 25% true negatives, 85% true positives and less than 15% true negatives, 90% true positives and less than 10% true negatives, 95% true positives and less than 5% true negatives, or 99% true positives for and less than 1% true negatives for the subject's presence of absence of adenomatous polyp or polypoid adenoma.

In one aspect of the method of the disclosure using all or some or a combination of the biomarkers achieves a sensitivity selected from greater than 70% true positives, 75% true positives, 85% true positives, 90% true positives, 95% true positives, or 99% true positives for the subject's presence of absence of pedunculated polyps and sessile polyps. In one aspect of the method of the disclosure using all or some or a combination of the biomarkers achieves a specificity selected from greater than 70% true negatives, 75% true negatives, 85% true negatives, 90% true negatives, 95% true negatives, or 99% true negatives for the subject's presence of absence of pedunculated polyps and sessile polyps. In one aspect of the method of the disclosure the of pedunculated polyps and sessile polyps is confirmed by additional tests such as a colonoscopy, other imaging method or diagnostic test or surgery. In one aspect of the method of the disclosure using all or some or a combination of the biomarkers achieves a sensitivity and specificity selected from greater than 70% true positives and less than 30% true negatives, 75% true positives and less than 25% true negatives, 85% true positives and less than 15% true negatives, 90% true positives and less than 10% true negatives, 95% true positives and less than 5% true negatives, or 99% true positives for and less than 1% true negatives for the subject's presence of absence of pedunculated polyps and sessile polyps.

In one aspect of the method of the disclosure using all or some or a combination of the biomarkers achieves a sensitivity selected from greater than 70% true positives, 75% true positives, 85% true positives, 90% true positives, 95% true positives, or 99% true positives for the subject's adenomatous polyp or polypoid adenoma is characterized according to a degree of cell dysplasia or pre-malignancy. In one aspect of the method of the disclosure using all or some or a combination of the biomarkers achieves a specificity selected from greater than 70% true negatives, 75% true negatives, 85% true negatives, 90% true negatives, 95% true negatives, or 99% true negatives for the subject's adenomatous polyp or polypoid adenoma is characterized according to a degree of cell dysplasia or pre-malignancy. In one aspect of the method of the disclosure the adenomatous polyp or polypoid adenoma is characterized according to a degree of cell dysplasia or pre-malignancy confirmed by additional tests such as a colonoscopy, other imaging method or diagnostic test or surgery. In one aspect of the method of the disclosure using all or some or a combination of the biomarkers achieves a sensitivity and specificity selected from greater than 70% true positives and less than 30% true negatives, 75% true positives and less than 25% true negatives, 85% true positives and less than 15% true negatives, 90% true positives and less than 10% true negatives, 95% true positives and less than 5% true negatives, or 99% true positives for and less than 1% true negatives for the subject's adenomatous polyp or polypoid adenoma is characterized according to a degree of cell dysplasia or pre-malignancy.

### VI. Systems

The systems and methods of the present disclosure are enacted on and/or by using one or more computer processor systems. Examples of computer systems of the disclosure are described below. Variations upon the described computer systems are possible so long as they provide the platform for the systems and methods of the disclosure.

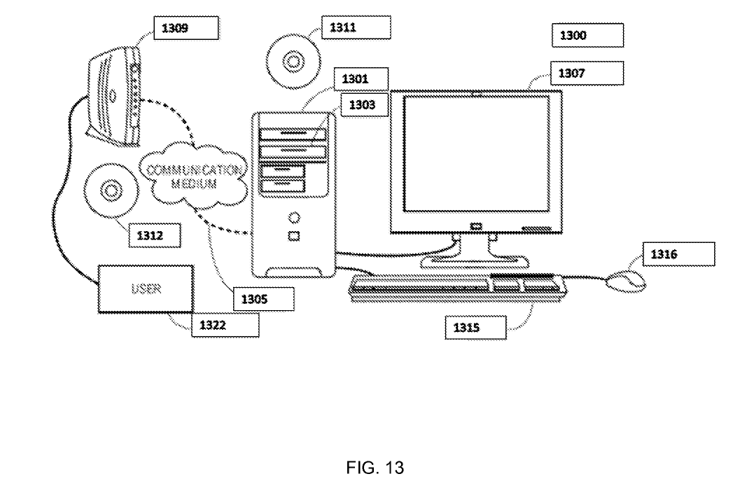

An example of computer system of the disclosure is illustrated in FIG. 13. The computer system 1300 illustrated in FIG. 13 may be understood as a logical apparatus that can read instructions from media 1311 and/or a network port 1305, which can optionally be connected to server 1309 having fixed media 1312. The system, such as shown in FIG. 13 can include a CPU 1301, disk drives 1303, optional input devices such as keyboard 1315 and/or mouse 1316 and optional monitor 1307. Data communication can be achieved through the indicated communication medium to a server at a local or a remote location. The communication medium can include any means of transmitting and/or receiving data. For example, the communication medium can be a network connection, a wireless connection or an internet connection. Such a connection can provide for communication over the World Wide Web. It is envisioned that data relating to the present disclosure can be transmitted over such networks or connections for reception and/or review by a party 1322 as illustrated in FIG. 13.

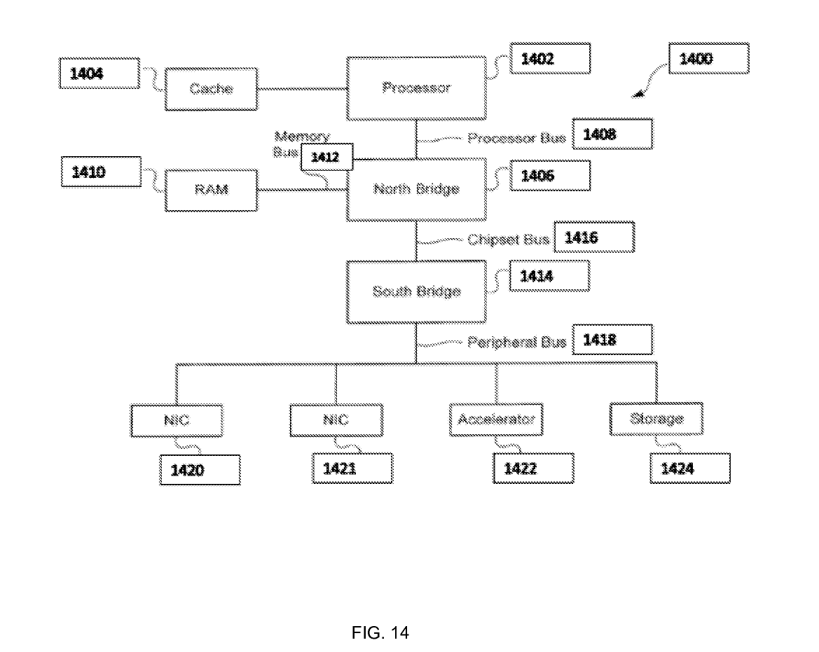

FIG. 14 is a block diagram illustrating an example architecture of a computer system 1400 that can be used in connection with example embodiments of the present disclosure. As depicted in FIG. 14, the example computer system can include a processor 1402 for processing instructions. Non-limiting examples of processors include: Intel Xeon™ processor, AMD Opteron™ processor, Samsung 32-bit RISC ARM 1176JZ(F)-S v1.O™ processor, ARM Cortex-A8 Samsung S5PC100™ processor, ARM Cortex-A8 Apple A4™ processor, Marvell PXA 930™ processor, or a functionally-equivalent processor. Multiple threads of execution can be used for parallel processing. In some aspects of the disclosure, multiple processors or processors with multiple cores can also be used, whether in a single computer system, in a cluster, or distributed across systems over a network comprising a plurality of computers, cell phones, and/or personal data assistant devices.

As illustrated in FIG. 14, a high speed cache 1404 can be connected to, or incorporated in, the processor 1402 to provide a high speed memory for instructions or data that have been recently, or are frequently, used by processor 1402. The processor 1402 is connected to a north bridge 1406 by a processor bus 1408. The north bridge 1406 is connected to random access memory (RAM) 1410 by a memory bus 1412 and manages access to the RAM 1410 by the processor 1402. The north bridge 1406 is also connected to a south bridge 1414 by a chipset bus 1416. The south bridge 1414 is, in turn, connected to a peripheral bus 1418. The peripheral bus can be, for example, PCI, PCI-X, PCI Express, or other peripheral bus. The north bridge and south bridge are often referred to as a processor chipset and manage data transfer between the processor, RAM, and peripheral components on the peripheral bus 1418. In some alternative architectures, the functionality of the north bridge can be incorporated into the processor instead of using a separate north bridge chip. In some aspects of the disclosure, system 100 can include an accelerator card 1422 attached to the peripheral bus 1418. The accelerator can include field programmable gate arrays (FPGAs) or other hardware for accelerating certain processing. For example, an accelerator can be used for adaptive data restructuring or to evaluate algebraic expressions used in extended set processing.

Software and data are stored in external storage 1424 and can be loaded into RAM 1410 and/or cache 1404 for use by the processor. The system 1400 includes an operating system for managing system resources; non-limiting examples of operating systems include: Linux, Windows™, MACOS™, BlackBerry OS™, iOS™, and other functionally-equivalent operating systems, as well as application software running on top of the operating system for managing data storage and optimization in accordance with example embodiments of the present disclosure.

In this example, system 1400 also includes network interface cards (NICs) 1420 and 1421 connected to the peripheral bus for providing network interfaces to external storage, such as Network Attached Storage (NAS) and other computer systems that can be used for distributed parallel processing.

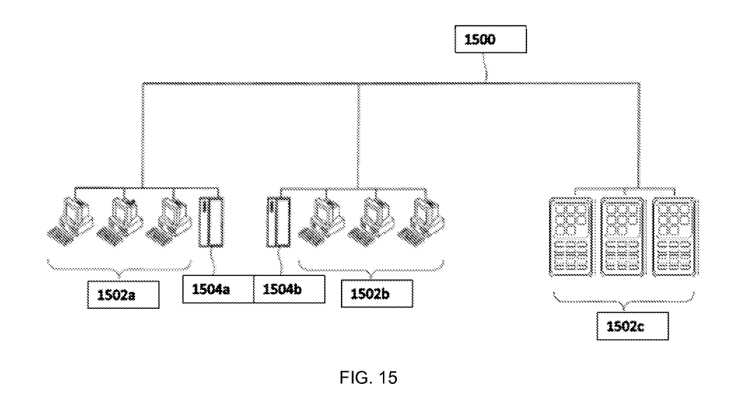

FIG. 15 is a diagram showing a network 1500 with a plurality of computer systems 1502a, and 1502b, a plurality of cell phones and personal data assistants 1502c, and Network Attached Storage (NAS) 1504a, and 1504b. In example embodiments, systems 1502a, 1502b, and 1502c can manage data storage and optimize data access for data stored in Network Attached Storage (NAS) 1504a and 1504b. A mathematical model can be used for the data and be evaluated using distributed parallel processing across computer systems 1502a and 1502b and cell phone and personal data assistant systems 1502c. Computer systems 1502a, and 1502b, and cell phone and personal data assistant systems 1502c can also provide parallel processing for adaptive data restructuring of the data stored in Network Attached Storage (NAS) 1504a and 1504b. A wide variety of other computer architectures and systems can be used in conjunction with the various embodiments of the present disclosure. For example, a blade server can be used to provide parallel processing. Processor blades can be connected through a back plane to provide parallel processing. Storage can also be connected to the back plane or as Network Attached Storage (NAS) through a separate network interface.

In some example embodiments, processors can maintain separate memory spaces and transmit data through network interfaces, back plane or other connectors for parallel processing by other processors. In other embodiments, some or all of the processors can use a shared virtual address memory space.

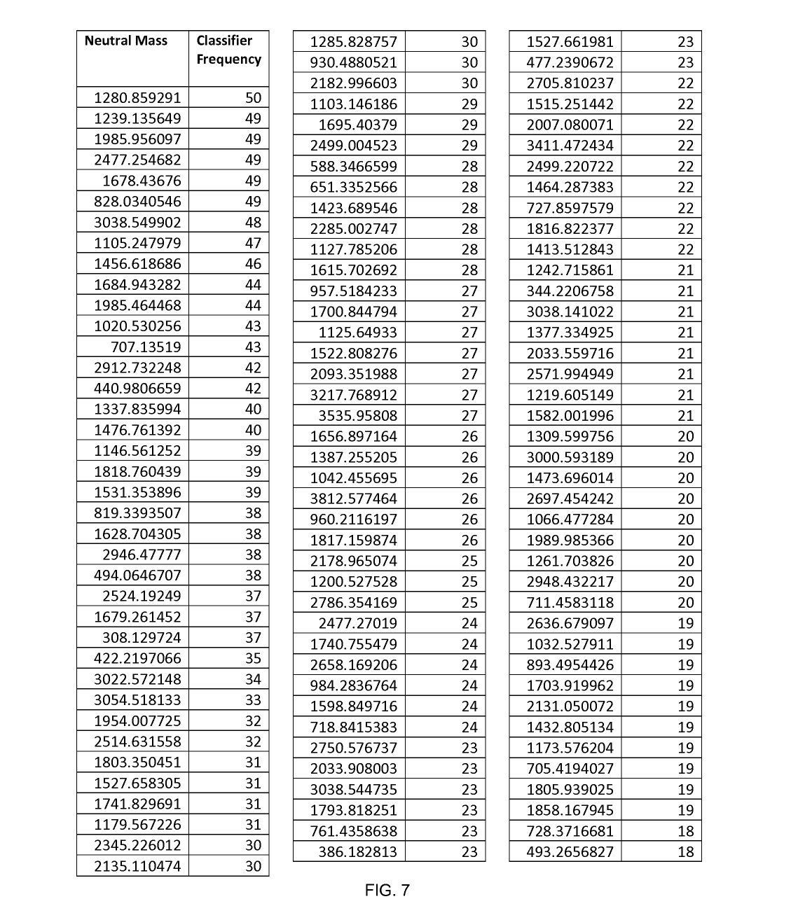

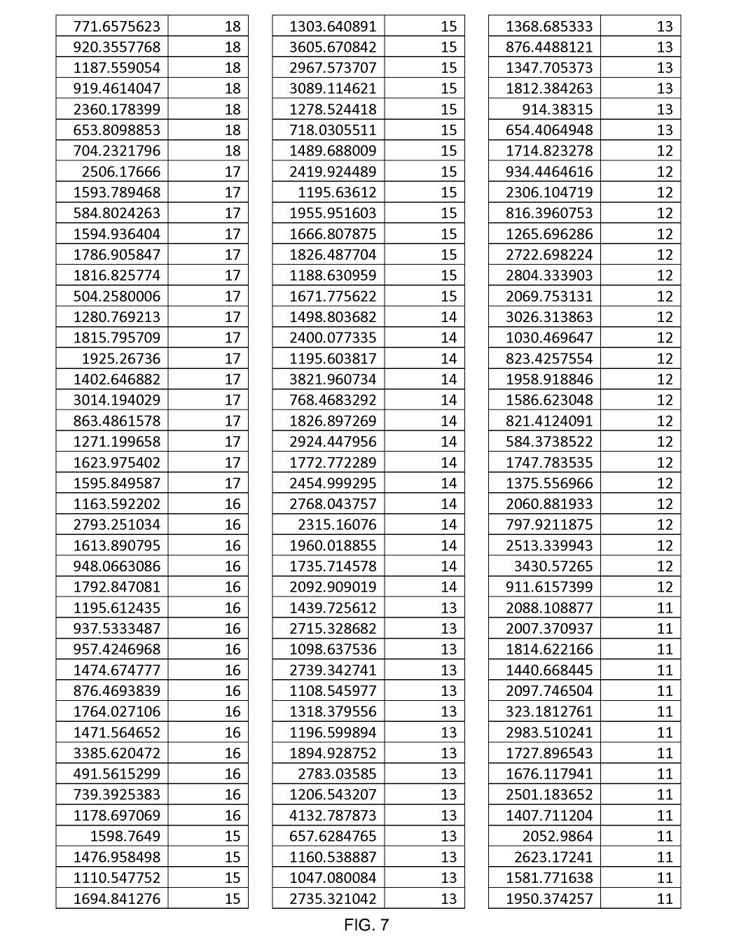

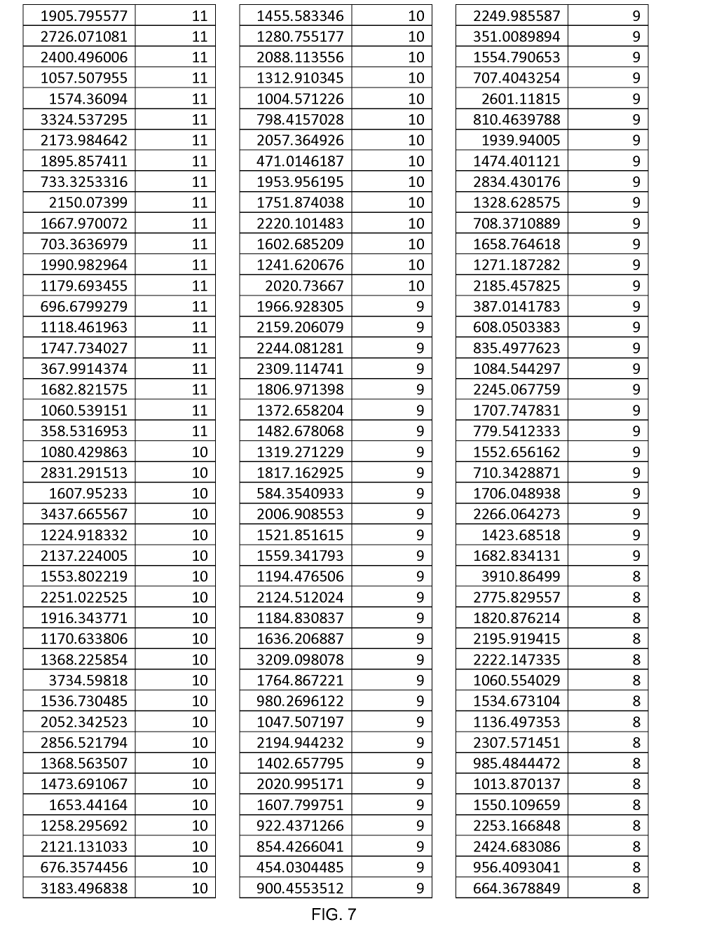

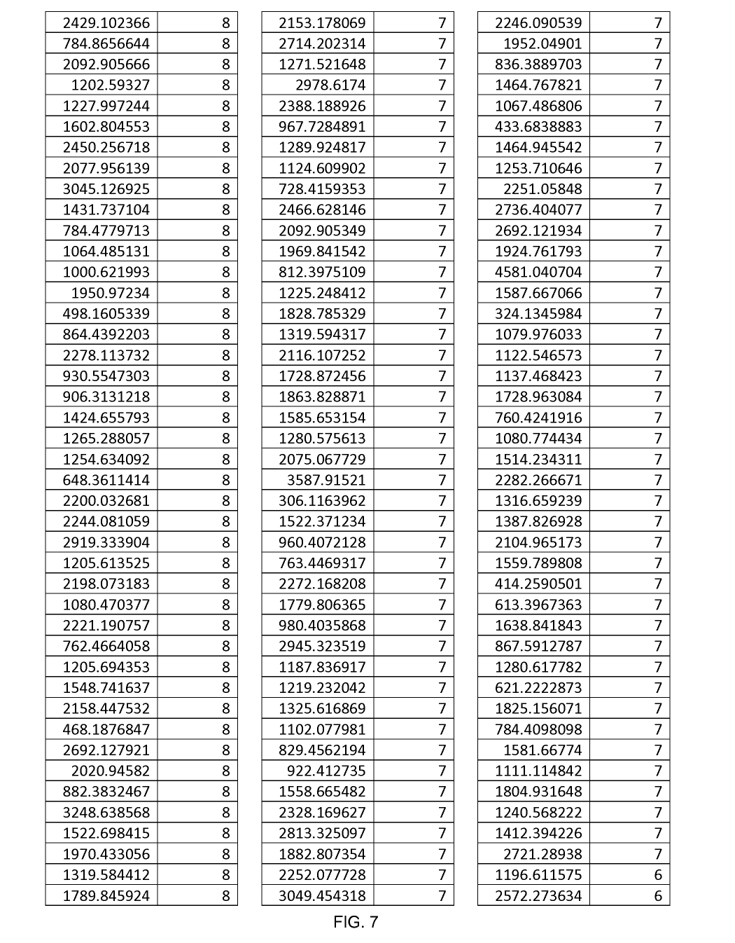

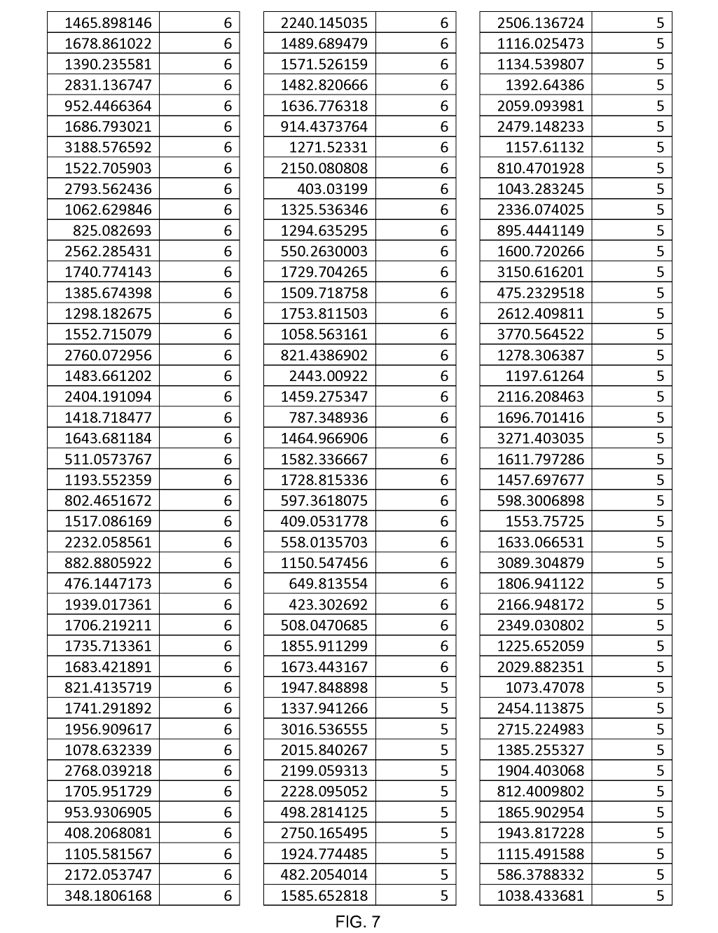

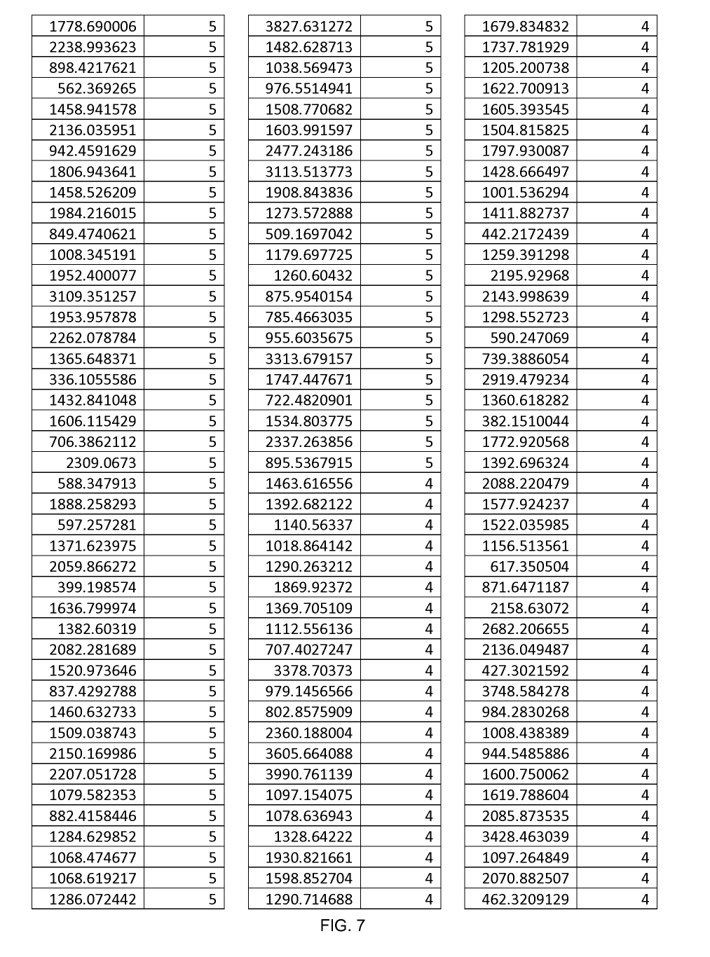

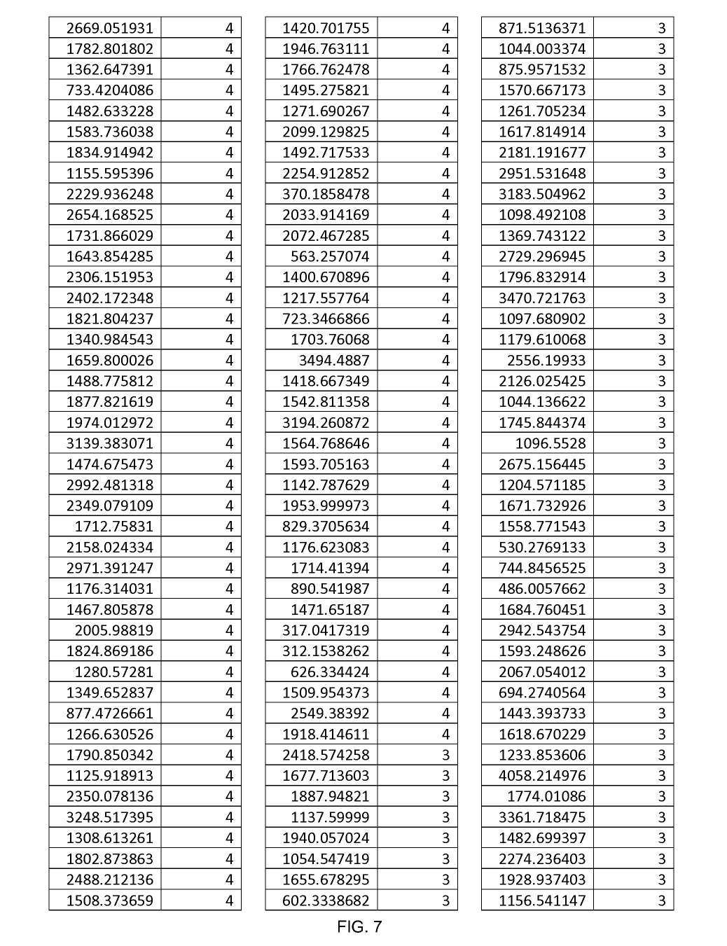

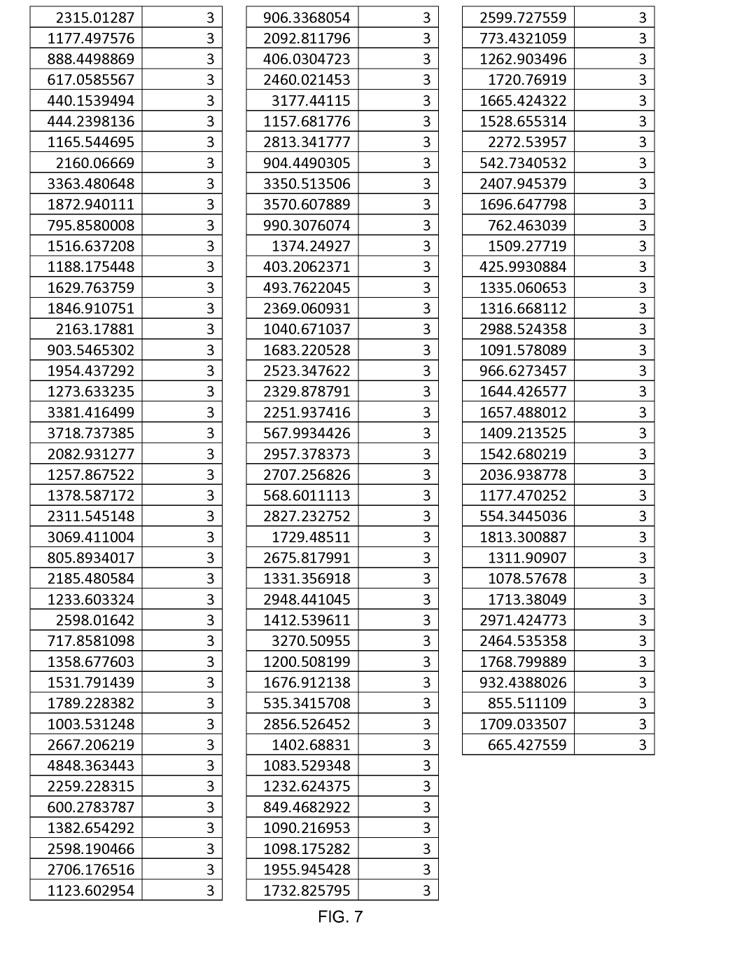

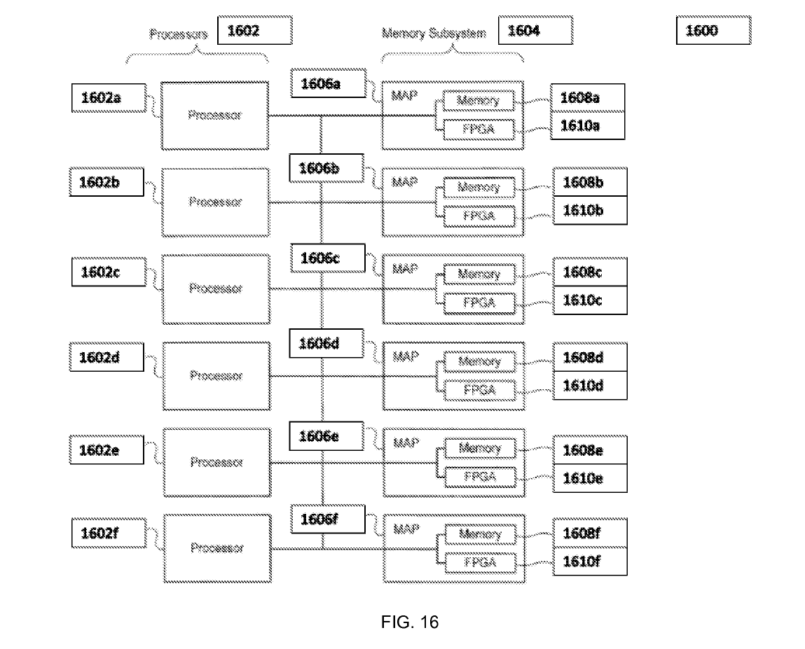

FIG. 16 is a block diagram of a multiprocessor computer system 1600 using a shared virtual address memory space in accordance with an example embodiment. The system includes a plurality of processors 1602a-f that can access a shared memory subsystem 1604. The system incorporates a plurality of programmable hardware memory algorithm processors (MAPs) 160 FIG. 7-f in the memory subsystem 1604. Each MAP 1606a-f can comprise a memory 1608a-f and one or more field programmable gate arrays (FPGAs) 1610a-f. The MAP provides a configurable functional unit and particular algorithms or portions of algorithms can be provided to the FPGAs 1610a-f for processing in close coordination with a respective processor. For example, the MAPs can be used to evaluate algebraic expressions regarding the data model and to perform adaptive data restructuring in example embodiments. In this example, each MAP is globally accessible by all of the processors for these purposes. In one configuration, each MAP can use Direct Memory Access (DMA) to access an associated memory 1608a-f, allowing it to execute tasks independently of, and asynchronously from, the respective microprocessor 1602a-f. In this configuration, a MAP can feed results directly to another MAP for pipelining and parallel execution of algorithms. The disclosure envisions a computer-readable storage medium for example, a CD-ROM, memory key, flash memory card, diskette or other tangible medium having stored thereon a program which, when executed in a computing environment, provides for implementation of custom algorithms to carry out all or a portion of the results of a predictive likelihood or assessment of the provided biological sample as described by the methods of the disclosure. In various embodiments, the computer-readable storage medium is non-transitory.

The systems and methods of the invention integrate one or more pieces of laboratory equipment.

In some embodiments, the integration is performed at a Laboratory Information Management System (LIMS) or lower level. A computer system, may run multiple pieces of laboratory equipment. Software and hardware for laboratory applications may be integrated using the methods and systems of the invention. In various embodiments, similar components with shared functions are repeated in multiple pieces of laboratory equipment.

Computer systems may control multiple components in various pieces of equipment, thus creating new combination of available components. In another example, computer systems of the invention can control mass spectrometry, plate handling, liquid chromatographers, by controlling pumps, sensors, or other components within this piece of laboratory equipment. Software can be provided by anyone, including an independent laboratory end user or any other suitable user. Uses of LIMS in integrated laboratory systems are further described in U.S. Pat. No. 7,991,560, which is herein incorporated by reference in its entirety.

In aspects where the kit provides the computer-readable medium it will contain a complete program for carrying out the methods of the disclosure. The program includes program instructions for collecting, analyzing and generating output, and generally includes computer readable code and devices for interacting with a user as described herein, processing that data in conjunction with analytical information, and generating unique printed or electronic media for that user.

In other aspects the kit provides limited computer-readable medium that runs only portions of the methods of the disclosure. In this aspect the kit provides a program which provides data input from the user and for transmission of data input by the user (e.g., via the internet, via an intranet, etc.) to a computing environment at a remote site such as a server, on which the custom mathematical algorithms of the disclosure will be conducted. Processing or completion of processing of the data provided by the user is carried out at the remote site and the server will also function to generate a report. After review of the report, and completion of any needed manual intervention to provide a complete report, the complete report is then transmitted back to the user as an electronic report or printed report.

The storage medium containing a program according to the disclosure can be packaged with instructions for program installation and use or a web address where such instructions may be obtained.

### VII. Reports

When the methods of the disclosure are used for commercial diagnostic purposes such as in the medical field, generally a report or summary of information obtained from the methods will be generated.

A report or summary of the methods may include information concerning expression levels of one or more genes or proteins, classification of the polyp or tumor, the patient's risk level, such as high, medium or low, the patient's prognosis, treatment options, treatment recommendations, biomarker expression and how biomarker levels were determined, biomarker profile, clinical and pathologic factors, and/or other standard clinical information of the patients or of a population group relevant to the patient's disease state.

The methods and reports can stored in a database. The method can create a record in a database for the subject and populate the record with data. The report may be a paper report, an auditory report, or an electronic record. The report may be displayed and/or stored on a computing device (e.g., handheld device, desktop computer, smart device, website, etc.). It is contemplated that the report is provided to a physician and/or the patient. The receiving of the report can further include establishing a network connection to a server computer that includes the data and report and requesting the data and report from the server computer.

In another aspect the present disclosure provides methods of producing reports that include biomarker information about a biological sample obtained from a subject that includes the steps of determining sample's biomarker profile expression levels of the one or more biomarkers: SCDC26 (CD26), CEA molecule 5 (CEACAM5), CA195 (CCR5), CA19-9, M2PK (PKM2), TIMP1, P-selectin (SELPLG), VEGFA, HcGB (CGB), VILLIN, TATI (SPINK1), A-L-fucosidase (FUCA2), ANXA5, GAPDH, PKM2, ANXA4, GARS, RRBP1, KRT8, SYNCRIP, S100A9, ANXA3, CAPG, HNRNPF, PPA1, NME1, PSME3, AHCY, TPT1, HSPB1, and RPSA, and/or the proteins in FIG. 9, or their modified version or one of their binding partners and creating a report summarizing said their expression levels. In some aspects the report may further include a classification of a subject into a risk group such as “low-risk”, “medium-risk”, or “high-risk”. In various embodiments, groupings of two, three, four, five, six, seven, eight, nine, ten, eleven, and all twelve of the above proteins are included. Such groupings may exclude additional proteins, or may further comprise additional proteins.

In one aspect of the method, if increased expression of one or more biomarkers: SCDC26 (CD26), CEA molecule 5 (CEACAM5), CA195 (CCR5), CA19-9, M2PK (PKM2), TIMP1, P-selectin (SELPLG), VEGFA, HcGB (CGB), VILLIN, TATI (SPINK1), A-L-fucosidase (FUCA2), ANXA5, GAPDH, PKM2, ANXA4, GARS, RRBP1, KRT8, SYNCRIP, S100A9, ANXA3, CAPG, HNRNPF, PPA1, NME1, PSME3, AHCY, TPT1, HSPB1, and RPSA, and/or the proteins in FIG. 9 or their modified version or one of their binding partners, is determined, said report includes a prediction that said subject has an increased likelihood of having a colon polyp. In various embodiments, groupings of two, three, four, five, six, seven, eight, nine, ten, eleven, and all twelve of the above proteins are included. Such groupings may exclude additional proteins, or may further comprise additional proteins.

In another aspect of the method, if increased expression of one or more biomarkers: SCDC26 (CD26), CEA molecule 5(CEACAM5), CA195 (CCR5), CA19-9, M2PK (PKM2), TIMP1, P-selectin (SELPLG), VEGFA, HcGB (CGB), VILLIN, TATI (SPINK1), A-L-fucosidase (FUCA2), ANXA5, GAPDH, PKM2, ANXA4, GARS, RRBP1, KRT8, SYNCRIP, S100A9, ANXA3, CAPG, HNRNPF, PPA1, NME1, PSME3, AHCY, TPT1, HSPB1, and RPSA, and/or the proteins in FIG. 9 or their modified version or one of their binding partners, is determined, said report includes a prediction that said subject has an decreased likelihood of having a colon polyp. In various embodiments, groupings of two, three, four, five, six, seven, eight, nine, ten, eleven, and all twelve of the above proteins are included. Such groupings may exclude additional proteins, or may further comprise additional proteins.

In one aspect the report includes information to support a treatment recommendation for said patient. For example, the information can include a recommendation for ordering one or more, diagnostic tests, colonoscopy, surgery, therapeutic treatments and taking no further medical action, a likelihood of benefit score from such treatments, or other such data. In some embodiments, the report further includes a recommendation for a treatment modality for said patient

In one aspect of the disclosure the report is in paper form. In one aspect of the disclosure the report is electronic form such a CD-ROM, flash drive, other electronic storage devices known in the art. In another aspect of the disclosure the electronic report is downloaded from a wired or wireless network to a secondary computer device such as laptop, mobile phone or tablet.

In one aspect the report indicates that if increased expression of one or more biomarkers: SCDC26 (CD26), CEA molecule 5 (CEACAM5), CA195 (CCR5), CA19-9, M2PK (PKM2), TIMP1, P-selectin (SELPLG), VEGFA, HcGB (CGB), VILLIN, TATI (SPINK1), A-L-fucosidase (FUCA2), ANXA5, GAPDH, PKM2, ANXA4, GARS, RRBP1, KRT8, SYNCRIP, S100A9, ANXA3, CAPG, HNRNPF, PPA1, NME1, PSME3, AHCY, TPT1, HSPB1, and RPSA, and/or the proteins in FIG. 9 or their modified version or one of their binding partners, is determined, the report includes a prediction that said subject has an increased likelihood of recurrence of colon polyp or tumor at 5-10 years. In various embodiments, groupings of two, three, four, five, six, seven, eight, nine, ten, eleven, and all twelve of the above proteins are included. Such groupings may exclude additional proteins, or may further comprise additional proteins.

In another aspect the report indicates that if increased expression of one or more one or more of or biomarkers: SCDC26 (CD26), CEA molecule 5 (CEACAM5), CA195 (CCR5), CA19-9, M2PK (PKM2), TIMP1, P-selectin (SELPLG), VEGFA, HcGB (CGB), VILLIN, TATI (SPINK1), A-L-fucosidase (FUCA2), ANXA5, GAPDH, PKM2, ANXA4, GARS, RRBP1, KRT8, SYNCRIP, S100A9, ANXA3, CAPG, HNRNPF, PPA1, NME1, PSME3, AHCY, TPT1, HSPB1, and RPSA, and/or the proteins in FIG. 9 or their modified version or one of their binding partners, is determined, the report includes a prediction that said subject has a decreased likelihood colon polyp or tumor recurrence at 5-10 years. In various embodiments, groupings of two, three, four, five, six, seven, eight, nine, ten, eleven, and all twelve of the above proteins are included. Such groupings may exclude additional proteins, or may further comprise additional proteins.

In some aspects of the disclosure, the report further includes a recommendation for a treatment modality for said patient for treatment management of colon disease. Treatment management options can include but are not limited to, other diagnostic tests such as, colonoscopy, flex sigmoidscopy, CT colonography, stool test, fecal test, further treatment by a therapeutic agent, surgery intervention, and taking no further action.

The present disclosure also provides methods of preparing a personal biomarker profile for a patient by a) determining the normalized expression levels of at least one or more of the SCDC26 (CD26), CEA molecule 5 (CEACAM5), CA195 (CCR5), CA19-9, M2PK (PKM2), TIMP1, P-selectin (SELPLG), VEGFA, HcGB (CGB), VILLIN, TATI (SPINK1), A-L-fucosidase (FUCA2), ANXA5, GAPDH, PKM2, ANXA4, GARS, RRBP1, KRT8, SYNCRIP, S100A9, ANXA3, CAPG, HNRNPF, PPA1, NME1, PSME3, AHCY, TPT1, HSPB1, and RPSA, and/or the proteins in FIG. 9 or their modified version, or its expression product, in a biological sample obtained from a subject t; and (b) creating a report summarizing the data obtained by the gene expression analysis. In various embodiments, groupings of two, three, four, five, six, seven, eight, nine, ten, eleven, and all twelve of the above proteins are included. Such groupings may exclude additional proteins, or may further comprise additional proteins.

### VIII. Kits

The materials for use in the methods of the present disclosure are suited for preparation of kits produced in accordance with well known procedures. The kits provided by the present disclosure marketed to health care providers, including physicians, clinical laboratory scientists, nurses, pharmacists, formulary official or directly to the consumer.

Kits can often comprise insert materials, compositions, reagents, device components, and instructions on how to perform the methods or test on a particular biological sample type. The kits can further comprise reagents to enable the detection of biomarker by various assays types such as ELISA assay, immunoassay, protein chip or microarray, DNA/RNA chip or microarray, RT-PCR, nucleic acid sequencing, mass spectrometry, immunohistochemistry, flow cytometry, or high content cell screening.

The present disclosure provides for compositions such as binding agents capable of specifically binding to any one or more the biomarkers, peptides, polypeptides or proteins and fragments thereof as taught herein. Binding agents may include an antibody, aptamer, photoaptamer, protein, peptide, peptidomimetic or a small molecule. Binding agent provide by the present disclosure include both specific-binding agents that act by binding to one or more desired molecules or analytes, such as to one or more proteins, polypeptides or peptides of interest or fragments thereof substantially to the exclusion of other molecules which are random or unrelated, and optionally substantially to the exclusion of other molecules that are structurally similar or related. The term “specifically bind” does not necessarily require that an agent binds exclusively to its intended target(s). For example, an agent may be said to specifically bind to protein(s) polypeptide(s), peptide(s) and/or fragment(s) thereof of interest if its affinity for such intended target(s) under the conditions of binding is at least about 2-fold greater, preferably at least about 5-fold greater, more preferably at least about 10-fold greater, yet more preferably at least about 25-fold greater, still more preferably at least about 50-fold greater, and even more preferably at least about 100-fold or more greater, than its affinity for a non-target molecule.

Preferably, the binding agent may bind to its intended target(s) with affinity constant (KA) of such binding KA 1×106 M−1, more preferably KA 1×107 M−1, yet more preferably KA 1×108 M−1, even more preferably KA 1×109 M−1, and still more preferably KA 1×101° M−1 or KA 1×1011 M−1, wherein KA=[SBA_T]/[SBA][1], SBA denotes the specific-binding agent, T denotes the intended target. Determination of KA can be carried out by methods known in the art, such as for example, using equilibrium dialysis and Scatchard plot analysis.

In some applications of the methods and kits the binding agent will be an immunologic binding agent, such as an antibody. Examples of antibodies that can be used with the present disclosure include polyclonal and monoclonal antibodies as well as fragments thereof are well known in the art. Additional examples of antibodies that can be used this is methods and kit of the present disclosure include multivalent (e.g., 2-, 3- or more-valent) and/or multi-specific antibodies (e.g., bi- or more-specific antibodies) formed from at least two intact antibodies, and antibody fragments insofar they exhibit the desired biological activity (particularly, ability to specifically bind an antigen of interest), as well as multivalent and/or multi-specific composites of such fragments.

An antibody may be any of IgA, IgD, IgE, IgG and IgM classes, and preferably IgG class antibody. An antibody may be a polyclonal antibody, e.g., an antiserum or immunoglobulins purified there from (e.g., affinity-purified). An antibody may be a monoclonal antibody or a mixture of monoclonal antibodies. Monoclonal antibodies can target a particular antigen or a particular epitope within an antigen with greater selectivity and reproducibility. By means of example and not limitation, monoclonal antibodies may be made by the hybridoma method first described by Kohler et al. 1975 (Nature 256: 495), or may be made by recombinant DNA methods (e.g., as in U.S. Pat. No. 4,816,567). Monoclonal antibodies may also be isolated from phage antibody libraries using techniques as described by Clackson et al. 1991 (Nature 352: 624-628) and Marks et al. 1991 MolBiol 222: 581-597), for example.

Antibody binding agents may be antibody fragments. “Antibody fragments” comprise a portion of an intact antibody, comprising the antigen-binding or variable region thereof. Examples of antibody fragments include Fab, Fab′, F(ab′)2, Fv and scFv fragments; diabodies; linear antibodies; single-chain antibody molecules; and multivalent and/or multispecific antibodies formed from antibody fragment(s), e.g., dibodies, tribodies, and multibodies. The above designations Fab, Fab′, F(ab′)2, Fv, scFv etc. are intended to have their art-established meaning

Methods of producing polyclonal and monoclonal antibodies as well as fragments thereof are well known in the art, as are methods to produce recombinant antibodies or fragments thereof (see for example, Harlow and Lane, “Antibodies: A Laboratory Manual”, Cold Spring Harbour Laboratory, New York, 1988; Harlow and Lane, “Using Antibodies: A Laboratory Manual”, Cold Spring Harbour Laboratory, New York, 1999, ISBN 0879695447; “Monoclonal Antibodies: A Manual of Techniques”, by Zola, ed., CRC Press 1987, ISBN 0849364760; “Monoclonal Antibodies: A Practical Approach”, by Dean & Shepherd, eds., Oxford University Press 2000, ISBN 0199637229; Methods in Molecular Biology, vol. 248: “Antibody Engineering: Methods and Protocols”, Lo, ed., Humana Press 2004, ISBN 1588290921).

Antibodies of the present disclosure can originate from or comprising one or more portions derived from any animal species, preferably vertebrate species, including, e.g., birds and mammals. Without limitation, the antibodies may be chicken, chicken egg, turkey, goose, duck, guinea fowl, quail or pheasant. Also without limitation, the antibodies may be human, murine (e.g., mouse, rat, etc.), donkey, rabbit, goat, sheep, guinea pig, camel (e.g., Camelus bactrianus and Camelus dromaderius), llama (e.g., Lama paccos, Lama glama or Lama vicugna) or horse.

The disclosure also provided for an antibody to the biomarkers provided herein may include one or more amino acid deletions, additions and/or substitutions (e.g., conservative substitutions), insofar such alterations preserve its binding of the respective antigen. An antibody may also include one or more native or artificial modifications of its constituent amino acid residues (e.g., glycosylation, etc.).

The antibodies provide by the present disclosure are not limited to antibodies generated by methods comprising immunization but also includes any polypeptide, e.g., a recombinantly expressed polypeptide, which is made to encompass at least one complementarity-determining region (CDR) capable of specifically binding to an epitope on an antigen of interest. Hence, the terms antibody or immunologic binding agent applies to such molecules regardless whether they are produced in vitro or in vivo.

Antibody or immunologic binding agents, peptides, polypeptides, proteins, biomarkers etc. in the present kits may be in various forms, e.g., lyophilised, free in solution or immobilised on a solid phase. Antibody or immunologic binding agents may be, e.g., provided in a multi-well plate or as an array or microarray, or they may be packaged separately and/or individually. The may be suitably labeled to detection as taught herein. Kits provide herein may be particularly suitable for performing the assay methods of the disclosure, such as, e.g., immunoassays, ELISA assays, mass spectrometry assays, flow cytometry and the like.

In disclosure provide for kits to be delivered and used by qualified clinical scientists. In such kit the disclosure provides for kits comprised of various agents, which may include antibodies read-out detection antibodies that recognized of one or more of the disclosed biomarkers, gene-specific or gene-selective probes and/or primers, for quantitating the expression of one or more of the disclosed biomarkers, modified form or binding partners of the biomarker for predicting colon tumor status or response to treatment.

The kits may be further comprised of containers (including microtiter plates suitable for use in an automated implementation of the method), pre-fabricated biochips, buffers, the appropriate regents antibodies, probes, enzymes to conduct the assay. In some aspects of the disclosure kits may contain reagents for the extraction of protein and nucleic acid from biological samples, and/or reagents for DNA or RNA amplification or protein fractionation or purification and a capture biochip that detects the biomarkers The reagent(s) in the kit will have with an identifying description or label or instructions relating to their use and steps to conduct the assay. In addition, the kits can be further comprised of instructions relating to their use in the methods used to determine the likelihood of colon polyp/tumor status and recurrence and treatment response or a computer-readable storage medium can also be provided in combination to determine the likelihood of colon polyp/tumor status and recurrence and treatment response.

A kit can further comprise a software package for data analysis which can include reference biomarker profiles for comparison. In some applications, the kits' software package including connection to a central server to conduct for data analysis and where a report with recommendation on disease state, treatment suggestions, or recommendation for treatments or procedures for disease management.

The report provide with the kit can be a paper or electronic report. It can be generated by computer software provided with the kit, or by a computer sever which the user uploads to a website wherein the computer server generates the report.

In some aspects of the disclosure kits may contain mathematical algorithms used to estimate or quantify prognostic, diagnostic, clinical status, or predictive information as components of kits. In some aspects this will delivered though computer-readable storage media and other aspects of the disclosure this might be given by supplying the user with a password to access a computer server containing the logic to run the mathematical algorithms.

The kit can be packaged in any suitable manner, typically with all elements in a single container along with a sheet of printed instructions for carrying out the method or test.

In disclosure provide for kits to be delivered to a physician. The kit for this purpose would in include an electronic or written document for the physician to provide medical information and bar-code labels to adhere to sterile receptacle containers containing the biological samples and optional fixative/preservative regents. In some aspects such a kit will include mailing instruction and supplies to be sent by mail for processing by the methods provided herein.

### EXAMPLES

### Example 1

### Identification of Adenoma or Polyp Status in Individuals with Negative Diagnosis from Colonoscopy

Whole serum from patients with a negative diagnosis of adenoma or polyps based on colonoscopy is tested for the presence of absence of colon polyps using the validated biomarker classifier. Data is analyzed from each site's samples independently (i.e., the validation data set is not used for training or testing in discovery cross-validation) and then is evaluated for overlap between the results. LC-MS/MS analysis is performed on proteins and/or peptides of the classifier in TABLE E1.

Biomarkers are identified. For example, biomarker collections are shown in TABLE E1 and TABLE E2, and FIG. 7.

TABLE E1

| | Name (alter- | |
|---|---|---|
| No. | native name) | |
| 1 | SCDC26 (CD26) | Dipeptidyl peptidase 4 soluble form |
| 2 | CEA molecule 5 | Carcinoembryonic anitigen-related adhesion |
| | (CEACAM5) | |
| 3 | CA195 (CCR5) | C-C chemokine receptor type 5 |
| 4 | CA19-9 | carbohydrate antigen 19-9 |
| 5 | M2PK (PKM2) | Pyruvate kinase isozymes M1/M2 |
| 6 | TIMP1 | Metalloproteinase inhibitor 1 |
| 7 | P-selectin | P-selectin glycoprotein ligand 1 |
| | (SELPLG) | |
| 8 | VEGFA | Vascular endothelial growth factor A |
| 9 | HcGB (CGB) | Choriogonadotropin subunit beta |
| 10 | VILLIN | Epithelial cell-specific Ca2+-regulated actin |
| 11 | TATI (SPINK1) | Pancreatic secretory tyrpsin inhibitor |
| 12 | A-L-fucosidase | Plasma alpha-L-fucosidase |
| | (FUCA2) | |

TABLE E2

| | Name (alter- | |
|---|---|---|
| No. | native name) | |
| 1 | ANXA5 | Annexin A5 |
| 2 | GAPDH | Glyceraldehyde-3-phosphate dehydrogenase |
| 3 | PKM2 | Pyruvate kinase isozymes M1/M2 |
| 4 | ANXA4 | Annexin A4 |
| 5 | GARS | Glycyl-tRNA synthetase |
| 6 | RRBP1 | Ribosome-binding protein 1 |
| 7 | KRT8 | Keratin, type II cytoskeletal 8 |
| 8 | SYNCRIP | Heterogeneous nuclear ribonucleoprotein Q |
| 9 | S100A9 | S100 A9 Calcium binding protein |
| 10 | ANXA3 | Annexin A3 |
| 11 | CAPG | Macrophage-capping protein |
| 12 | HNRNPF | Heterogeneous nuclear ribonucleoprotein F |
| 13 | PPA1 | Inorganic pyrophosphatase |
| 14 | NME1 | Nucleoside diphosphate kinase A |
| 15 | PSME3 | Proteasome activator complex subunit 3 |
| 16 | AHCY | Adenosylhomocysteinase |
| 17 | TPT1 | Translationally-controlled tumor protein |
| 18 | HSPB1 | Heat shock protein beta-1 |
| 19 | RPSA | 40S ribosomal protein SA |

These values are compared to a control reference value. Finally, the classifier profile is compared to low or no-risk, medium-risk and high-risk classifier profiles, allowing the patient sample to be correlated to the subject's predicted adenoma/polyp status or normal at around 90% or better accuracy rate. See TABLE E3. Alternatively, the clinical test is performed using the biomarker classifier by immunological analysis such as immunoblotting, biochip, immunostaining and/or flow cytometry analysis.

TABLE E3

| | |
|---|---|
| Validation Set | Discovery Set |

| | Normal | Polyps | Normal | Polyps |
|---|---|---|---|---|
| | n = 500 | n = 600 | n = 400 | n = 700 |
| Classified as | 461 | 0 | 387 | 0 |
| normal (non- | | | | |
| polyp) | | | | |
| Classified as | 0 | 543 | 0 | 673 |
| with polyp | | | | |
| Cannot classify | 39 | 57 | 13 | 27 |

### Example 2

### Identification of Recurrence of a Polyp Status in Individuals Who Previously Presented with Colon Polyps

A capture biochip with antibodies that specifically bind to or recognize antigens to the protein biomarker classifier in TABLE E1 and/or TABLE E2 and control references is used to profile antigens in whole serum samples from patients who have presented earlier with a colon polyp tumor.

Samples are screened to determine if the patients had recurrence of a colon polyp or polyp. The chip is incubated with the sample at room temperature to allow antibodies to form a complex of with the antigens in the sample. Next, the chip is washed with a mild detergent solution to remove any proteins or antibodies that are not specifically bound. A secondary antibody-complex with a detection reagent is added and allowed to bind the chip, and is washed with a mild detergent. Proteins are quantified using a reader such as a CCD camera. Finally, the classifier profile from the biochip read-out is to compared to low or no-risk, medium-risk and high-risk recurrence classifiers profiles to determine the patient's recurrence status.

### Example 3A

In this study, blood was collected from patients who were about to undergo colonoscopy. Quantitative data on the profiles of protein-based molecular features present in plasma were collected using a tandem mass spectrometry-based process, and the data were used to identify features that comprise classifiers with the ability to predict the outcome of the colonoscopy procedure.

Study Design and Patient Sample Collection

In order to correlate plasma protein profiles with patient colonoscopy outcomes, blood samples were collected from patients presenting for colonoscopies on the day of their procedures. Inclusion criteria required that the patient be equal to or greater than 18 years of age and be willing and able to sign an informed consent. This was an “all comers” study in which patients could be undergoing the procedure as a recommended, routine screen, as a precaution due to prior personal or family history, or as a follow up to personal health symptoms.

After the routine preparation for colonoscopy that included overnight fasting, liquid-type constraints, and bowel prep to remove fecal matter, a blood sample was drawn into a plasma collection device that included EDTA as an anti-coagulant. The blood sample was mixed, centrifuged to separate plasma as per the manufacturer's instructions, and the separated plasma was collected and frozen at −80 C within four hours.

In addition to the plasma sample, patient clinical data such as age, weight, gender, ethnicity, current medications and indications, and personal and family health history were collected as were the colonoscopy procedure report and the pathology report on any collected and examined tissues. More than 500 patient samples were collected. Patient demographic data is provided in TABLE E4, TABLE E5, and TABLE E6.

TABLE E4

| | | | Disease | | | | |
|---|---|---|---|---|---|---|---|
| | | Control | | Adenoma | | | |
| | Excluded | Normal | Polyp | and Polyp | Adenoma | Total | % Total |
| Total | 3 | 73 | 20 | 7 | 49 | 152 | 100.00% |
| Routine Visit | 0 | 37 | 6 | 1 | 22 | 66 | 43.42% |
| History | 0 | 14 | 10 | 5 | 15 | 44 | 28.95% |
| Symptoms | 3 | 22 | 4 | 1 | 12 | 42 | 27.63% |
| Prior Colonoscopy | 1 | 41 | 13 | 6 | 25 | 86 | 56.58% |
| Male | 2 | 35 | 8 | 4 | 27 | 76 | 50.00% |
| Female | 1 | 38 | 12 | 3 | 22 | 76 | 50.00% |
| African American | 1 | 3 | 2 | 0 | 2 | 8 | 5.26% |
| Asian | 0 | 0 | 0 | 1 | 0 | 1 | 0.66% |
| Caucasian | 2 | 69 | 16 | 6 | 45 | 138 | 90.79% |
| Hispanic | 0 | 1 | 1 | 0 | 2 | 4 | 2.63% |
| Indian | 0 | 0 | 1 | 0 | 0 | 1 | 0.66% |
| Pacific Islander | 0 | 0 | 0 | 0 | 0 | 0 | 0.00% |

TABLE E5

| | Control | Disease |
|---|---|---|
| Female | 38 | 37 |
| Mail | 35 | 39 |
| | | p = 0.6808 |
| Age (average +/− | 58.8 +/− 9.8 | 58.9 +/− 9.6 |
| stdev in years) | | |
| | | p = 0.9305 |
| Routine | 37 | 29 |
| History or symptoms | 36 | 47 |
| | | p = 0.1237 |

p-Values from Chi-Squared Tests of Association

TABLE E6

| | # in | | | | | Chi |
|---|---|---|---|---|---|---|
| | Training | Control | Control | Disease | Disease | Squared |
| Condition or Medication | Set | with | without | with | without | p-value |
| Allergies | 27 | 15 | 58 | 12 | 64 | 0.450942 |
| Anemia | 10 | 6 | 67 | 4 | 72 | 0.470814 |
| AnxietyDisorder | 13 | 8 | 65 | 5 | 71 | 0.343321 |
| Arthritis | 13 | 6 | 67 | 7 | 69 | 0.830237 |
| Asthma | 16 | 5 | 68 | 10 | 66 | 0.199724 |
| Constipation | 12 | 4 | 69 | 7 | 69 | 0.383146 |
| Depression | 32 | 19 | 54 | 13 | 63 | 0.184788 |
| DiabetesTypeII | 25 | 8 | 65 | 15 | 61 | 0.137476 |
| DiverticularDisease | 13 | 8 | 65 | 5 | 71 | 0.343321 |
| GastroesophagealRefluxDiseases(GERD) | 36 | 13 | 60 | 22 | 54 | 0.108432 |
| Hypercholesterolemia | 22 | 11 | 62 | 11 | 65 | 0.918512 |
| HyperlipidemiaDyslipidemia | 45 | 16 | 57 | 27 | 49 | 0.066549 |
| Hypertension | 64 | 29 | 44 | 34 | 42 | 0.535918 |
| Hypothyrodism | 21 | 8 | 65 | 13 | 63 | 0.280525 |
| Insomia | 13 | 8 | 65 | 5 | 71 | 0.343321 |
| IrritableBowelSyndrome(IBS) | 17 | 10 | 63 | 7 | 69 | 0.388888 |
| HCTZHydrochlorothiazide | 14 | 7 | 66 | 6 | 70 | 0.714104 |
| ASAAsprin | 45 | 20 | 53 | 24 | 52 | 0.575854 |
| Albuterol | 12 | 5 | 68 | 7 | 69 | 0.596230 |
| CalciumSupplement | 26 | 10 | 63 | 16 | 60 | 0.236565 |
| FishOil | 23 | 11 | 62 | 12 | 64 | 0.903077 |
| Flovent | 15 | 9 | 64 | 6 | 70 | 0.368360 |
| HormoneReplacementTherapy | 14 | 10 | 63 | 4 | 72 | 0.076930 |
| Ibuprofen | 11 | 6 | 67 | 5 | 71 | 0.701900 |
| Levothyroxine | 18 | 7 | 66 | 11 | 65 | 0.359898 |
| Lipitor | 12 | 4 | 69 | 8 | 68 | 0.256630 |
| Lisinopril | 17 | 4 | 69 | 12 | 64 | 0.041113 |
| Metformin | 14 | 4 | 69 | 9 | 67 | 0.167563 |
| Pravachol | 11 | 3 | 70 | 8 | 68 | 0.132598 |
| Prilosec | 27 | 12 | 61 | 15 | 61 | 0.601195 |
| VitaminC | 12 | 5 | 68 | 7 | 69 | 0.696230 |
| VitaminD | 25 | 11 | 62 | 13 | 63 | 0.735244 |
| VitaminD3 | 10 | 3 | 70 | 7 | 69 | 0.211955 |
| Zocor | 18 | 7 | 66 | 10 | 66 | 0.493048 |

Sample Preparation for Plasma Protein Analysis

152 samples (76 polyp and/or adenoma and 76 control) were selected for classifier analysis. The polyp and/or adenoma group of patients was randomly selected from the larger study cohort and matched for age and gender from controls. Patient plasma protein samples were prepared for LCMS measurement as follows. Plasma samples were thawed from −80 C storage and lipids and particulates were removed by filter centrifugation. The high-abundance proteins in the filtered plasma were removed by immunoaffinity column-based depletion. The lower abundance, flow-through proteins were separated into fractions by reverse-phase HPLC. Selected protein fractions, six per sample, were reduced to peptides by trypsin-TFE digestion, and the resulting peptides were re-suspended in acetonitrile/formic acid LCMS loading buffer.

LCMS Data Acquisition and Protein Molecular Feature Quantification

Re-suspended peptides from several fractions of each patient's plasma sample were injected via UHPLC into a tandem mass spectrometer (Q-TOF) for quantitative analysis. The collected data (retention time, mass/charge ratio, and ion abundance) were analyzed to detect observed peaks referred to as molecular features. A three-dimensional peak integration algorithm determined the relative abundance of the molecular features.

Molecular feature data from multiple patient samples were compared after dataset overlay and alignment using a cubic spline algorithm. Only the features determined to be present in 50% or more of at least one of the patient classes (clean or polyp/adenoma) were considered for further analysis. In the case of missing patient-feature data in this set, feature values were imputed by integrating the raw ion abundance data in the a priori location of the peak as observed in other samples. More than 145,000 molecular features from each of the 152 patient samples comprised the final data set for subsequent classifier analysis.

Data Normalization, Feature Selection and Classifier Assembly

The quantitative data for distinct molecular features derived from a single original neutral mass were combined and summarized. For example, +2 m/z and +3 m/z features from the same parent molecule were combined by summing to a single neutral mass cluster (NMC) value.

Molecular feature data from different samples were normalized by mean adjusting NMCs from samples collected on the same instrument and day of the study. Data acquisition was balanced such that approximately equal numbers of clean and polyp/adenoma samples were evaluated in each instrument-day group. This method is defined as cluster-instrument-day (“CID”) normalization.

Initial analysis of the data suggested that an imbalance in the hormone-replacement therapy status of the female samples might be a confounding factor in classifier building. To eliminate that possibility, molecular features that were suggested to be HRT-related were identified by differential classifier assembly and removed from subsequent analysis.

Only samples with complete data from all experimental fractions were used for analysis. Of the 152 samples originally, measured, 108 complete samples remained. For most of the excluded samples, the QC failure of one or more of the 6 sample fractions resulted in the exclusion.

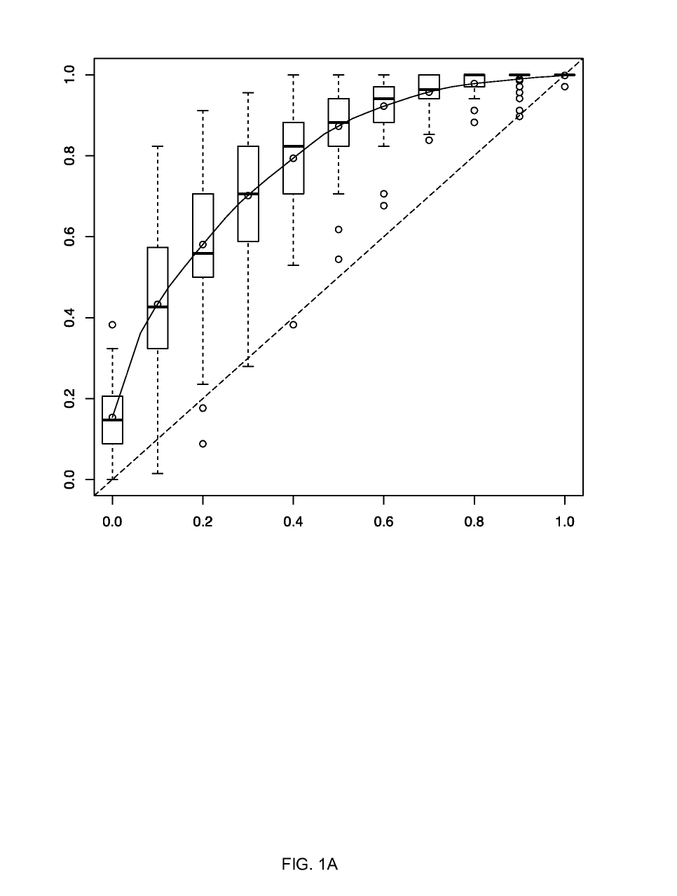

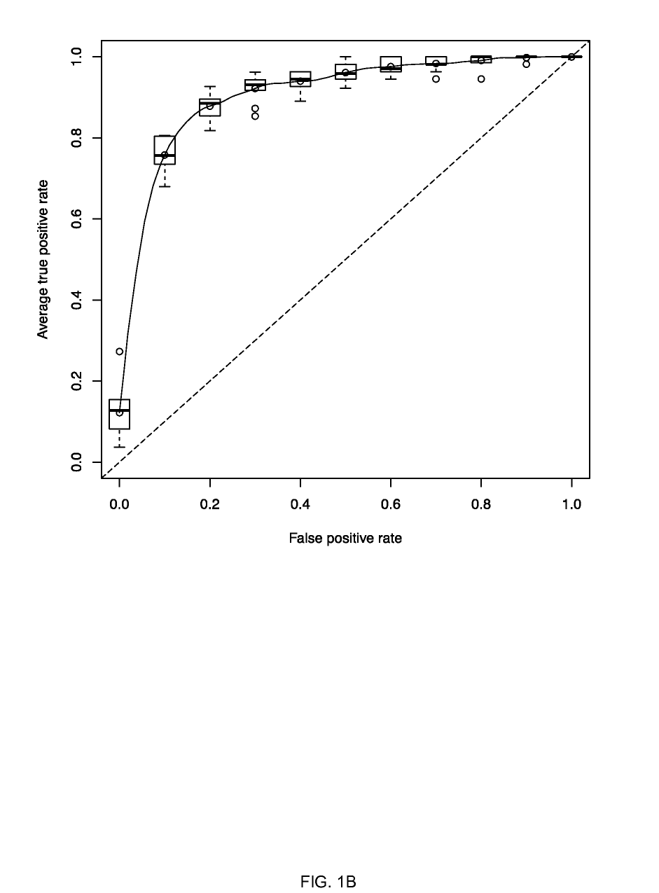

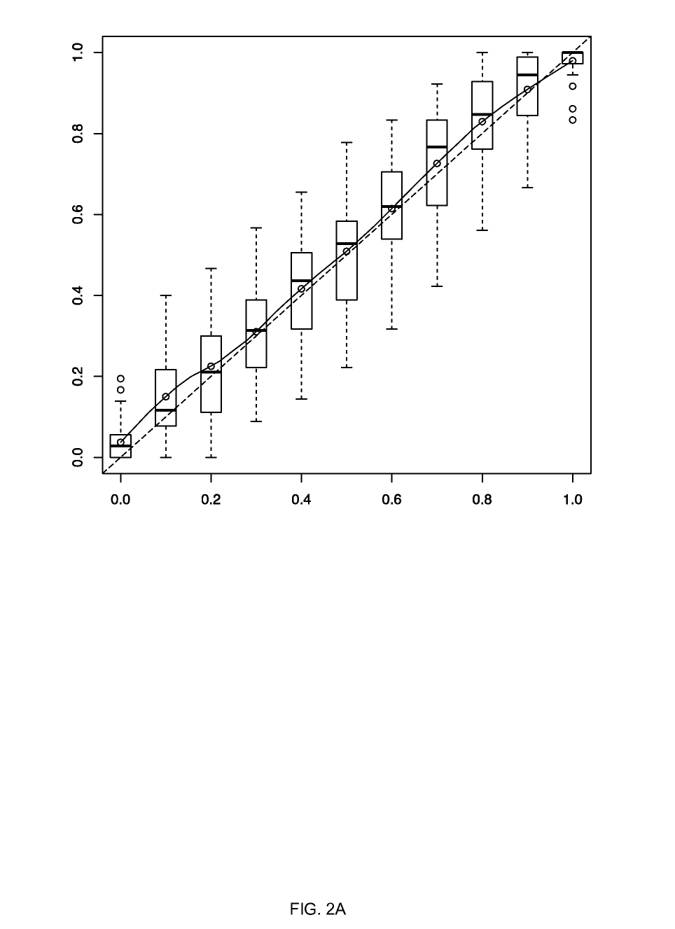

Using the final, normalized data, classifiers were created and evaluated for their ability to discriminate the clean patient samples from the polyp and/or adenoma samples. In each of fifty 70/30, training/test splits of the sample data, an elastic-net approach was used for feature selection, reducing the number of considered NMCs from more than 100,000 to approximately 200-250. These selected NMCs were then used to build SVM (sigmoid-kernel)-based classifiers. Within each iteration of the fifty training/test splits, the classifier's performance was determined on the test data as measured by AUC on ROC plots (a combined measure of sensitivity and specificity). The average AUC that resulted, 0.79+/−0.08, is shown in FIG. 1A. This AUC is significantly different from 0.5, the value that a random assay with no discriminatory power would achieve, according to the dashed line bisecting the figure. Thus, FIG. 1A provides a comparison of the testing set performance. The X-axis represents the false positive rate. The Y-axis represents the true positive rate.

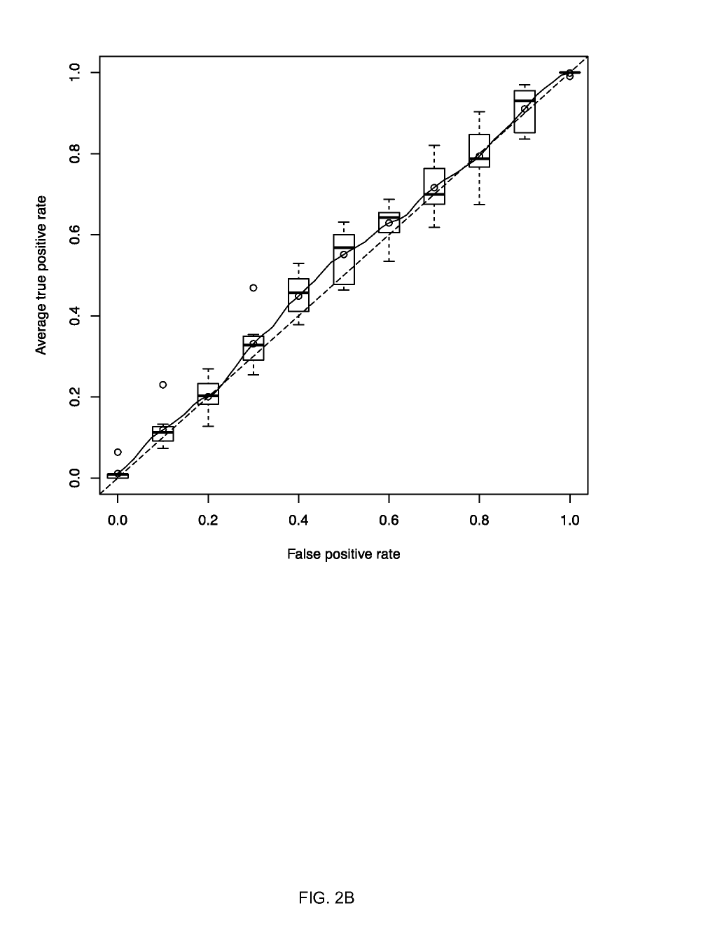

In order to confirm the robustness of the elastic-net/SVM classifier performance, the class assignments, polyp/adenoma vs. clean, were randomly permuted and the entire feature selection and classifier assembly process was performed again across fifty iterations. The resulting average AUC, 0.52+/−0.09, is shown in FIG. 2A and demonstrates that a result such as determined for the correct assignments was not likely to have arisen by chance. Thus, FIG. 2A provides a validation of the testing set performance. The X-axis represents the false positive rate. The Y-axis represents the true positive rate.

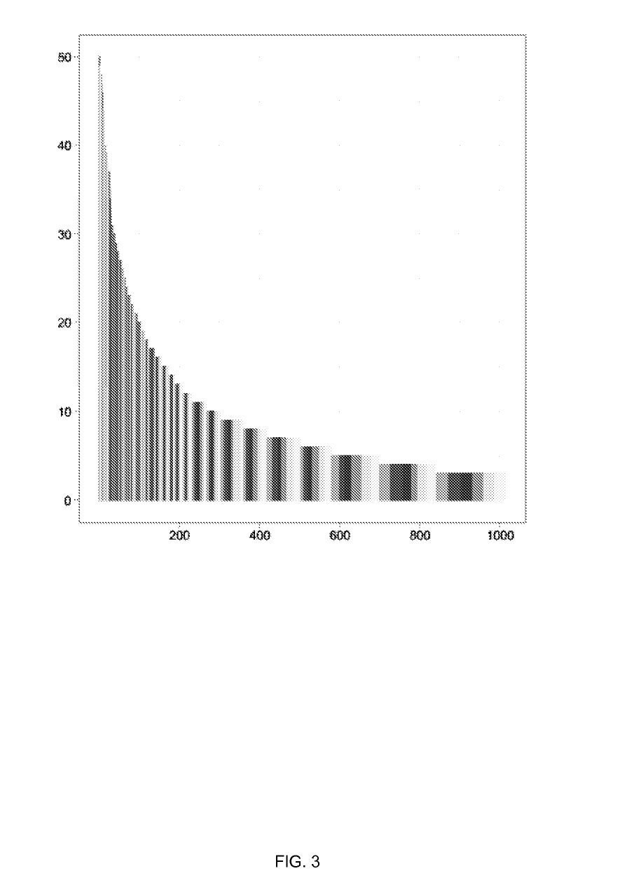

Another measure of the significance of the result is the tabulation of the frequency with which individual NMCs occur in the fifty 70/30 training/test split classifiers. In each iteration approx. 200-250 features are selected for a classifier; a feature's presence in at least 3 or more of the fifty iterations is a result not expected by chance. A pareto plot (ranked histogram) of the feature-frequency table is shown in FIG. 3. The data indicate that a large number of features are selected multiple times, suggesting robustness in their participation in discriminatory classifiers. When the most frequent features (ie., top 30 from distinct correlation groups) are selected and used to build classifiers within a nested 70(70/30)/30 analytical structure, the resulting average AUC is still significantly different than random. That result indicates that there are multiple classifiers which can be constructed from the selected feature set.

Subsets of Classifier Molecular Features

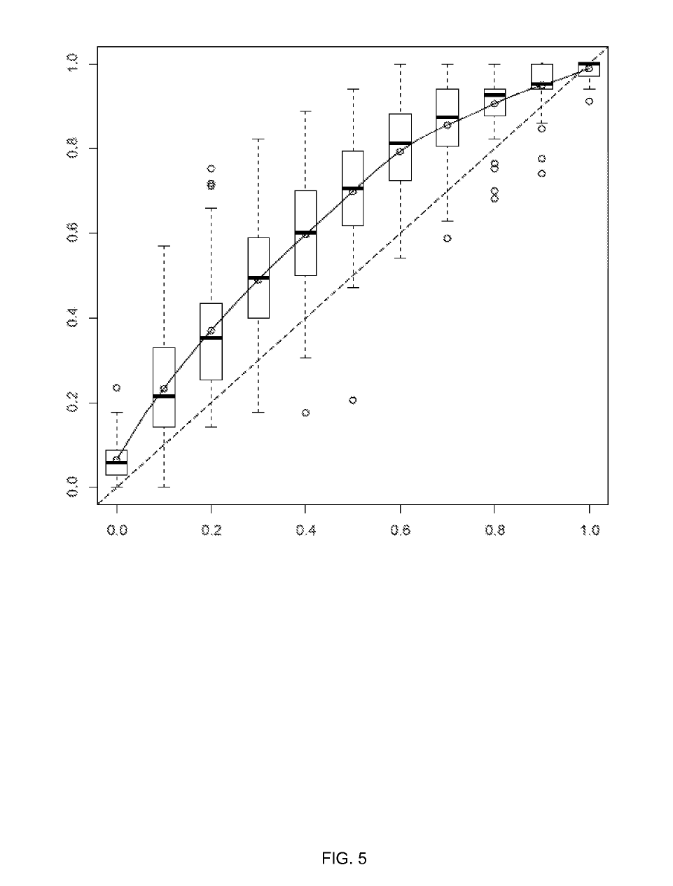

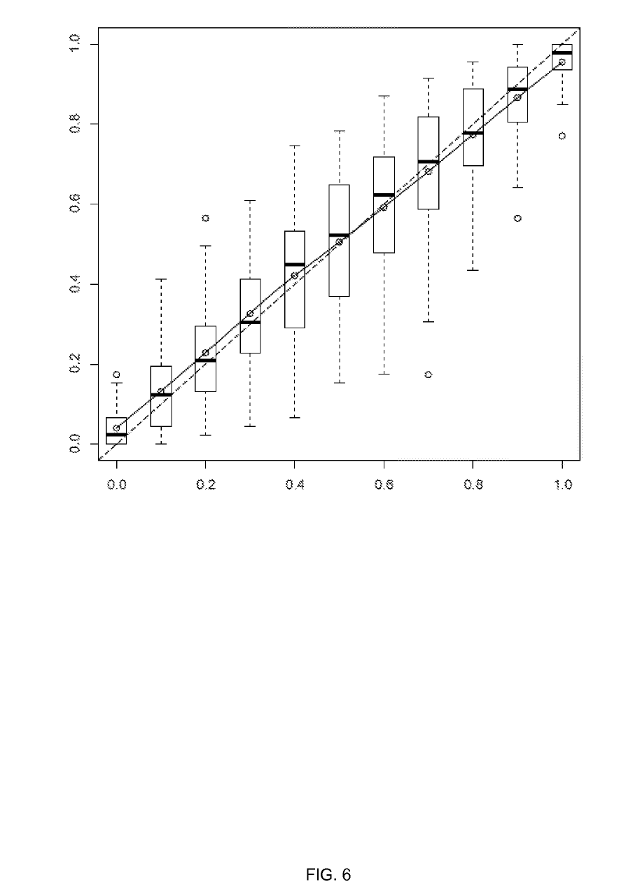

Smaller subsets of classifier features were identified by an outer loop/inner loop strategy. In this approach, the samples were divided into 50 outer loop 70/30 splits and 500 inner loop 70/30 splits. The multiple inner loops were performed for feature selection in that the SVM-classifier inner-test ROC AUC was calculated and the best 5% out of the 500 iterations were selected and the comprising features were retained. An Elastic Net was used to select a final group of features to build the outer loop SVM-classifier. For different sized classifiers, the frequency ranks for features from the selected inner loops were used to prioritize features (e.g., most frequent 10, 20, 30, etc.). The resulting classifier was evaluated on the outer loop test set and the performance AUC was measured. FIG. 5 shows the average ROC for the 50 outer loop iterations and demonstrates that a classifier of size 30 retained significant predictive value (AUC=0.645+/−0.092). In FIG. 5, the Y-axis shows the true positive rate, and the X-axis shows the false positive rate. As a confirmation that this result could not have been obtained by chance, the procedure was performed on 50 different sample sets in which the sample class assignments had been randomly re-assigned. The resulting AUC, 0.502+/−0.101, as shown in FIG. 6, was random thus confirming the robustness of the correct class assignment result. In FIG. 6, the Y-axis shows the true positive rate, and the X-axis shows the false positive rate. TABLE E7 shows that similar evidence of significant performance has been demonstrated with classifiers of size 10 features or NMCs.

TABLE E7

| Size | AUC | sd |
|---|---|---|
| 100 | 0.70 | 0.08 |
| 50 | 0.66 | 0.09 |
| 40 | 0.65 | 0.09 |
| 30 | 0.64 | 0.09 |
| 20 | 0.63 | 0.09 |
| 10 | 0.60 | 0.09 |

Identification of the Classifier Molecular Features

Mass determination of molecular features by mass spectrometry is sufficiently accurate and precise to provide unique identification. The masses of the 1014 features represented in the classifiers assembled in this Example, each present 3 or more times, are enumerated in the appended table as FIG. 7. The accurate mass is inherently uniquely identifying for a molecular feature, thus it is possible to determine the primary amino acid sequence and any post-translational modifications of these features in order to convert their measurement to an alternate presentation.

### Example 3B

Study design corresponded to the study design of Example 3A with the following additional details.

LCMS Data Acquisition and Protein Molecular Feature Quantification

Re-suspended peptides from several fractions of each patient's plasma sample were injected via UHPLC into a tandem mass spectrometer (Q-TOF) for quantitative analysis. The collected data (retention time, mass/charge ratio, and ion abundance) were analyzed to detect observed peaks referred to as molecular features. A three-dimensional peak integration algorithm determined the relative abundance of the molecular features. On average, approximately 364,000 molecular features were detected and quantified from each plasma sample.

Molecular feature data from multiple patient samples were compared after dataset overlay and alignment using a cubic spline algorithm. Only the features determined to be present in 50% or more of at least one of the patient classes (clean or polyp/adenoma) were considered for further analysis. In the case of missing patient-feature data in this set, feature values were imputed by integrating the raw ion abundance data in the a priori location of the peak as observed in other samples. Approximately 149,000 molecular features from each of the 152 patient samples comprised the final data set for subsequent classifier analysis.

Data Normalization, Feature Selection and Classifier Assembly

The quantitative data for distinct molecular features derived from a single original neutral mass were combined and summarized. For example, +2 m/z and +3 m/z features from the same parent molecule were combined by summing to a single neutral mass cluster (NMC) value. The total number of NMCs was approximately 105,000.

Details are as in Example 3A. Additionally, features were filtered by parameters used to indicate higher identification probability; For example, only features with charge state greater than 1 (z>1) were considered. This reduced the total number of NMCs used for classifier analysis to approximately 47,000.

Further to the analysis of Example 3A, in this analysis, ten rounds of 10-fold cross-validation were used to select features and build classifiers. In each, 90% of the data were used to select features using an Elastic Net algorithm with regression, the top 20 features were selected based on a ranking of the determined coefficients for the features, and then an SVM classifier with a linear kernel was constructed. This final classifier was then evaluated upon the 10% of samples held out in the test set of the given fold. Therefore, in each round of 10-fold cross validation, every sample is in the test set one and only one time. The predicted test set values from the classifier for each of the samples were used to construct a ROC plot for that round with one point for every sample. The ten ROC plots, one from each round, are averaged and plotted. For the 108 complete samples used in the analysis, and using the original colonoscopy determined diagnosis as the comparator, the median AUC for the 20 feature classifiers was 0.91. The mean AUC was 0.91±0.021. FIG. 1B.

In order to confirm the robustness of the classifier performance, the class assignments, polyp/adenoma vs. clean, were randomly permuted and the entire feature selection and classifier assembly process was performed again across ten rounds of 10-fold cross-validation as described herein. The median AUC of 0.52 and the mean AUC of 0.52±0.033 (FIG. 2B) demonstrated that a result such as determined for the correct assignments, AUC 0.91, was not likely to have arisen by chance.

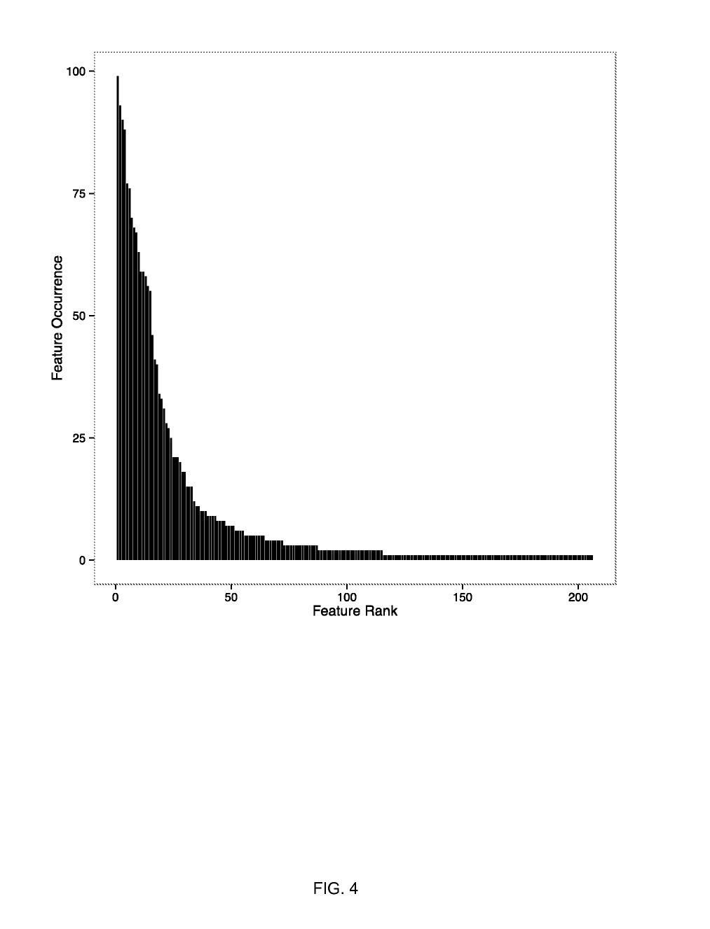

Another measure of the significance of the result is the tabulation of the frequency with which individual NMCs occur in the 100 classifiers created in the ten rounds of 10-fold cross-validation. In each iteration twenty features were selected for a classifier; a feature's presence in multiple classifiers is indicative of the robustness of the feature selection and classifier process. Using the original diagnosis to build classifiers as seen in FIG. 1B, most features were selected more than once. The most frequently selected feature was chosen in 99 out of 100 classifiers. See FIG. 4. In contrast, using random feature selection, the most frequently selected feature was chosen only three times. In all, 206 features were present in one or more of the one hundred 20-feature classifiers.

Identification of the Classifier Molecular Features

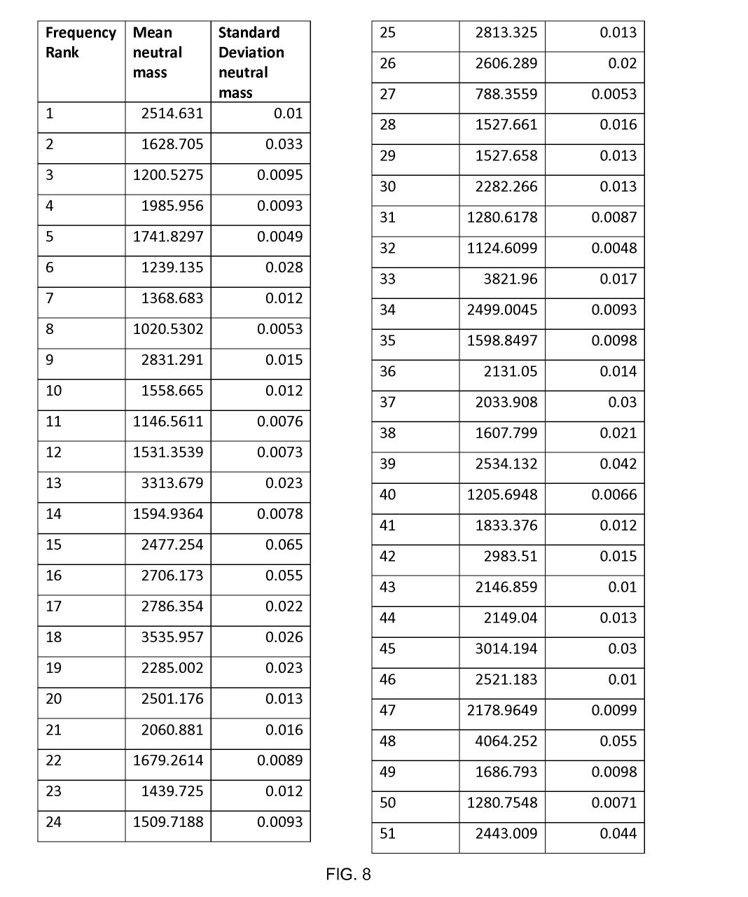

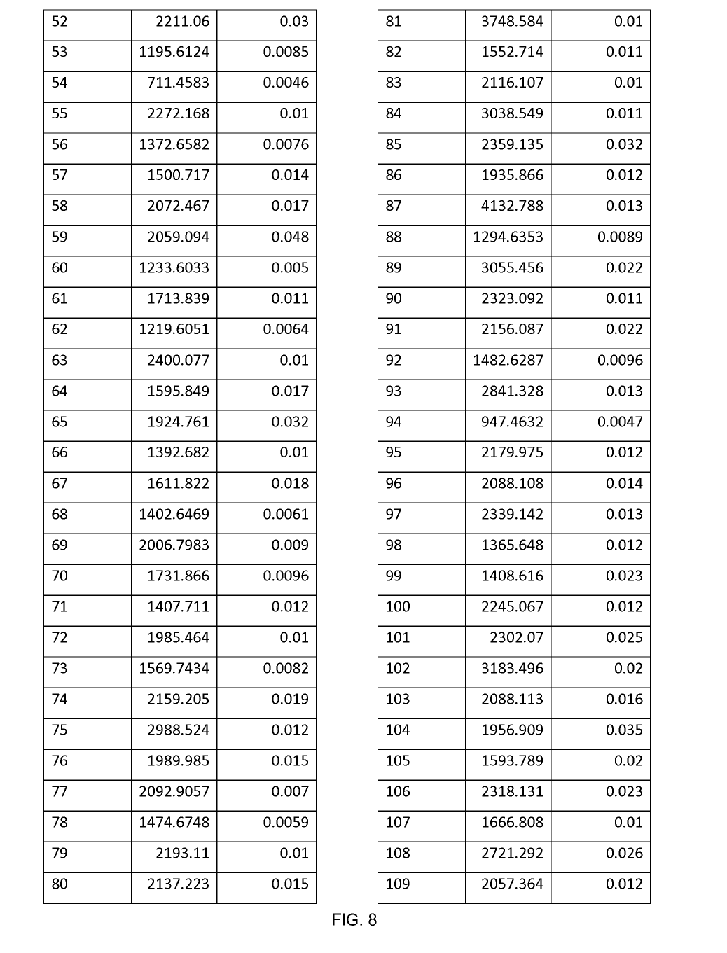

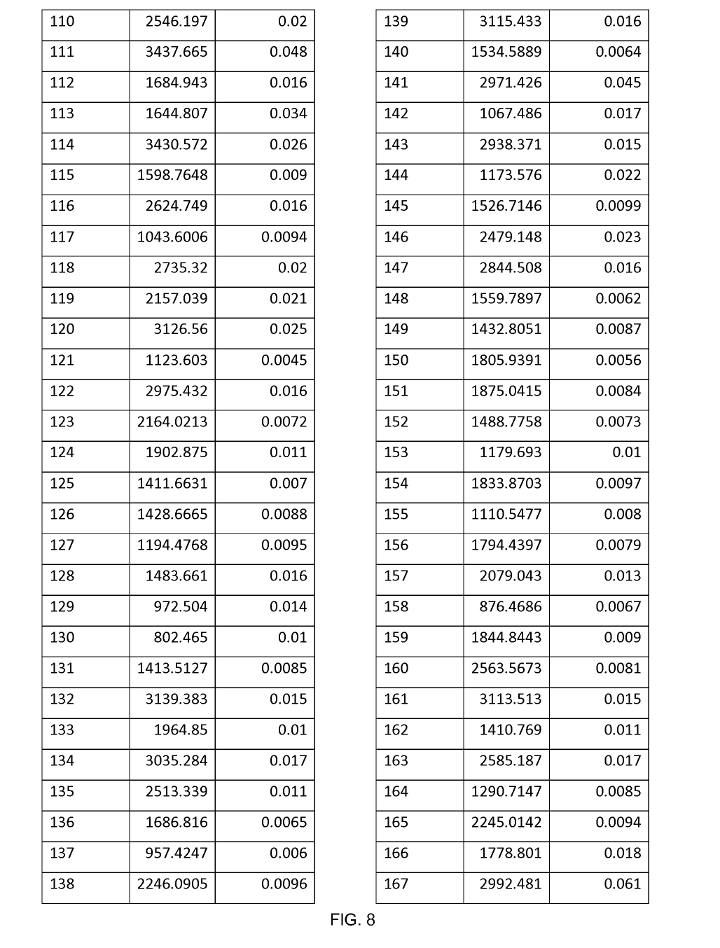

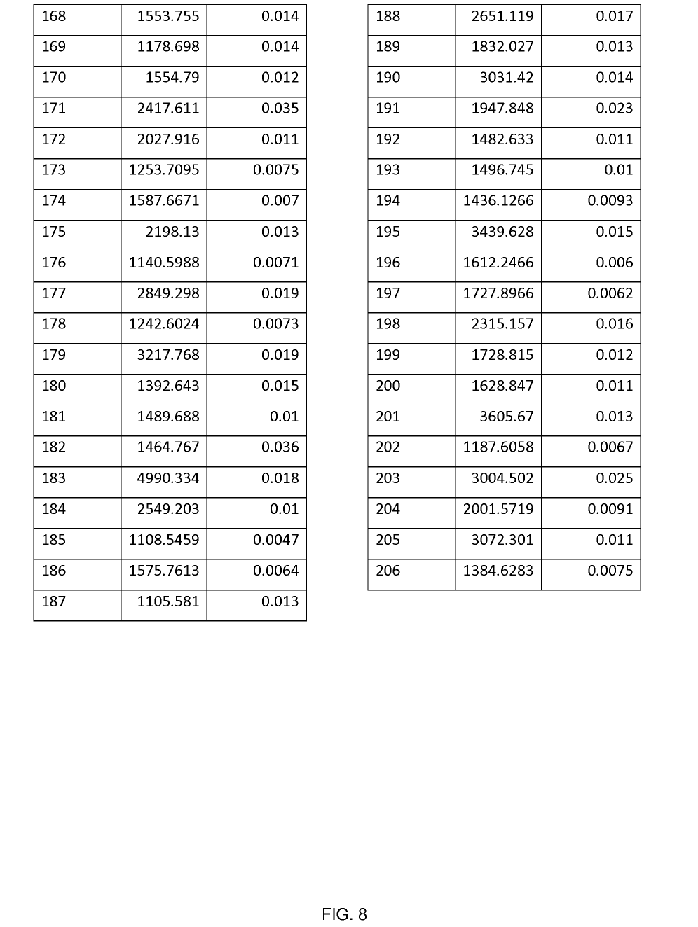

Mass determination of molecular features by mass spectrometry is sufficiently accurate and precise to provide unique identification. The masses of the 206 features represented in the classifiers assembled in this example are enumerated in the appended table as FIG. 8. The accurate mass is inherently uniquely identifying for a molecular feature, thus it is possible to determine the primary amino acid sequence and any post-translational modifications of these features in order to convert their measurement to an alternate presentation.

### Example 4

### MRM Assay Development

Initially, 188 proteins previously reported as having association to colorectal cancer were interrogated in silico to reveal potential peptide candidates for targeted proteomics profiling. From ten-of-thousands of potential tryptic peptides, a preliminary set of 1056 was selected for experimental verification. A final set of 337 peptides, representing 187 proteins, was selected from experimental verification to comprise the final multiple reaction monitoring (MRM) assay. In addition, 337 complement peptides, of exact sequence composition labeled with heavy (all carbon 13) arginine (R) or lysine (K), were incorporated as internal standards, used in the final analysis as a normalization reference.

Sample Preparation for Plasma Protein Analysis

Patient plasma protein samples were prepared for MRM LCMS measurement according to two methods, referred to as dilute and deplete.

In the dilute method, plasma samples were thawed from −80 C storage and lipids and particulates were removed by filter centrifugation. Remaining proteins were reduced to peptides by trypsin-TFE digestion, and the resulting peptides were re-suspended in acetonitrile/formic acid MRM LCMS loading buffer.

In the deplete method, plasma samples were thawed from −80 C storage and lipids and particulates were removed by filter centrifugation. The high-abundance proteins in the filtered plasma were removed by immunoaffinity column-based depletion. The lower abundance, flow-through proteins were reduced to peptides by trypsin-TFE digestion, and the resulting peptides were re-suspended in acetonitrile/formic acid MRM LCMS loading buffer.

LCMS Data Acquisition and Transition Feature Quantification

Re-suspended peptides from each patient's plasma sample were injected via UHPLC into a triple quadrupole mass spectrometer (QQQ) for quantitative analysis. The collected data (retention time, precursor mass, fragment mass, and ion abundance) were analyzed to detect observed peaks referred to as transitions.

A two-dimensional peak integration algorithm was employed to determine the area under the curve (AUC) for each of the transition peaks.

Complement peptides of exact sequence composition labeled with heavy (all carbon 13) arginine (R) or lysine (K) were utilized as internal standards for each of the 676 targeted transitions. Transition AUC values were normalized with the compliment internal standard AUC value to derive a concentration value for each transition.

Data Normalization, Feature Selection and Classifier Assembly

For the classifier assembly and performance evaluation, feature concentration values were used based upon the ratio of the raw peptide peak area to the associated labeled standard peptide raw peak area. No normalization of the underlying raw peak areas was applied. Missing values for the transitions were set to 0.

Classifier models and the associated classification performance was assessed using a 10 by 10-fold cross validation process. In this process feature selection was first applied to reduce the number of features used, followed by development of classifier model and subsequent classification performance evaluation. For each of the 10-fold cross validations, the data were segregated into 10 splits each containing 90% of the samples as a training set, and the remaining 10% of the samples as a testing set. In this process each of the 95 total samples was evaluated one time in a test set. The feature selection and model assembly process was performed using the training set only, and these models were then applied to the testing set to evaluate classifier performance.

To further assess the generalization of the classification performance, this entire 10-fold cross validation procedure was repeated 10 times, each with a different sampling of training and testing sets.

The total number of transition features used for classifier analysis was 674. To explore the classification performance with few numbers of features, Elastic Network feature selection was applied prior to building the classification model. In this process, Elastic Network models were built and the model giving 20 transition features was used in the development of the classification model. Because each fold of the cross-fold validation process has its own feature selection step, different features may be selected with each fold, so the total number of features used in the models across the by 10-fold cross validation process will be greater-than-or-equal to 20.

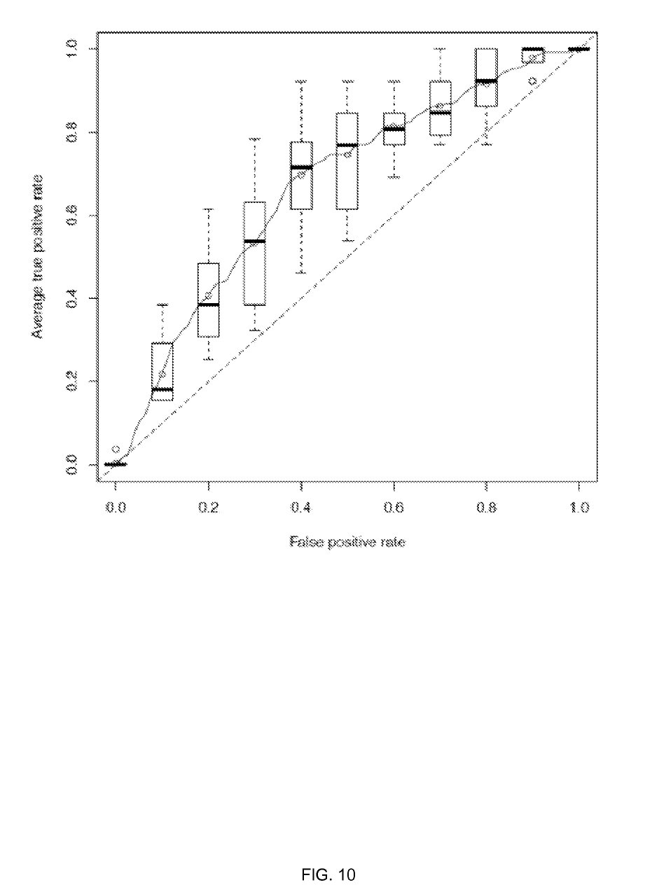

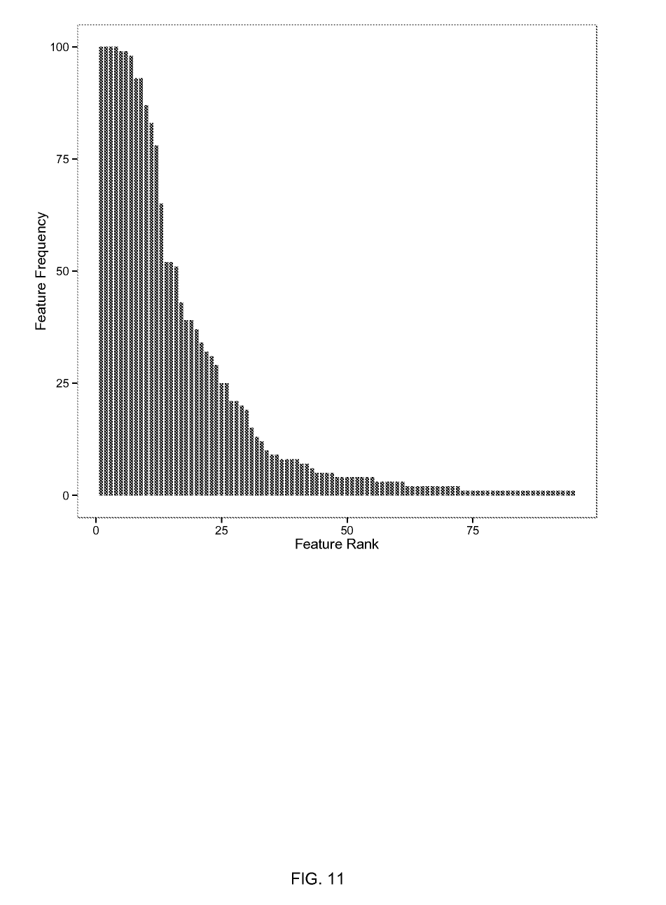

After the feature selection step, a classifier model was built using the support vector machine (SVM) algorithm with a linear kernel. After construction of the classifier model on the training set, it was directly applied without modification to the testing set and the associated receiver operator characteristic (ROC) curve was generated from which the area under the curve (AUC) was computed. In the 10 by 10-fold cross validation process, a mean test set AUC of 0.76+/−0.035 was obtained FIG. 10 indicating the ability for the classification model to discriminate colorectal cancer and normal patient samples. To further assess the features selected during the feature selection process, a frequency/rank plot was produced FIG. 11. This plot shows several features that were selected in all or almost all of the cross validation fold, highlighting their utility in distinguishing colorectal cancer from normal samples. The list of features identified through the classification process are listed in FIG. 12.

### Study Design and Patient Sample Collection

| | Control | CRC Disease |
|---|---|---|
| Female | 24 | 23 |
| Male | 24 | 24 |
| | | p = 1 |
| Age | 65.0 +/− 9.7 | 65.5 +/− 9.6 |
| (mean +/− stdev in years) | | |
| | | p = 0.82 |

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1.-10. (canceled)

11. A method of detecting the presence or absence of an adenoma or polyp of the colon in a subject, wherein said subject has no symptoms or family history of adenoma or polyps of the colon, said method comprising the steps of:
(a) obtaining a biological sample from said subject;
(b) performing an analysis of the biological sample for the presence and amount of one or more proteins and/or peptides;
(c) comparing the presence and amount of one or more proteins and/or peptides from said biological sample to a control reference value; and
(d) correlating the presence and amount of one or more proteins and/or peptides with the subject's adenoma or polyp status;
wherein said analysis detects the presence and/or amount of one or more neutral mass clusters from the first 10 neutral mass clusters of FIG. 8, and wherein said neutral mass cluster has a classifier frequency when tested according to a 70/30 training/test for split classifiers, wherein said classifier frequency is selected from at least 3 out of 50, at least 10 out of 50, at least 20 out of 50, at least 30 out of 50, and at least 40 out of 50.

12.-139. (canceled)
